(19) United States
(12) Patent Application Publication
(10) Pub. No.: US 2025/0188498 A1
(43) Pub. Date: Jun. 12, 2025

(54) GENETICALLY MODIFIED PSEUDOMONAS HOST CELLS AND METHODS USEFUL FOR PRODUCING ISOPRENOL

(71) Applicants: THE REGENTS OF THE UNIVERSITY OF CALIFORNIA, Oakland, CA (US); BATTELLE MEMORIAL INSTITUTE, Richland, WA (US)

(72) Inventors: Taek Soon LEE, Berkeley, CA (US); Xi WANG, Emeryville, CA (US); Ian YUNUS, Berkeley, CA (US); Joonhoon KIM, Richland, WA (US); Deepanwita BANERJEE, Emeryville, CA (US); Aindrila MUKHOPADHYAY, San Francisco, CA (US)

(21) Appl. No.: 18/976,085

(22) Filed: Dec. 10, 2024

Related U.S. Application Data
(60) Provisional application No. 63/608,355, filed on Dec. 11, 2023.

(51) Int. Cl. C12P 7/04; C12N 1/20; C12N 9/00; C12N 9/04; C12N 9/06; C12N 9/10; C12N 9/12; C12N 9/16; C12N 9/88

(52) U.S. Cl. CPC C12P 7/04; C12N 1/20; C12N 9/0006; C12N 9/0016; C12N 9/1029; C12N 9/1205; C12N 9/16; C12N 9/88; C12N 9/93; C12Y 101/0103; C12Y 101/01041; C12Y 104/01013; C12Y 203/01009; C12Y 203/0301; C12Y 207/01036; C12Y 207/09002; C12Y 301/03001; C12Y 301/03002; C12Y 401/01033; C12Y 401/03001; C12Y 401/03004; C12Y 604/01001

(57) ABSTRACT

The present invention provides for a method to increase production of isoprenol by a genetically modified Pseudomonas cell, the method comprising: (a) providing a genetically modified Pseudomonas cell comprising one or more of heterologous genes encoding: MvaE, AtoB, MvaS, MK, PMD_{HKQ}, AphA, and PhoA; and (b) culturing or growing the genetically modified Pseudomonas cell in a medium to produce isoprenol; wherein (i) the genetically modified Pseudomonas cell is deleted, knocked out, or reduced in expression of one or more of the following endogenous genes: a gene at PP_2675 locus (or a deletion of the PP_2675 locus), phaABC, mvaB, hbdH, ldhA, gntZ, ppsA, pycAB, gltA, and aceA, and/or (ii) the medium comprises one or more amino acids that reduce the catabolism of isoprenol.

### CROSS REFERENCE TO RELATED APPLICATIONS

This application claims priority to U.S. Provisional Patent Application Ser. No. 63/608,355, filed Dec. 11, 2023, which are hereby incorporated by reference.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of producing isoprenol.

### REFERENCE TO A “SEQUENCE LISTING” SUBMITTED AS ASCII TEXT FILES VIA EFS-WEB

Reserved.

### BACKGROUND OF THE INVENTION

Increasing concerns of climate change and energy security have necessitated microbial biosynthesis to produce biofuels from renewable carbon source as a sustainable and stable alternative to the fossil fuel-based approaches [1,2]. Biofuels research to date has predominantly focused on conversion of sugars (hexoses and pentoses), the primary components of deconstructed lignocellulosic biomass [3]. However, lignin is another major component of lignocellulosic biomass and its catabolism has also been extensively studied recently [4-6]. Development of a new microbial chassis that enables full utilization of the lignocellulosic biomass-derived carbon sources is critical to achieve economically viable biofuel production [7].

Pseudomonas putida KT2440 has recently emerged as a promising microbial host due to its capability of utilizing a broad range of carbon sources and its high tolerance to xenobiotics [8]. As P. putida is usually isolated from soils [9], the natural living environment conveys to P. putida versatile metabolism to degrade different types of substrates as carbon sources and it is adapted to tolerate various physicochemical stresses. Particularly, P. putida can utilize lignin-derived intermediates and aromatics, such as p-coumarate, benzoate, toluene as sole carbon sources, and thus has great potential to be developed as a new microbial workhorse to convert renewable carbon sources during bio-based production. P. putida KT2440 has been generally recognized as safe (GRAS) and is widely used for metabolic engineering studies as its full genome sequence is available [10]. It can share some genetic parts (plasmid backbone, promoter, RBS, etc.) with Escherichia coli, which could facilitate the genetic modification in P. putida. However, P. putida also showed different sugar metabolism from the model hosts that use classic glycolysis pathway, such as E. coli, Saccharomyces cerevisiae. P. putida oxidizes glucose to gluconate and 2-ketogluconate in the periplasm, followed by the phosphorylation to 6-phophogluconate (6PG) toward Entner-Doudoroff (ED) pathway (FIG. 1). Due to the lack of phosphofructokinase (PFK) that catalyzes the rate-limiting phosphorylation of fructose-6-phosphate (F6P) to fructose-1,6-diphosphate (FBP) in glycolysis, P. putida doesn't catabolize glucose through the typical glycolysis but by the ED pathway [11].

Microbial production of isoprenoids has been considered a critical route for developing biofuels [12]. The biosynthesis of isoprenoids starts with two key isoprene units, isopentenyl diphosphate (IPP) and dimethylallyl diphosphate (DMAPP), naturally synthesized by two isoprenoid pathways: the mevalonate (MVA) pathway and non-mevalonate (methylerythritol 4-phosphate, MEP) pathway [13], respectively. IPP and DMAPP are precursors of isopentenols (C₅) [14], and they can also be condensed to geranyl diphosphate (GPP) and farnesyl diphosphate (FPP) to produce monoterpenes (C₁₀) [15,16] and sesquiterpenes (C₁₅) [17,18], respectively. All C₅, C₁₀, and C₁₅ isoprenoids are important candidates for fuel, especially jet fuel replacements [19]. Typical isoprenoid fuel molecules include isoprenol (C₅) [20], limonene (C₁₀) [15], 1,8-cincole (C₁₀) [16], bisabolene (C₁₅) [17], epi-isozizaene (C₁₅) [18], etc. Among them, isoprenol (3-methyl-3-buten-1-ol) has received more attention due to its increasing applications as a valuable drop-in fuel molecule and important precursor of commodity chemicals. For example, US Navy has recently developed a high-performance jet fuel, 1,4-dimethylcyclooctane (DMCO), which can be produced from isoprenol [21]. Isoprenol is the dephosphorylated molecule of isopentenyl phosphate (IP) [22]. Unlike monoterpenes and sesquiterpenes, isoprenol synthesis does not require IPP which is toxic to cell growth [23]. An IPP-bypass pathway was developed to overcome the IPP toxicity and showed advantages in isoprenol (C₅) production in both E. coli and yeast [20].

Given that P. putida is emerged as a new workhorse strain, it has attracted interests for engineering of isoprenoid production [25-27]. For example, there have been a few literatures for isoprenoids production in P. putida, and mostly, the endogenous MEP pathway was engineered for the isoprenoids production and frequently focused on the oxidation of terpenes using P450 enzymes as P. putida is known to be tolerant to oxidative stress [25]. The heterologous MVA pathway was also expressed in P. putida, but the performance was not as good as what was shown in E. coli when a similar engineering strategy was attempted, and only a low productivity and titers were achieved [26].

### SUMMARY OF THE INVENTION

The present invention provides for a method to increase production of isoprenol by a genetically modified Pseudomonas cell, the method comprising: (a) providing a genetically modified Pseudomonas cell comprising one or more, or all, of heterologous genes encoding: MvaE, AtoB, MvaS, MK, PMD_{HKQ}, AphA, and PhoA; and (b) culturing or growing the genetically modified Pseudomonas cell in a medium to produce isoprenol; wherein (i) the genetically modified Pseudomonas cell is deleted, knocked out, or reduced in expression of one or more of the following endogenous genes: a gene at PP_2675 locus (or a deletion of the PP_2675 locus), phaABC, mvaB, hbdH, IdhA, gntZ, ppsA, pycAB, gltA, and aceA, and/or (ii) the medium comprises one or more amino acids that reduce the catabolism of isoprenol.

The present invention provides for a method used for production of isoprenol in a novel host, Pseudomonas putida KT2440. Pseudomonas putida KT2440 is known to catabolize or consume isoprenol.

In some embodiments, the genetically modified Pseudomonas cell is deleted, knocked out, or reduced in expression of one or more of the following endogenous genes: a gene at PP_2675 locus (or a deletion of the PP_2675 locus), phaABC, mvaB, hbdH, IdhA, gntZ, ppsA, pycAB, gltA, and aceA. In some embodiments, the genetically modified Pseudomonas cell comprises the following genotype: phaABC mvaB hbdH ldhA PP_2675. In some embodiments, the genetically modified Pseudomonas cell comprises the following genotype: ΔphaABC ΔmvaB ΔhbdH ΔldhA ΔPP_2675.

In some embodiments, the amino acid that reduces the catabolism of isoprenol is glutamate, glutamine, arginine, glycine, serine, valine leucine, and/or alanine, or a mixture thereof. In some embodiments, that the amino acid is glutamate and/or glutamine, or a mixture thereof. In some embodiments, that the amino acid is one or more described in Table 2, or a mixture thereof.

In some embodiments, the Pseudomonas cell is a Pseudomonas putida cell. In some embodiments, the Pseudomonas putida cell is a Pseudomonas putida KT2440 cell.

In some embodiments, the Pseudomonas cell is a genetically modified such that the Pseudomonas cell comprises an isopentenyl-diphosphate (IPP)-bypass isoprenol biosynthetic pathway. In some embodiments, the genetic modifications of the IPP-bypass isoprenol biosynthetic pathway are taught in PCT International Patent Application No. PCT/US2016/018984; and U.S. Pat. Nos. 10,273,506; 10,814,724; and, 11,660,961; hereby all incorporated by reference in their entireties.

In some embodiments, the genetically modified Pseudomonas cell comprises: (a) an increased expression of phosphomevalonate decarboxylase (PMD) (b) an increased expression of a phosphatase capable of converting isopentenol into 3-methyl-3-butenol, (c) the genetically modified host cell does not express, or has a decreased expression of one or more of NudB, PMK, and/or PMD, and (d) one or more further enzymes capable of converting isopentenol and/or 3-methyl-3-butenol into a third compound, such as isoprene. In some embodiments, the decreased expression is a disruption of the promoter or knock out of the gene encoding the enzyme.

In some embodiments, the genetically modified Pseudomonas cell comprises an increased expression of one or more of AtoB, hydroxymethylglutaryl-CoA synthase (HMGS), hydroxymethylglutaryl-CoA reductase (HMGR), and/or MK.

In some embodiments, one or more of the described expressed enzymes, such as PMD, phosphatase, AtoB, HMGS, HMGR, and/or MK, are encoded on one or more nucleotide sequences which are in one or more nucleic acids which are transformed into the genetically modified Pseudomonas cell, or host cell prior to genetic modification. In some embodiments, the nucleotide sequences encoding the one or more enzymes are operatively linked to one or more promoters capable of transcription in the genetically modified Pseudomonas cell. In some embodiments, each nucleic acid of the one or more nucleic acids is a vector capable of stable introduction into and/or maintenance in the Pseudomonas cell.

In some embodiments, the method further comprises: culturing or growing the genetically modified Pseudomonas cell under a condition wherein PMD and/or phosphatase are expressed, and/or recovering the isoprenol, isopentenol, and/or 3-methyl-3-butenol.

In some embodiments, the culturing or growing step further comprises expressing AtoB, HMGS, HMGR, and/or MK. In some embodiments, the (b) culturing step is under an anaerobic or microaerobic condition.

In some embodiments, one or more of the enzymes, including PMD, phosphatase, AtoB, HMGS, HMGR, and MK, is an engineered enzyme, or homologous, mutant or variant enzymes having the same enzymatic activity, with an amino acid sequence having equal to or more than 70%, 80%, 90%, 95%, or 99% identity to the amino acid sequence of the corresponding wild-type enzyme, such as the specific enzymes described in PCT International Patent Application No. PCT/US2016/018984; and U.S. Pat. Nos. 10,273,506; 10,814,724; and, 11,660,961.

In some embodiments, the MK comprises a polypeptide having MK enzymatic activity, and an amino acid sequence having at least equal to or more than 70%, 80%, 90%, 95%, or 99% identity to the amino acid sequence of the corresponding wild-type Saccharomyces cerevisiae or Methanosarcina mazei MK, and one or more conserved amino acid residues in MK. In some embodiments, the MK is a wild-type MK of any species, such as any yeast or bacteria species.

In some embodiments, the PMD comprises a polypeptide having PMD enzymatic activity, and an amino acid sequence having at least equal to or more than 70%, 80%, 90%, 95%, or 99% identity to the amino acid sequence of the corresponding wild-type Saccharomyces cerevisiae PMD, and one or more conserved amino acid residues in PMD. In some embodiments, the PMD is a wild-type PMD of any species, such as any yeast or bacteria species. In some embodiments, the PMD comprises R74H, R147K, and M212Q corresponding to the Saccharomyces cerevisiae PMD.

In some embodiments, the PMK comprises a polypeptide having PMK enzymatic activity, and an amino acid sequence having at least equal to or more than 70%, 80%, 90%, 95%, or 99% identity to the amino acid sequence of the corresponding wild-type Saccharomyces cerevisiae PMK, and one or more conserved amino acid residues in PMK. In some embodiments, the PMK is a wild-type PMK of any species, such as any yeast or bacteria species.

In some embodiments, the AtoB comprises a polypeptide having AtoB enzymatic activity, and an amino acid sequence having at least equal to or more than 70%, 80%, 90%, 95%, or 99% identity to the amino acid sequence of the corresponding wild-type Escherichia coli AtoB, and one or more conserved amino acid residues in AtoB. In some embodiments, the AtoB is a wild-type AtoB of any species, such as any yeast or bacteria species.

In some embodiments, the PhoA comprises a polypeptide having PhoA enzymatic activity, and an amino acid sequence having at least equal to or more than 70%, 80%, 90%, 95%, or 99% identity to the amino acid sequence of the corresponding wild-type Escherichia coli PhoA, and one or more conserved amino acid residues in PhoA. In some embodiments, the PhoA is a wild-type PhoA of any species, such as any yeast or bacteria species.

In some embodiments, the AphA comprises a polypeptide having AphA enzymatic activity, and an amino acid sequence having at least equal to or more than 70%, 80%, 90%, 95%, or 99% identity to the amino acid sequence of the corresponding wild-type Escherichia coli AphA, and one or more conserved amino acid residues in AphA. In some embodiments, the AphA is a wild-type AphA of any species, such as any yeast or bacteria species.

In some embodiments, the MvaE comprises a polypeptide having MvaE enzymatic activity, and an amino acid sequence having at least equal to or more than 70%, 80%, 90%, 95%, or 99% identity to the amino acid sequence of the corresponding wild-type Enterococcus faecalis MvaE, and one or more conserved amino acid residues in MvaE. In some embodiments, the MvaE is a wild-type MvaE of any species, such as any yeast or bacteria species.

In some embodiments, the MvaS comprises a polypeptide having MvaS enzymatic activity, and an amino acid sequence having at least equal to or more than 70%, 80%, 90%, 95%, or 99% identity to the amino acid sequence of the corresponding wild-type Enterococcus faecalis MvaS, and one or more conserved amino acid residues in MvaS. In some embodiments, the MvaS is a wild-type MvaS of any species, such as any yeast or bacteria species.

In some embodiments, the HMGS comprises a polypeptide having HMGS enzymatic activity, and an amino acid sequence having at least equal to or more than 70%, 80%, 90%, 95%, or 99% identity to the amino acid sequence of the corresponding wild-type Staphylococcus aureus, Saccharomyces cerevisiae, or Enterococcus faecalis HMGS, and one or more conserved amino acid residues in HMGS. In some embodiments, the HMGS is a wild-type HMGS of any species, such as any yeast or bacteria species.

In some embodiments, the HMGR comprises a polypeptide having HMGR enzymatic activity, and an amino acid sequence having at least equal to or more than 70%, 80%, 90%, 95%, or 99% identity to the amino acid sequence of the corresponding wild-type Staphylococcus aureus, Saccharomyces cerevisiae, or Enterococcus faecalis HMGR, and one or more conserved amino acid residues in HMGR. In some embodiments, the HMGR is a wild-type HMGR of any species, such as any yeast or bacteria species.

In some embodiments, any of the enzymes comprises a polypeptide having the wild-type enzyme's enzymatic activity, and an amino acid sequence having at least equal to or more than 70%, 80%, 90%, 95%, or 99% identity to the amino acid sequence of a corresponding wild-type enzyme, and one or more conserved amino acid residues in the enzyme. In some embodiments, the enzyme is a wild-type enzyme of any species, such as any yeast or bacteria species.

In some embodiments, one or more of the enzymes, including PMD, phosphatase, AtoB, HMGS, HMGR, and MK, is heterologous to the host cell.

In some embodiments, the method results in the genetically modified Pseudomonas cell producing more isoprenol than a unmodified wild-type Pseudomonas cell. In some embodiments, the method results in the genetically modified Pseudomonas cell producing equal to or more than about 200 mg/L, 250 mg/L, 300 mg/L, 350 mg/L, 400 mg/L, 450 mg/L, 500 mg/L, 550 mg/L, 600 mg/L, 650 mg/L, 700 mg/L, 750 mg/L, 760 mg/L, or 770 mg/L of isoprenol, or the genetically modified Pseudomonas cell produces isoprenol within a range of any two of the preceding titers.

We identify two methods to minimize isoprenol consumption in Pseudomonas putida KT2440: first by specific amino acids (such as glutamate) supplementation and second, by gene deletion of PP_2675 locus. We also demonstrate the isoprenol production in P. putida KT2440 for the first time by introducing heterologous pathways including isopentenyl-diphosphate (IPP)-bypass isoprenol biosynthetic pathway. The heterologous pathways including isopentenyl-diphosphate (IPP)-bypass isoprenol biosynthetic pathway, among other features and elements are taught in PCT International Patent Application No. PCT/US2016/018984; and U.S. Pat. Nos. 10,273,506; 10,814,724; and, 11,660,961; hereby all incorporated by reference in their entireties. We significantly improve the isoprenol titer by optimizing the pathway protein expression levels. Our invention also describes the identification of knockout genes (such as phaABC, mvaB, hbdH, ldhA, and PP_2675) that substantially improves the isoprenol production titer. These gene knockout targets were suggested by combining two computational methods described herein.

The present invention provides for a Pseudomonas putida KT2440 strain with reduced level of isoprenol catabolism and is capable of producing isoprenol. In order to increase the technology readiness level, we will need to further improve the isoprenol titer and also ideally integrate the isoprenol pathway to the chromosome. Pseudomonas putida KT2440 is a versatile host that can readily utilize aromatic compounds and be engineered to consume various sugars generated from lignocellulosic biomass.

In some embodiments, the Pseudomonas cell is genetically modified to be capable of metabolizing one or more compounds that the unmodified Pseudomonas cell is incapable of metabolizing in nature. In some embodiments, the one or more compounds is an aromatic compound obtained from lignocellulosic hydrolysate. Examples of such aromatic compounds are ferulic acid, 4-Hydroxybenzoic acid, protocatechuic acid, vanillic acid, salicylic acid, syringic acid, p-Coumaric acid, vanillin, catechol, syringaldehyde, and phenol. Examples of suitable genetic modifications to enable the Pseudomonas cell to metabolize such aromatic compounds are taught in the following: overexpression of eruloyl-CoA synthetase (Fcs), enoyl-CoA hydratase/aldolase (Ech), vanillin dehydrogenase (Vdh), p-hydroxybenzoate hydroxylase (PobA), and vanillic acid O-demethylase oxygenase (VanAB).

In some embodiments, the method further comprises: extracting or separating the isoprenol from the Pseudomonas cells, and/or culture or medium, to form an isolated or purified isoprenol.

In some embodiments, the gene of each enzyme is operatively linked to a promoter capable of expressing each enzyme in the Pseudomonas cell. In some embodiments, the culturing or growing step (b) comprises the Pseudomonas cell growing by respiratory cell growth. In some embodiments, the culturing or growing step (b) takes place in a batch process or a fed-batch process, such as a high-gravity fed-batch process.

In some embodiments, the culture comprises a biomass, such as a lignocellulosic biomass, or hydrolysate thereof. In some embodiments, the biomass is obtained from softwood feedstock (such as poplar), hardwood feedstock, grass feedstock, and/or agricultural feedstock, or mixture thereof.

In some embodiments, the culture or medium comprises a rich medium, such as LB (Lysogeny-Broth) or comprising one or more ingredients of LB, such as tryptone and/or yeast extract. In some embodiments, the culture or medium comprises hydrolysates derived or obtained from a biomass, such as a lignocellulosic biomass. In some embodiments, the culture or medium comprises one or more aromatic compound(s), such as aromatic compounds obtained from a lignocellulosic hydrolysate. In some embodiments, the culture or medium comprises one or more carbon sources, such as a sugar, such as glucose, xylose, or galactose, or glycerol, or a mixture thereof. In some embodiments, the carbon source is fermentable. In some embodiments, the carbon source is non-fermentable. In some embodiments, the culture or medium comprises urea as a nitrogen source. In some embodiments, the culture or medium comprises an amino acid, such as serine and/or glycine. In some embodiments, the culture or medium comprises an ionic liquid (IL).

In some embodiments, the invention comprises the use of a heterologous codon-optimized version of the nucleic acid encoding the enzyme(s) described herein which are optimized to the Pseudomonas cell.

The present invention provides for a genetically modified Pseudomonas cell comprising (a) one or more, or all, of heterologous genes encoding: MvaE, AtoB, MvaS, MK, PMD_{HKQ}, AphA, and PhoA; and (b) a deletion, knock out, or reduced in expression of one or more of the following endogenous genes: a gene at PP_2675 locus, or a deletion of the PP_2675 locus, phaABC, mvaB, hbdH, IdhA, gntZ, ppsA, pycAB, gltA, and aceA.

In some embodiments, the genetically modified Pseudomonas cell comprises one of more proteins and/or enzymes described herein, and/or one or more nucleic acid encoding the one or more proteins and/or enzymes thereof, each one or more nucleic acid operatively linked to a promoter capable of expressing the one or more proteins and/or enzymes in the Pseudomonas cell. In some embodiments, genetically modified Pseudomonas cell comprises one of more plasmids described herein. In some embodiments, the genetically modified Pseudomonas cell is capable of producing epi-isozizaene (C₁₅), and/or any other compound described herein.

The present invention provides for a medium or culture comprising (a) a genetically modified Pseudomonas cell of claim 1, and (b) one or more amino acids that reduce the catabolism of isoprenol.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 Central metabolism and isoprenol production pathways in P. putida. Isoprenol production pathways, including the MEP pathway, the original MVA pathway, and the IPP-bypass MVA pathway are presented and the key engineering efforts to overexpress or knockout genes are presented in red. G6P, glucose-6-phosphate; F6P, fructose-6-phosphate; FBP, fructose-1,6-diphosphate; G3P, glyceraldehyde-3-phosphate; 3PG, 3-phosphoglycerate; PEP, phosphoenolpyruvate; 2 KG, 2-ketoglutarate; MVA, mevalonate; MVAP, mevalonate phosphate; MVAPP, mevalonate diphosphate; IPP, isopentenyl diphosphate; DMAPP, dimethylallyl diphosphate; IP, isopentenyl phosphate; PHA, polyhydroxyalkanoate; MK, mevalonate kinase; PMK, phosphomevalonate kinase; PMD, phosphomevalonate decarboxylase; NudB, dihydroncopterin triphosphate diphosphatase. MvaES, HMGS and HMGR genes from Enterococcus faecalis; phaABC, PHA synthase.

FIG. 2 Engineering heterologous pathway for isoprenol production in P. putida. (A) Configuration of P. putida strains with the engineered isoprenol pathway plasmids. (B-D) Production results by the engineered P. putida strains from 1% glucose. (B) Cell growth. The initial OD₆₀₀ of 0 h was set at 0.05. (C) Isoprenol production. Isoprenol levels of different strains were not detectable at 0 h; (D) Glucose consumption. Error bars indicate one standard deviation of triplicates. NudB, dihydroncopterin triphosphate diphosphatase (E. coli); AtoB, acetoacetyl-CoA synthase (E. coli); HMGS, HMG-COA synthase; HMGR, HMG-COA reductase; MvaS, HMG-COA synthase; MvaE, HMG-COA reductase; MK, mevalonate kinase; PMK, phosphomevalonate kinase; PMD, phosphomevalonate decarboxylase. The footnote of enzymes indicates their sources: Sc, S. cerevisiae; Sa, Staphylococcus aureus; Ef, Enterococcus faecalis. BBR1, broad host range replication origin; KanR, kanamycin-resistant antibiotic marker.

FIG. 3 Optimizing isoprenol production. Isoprenol production from 2% glucose with different background strains, top-portion MVA pathway, and MK-PMD genes. Error bars indicate one standard deviation of triplicates. MK, mevalonate kinase; PMD, phosphomevalonate decarboxylase. Sc, S. cerevisiae; Mm, Methanosarcina mazei; HKQ, a mutant of PMDSc containing three mutations (R74H, R147K, and M212Q).

FIG. 4 Investigation of isoprenol consumption in P. putida. (A) Isoprenol consumption by P. putida ΔphaABC strain (JPUB_019964) in M9 minimal medium and EZ-rich medium containing 1% glucose or no glucose, respectively. (B) Isoprenol consumption by P. putida ΔphaABC strain in M9 minimal medium supplemented with individual amino acid. The working concentrations of 8 amino acids were shown in Table 2. Control, no amino acid supplemented; Mix, the mixture of all 8 amino acids with the same individual concentration. Error bars indicate one standard deviation of triplicates.

FIG. 5 Metabolite analysis of L-glutamate supplement during isoprenol consumption in P. putida (JPUB_019964). Metabolites under four conditions were investigated after 24 hours. Control, no isoprenol and no L-glutamate added; Isoprenol only, 1 g/L isoprenol added; L-Glu only, 6 mM L-glutamate added; Isoprenol+L-Glu, 1 g/L isoprenol and 6 mM L-glutamate added. Error bars indicate one standard deviation of triplicates.

FIG. 6 Isoprenol production from p-coumarate in the engineered P. putida. (A) p-Coumarate catabolic pathway; (B) Isoprenol production by engineered P. putida (JPUB_019977). EZ-rich medium was used as the base medium by changing the carbon source to 1% or 2% p-coumarate. The blank EZ-rich medium without any carbon sources was used as a control. Error bars indicate one standard deviation of triplicates. The initial OD₆₀₀ of 0 h was set at 0.05. Isoprenol levels were not detectable at 0 h.

FIG. 7 Production of sesquiterpene in the engineered P. putida. (A) Epi-isozizaene synthesis pathway from glucose. (B-D) Production results by the engineered P. putida strains from 1% glucose. (B) Cell growth; (C) Epi-isozizaene production; (D) Glucose consumption. Error bars indicate one standard deviation of triplicates. FPP, farnesyl diphosphate; FPPS, farnesyl diphosphate synthase; EizS, epi-isozizacne synthase from Streptomyces coelicolor. Error bars indicate one standard deviation of triplicates.

FIG. 8 Comparison of carbon flux distribution between E. coli and P. putida. The published 13C-metabolic flux data was used to compare the difference in carbon flux. Number in black, carbon flux of E. coli [Gonzalez J E, Long C P, Antoniewicz M R. Comprehensive analysis of glucose and xylose metabolism in Escherichia coli under aerobic and anaerobic conditions by 13C metabolic flux analysis. Metabolic Engineering. 2017; 39:9-18.]; Number in red, carbon flux of P. putida [Nikel P I, Chavarría M, Fuhrer T, Sauer U, de Lorenzo V. Pseudomonas putida KT2440 Strain Metabolizes Glucose through a Cycle Formed by Enzymes of the Entner-Doudoroff, Embden-Meyerhof-Parnas, and Pentose Phosphate Pathways. Journal of Biological Chemistry. 2015; 290:25920-32]. PEP, phosphoenolpyruvate; Pry, pyruvate; AcCoA, acetyl-CoA; Cit, citrate, AKG, 2-ketoglutarate; Mal, malate; OAA, oxaloacetate.

FIG. 9 Comparison of isoprenol production with P. putida gene-knockout strains bearing JPUB_019918 plasmid in a microtiter plate. A 24-well microtiter plate was used for screening isoprenol production using the EZ-rich medium containing 1% glucose. Data was shown after 48-hour production. Error bars indicate one standard deviation of triplicates.

FIG. 10 Isoprenol production with P. putida ΔphaABC strain (JPUB_019964) bearing JPUB_019925 plasmid in a shake flask using EZ-rich medium containing 2% glucose. Error bars indicate one standard deviation of triplicates.

FIG. 11 Isoprenol production by JPUB_019977 strain in M9 minimal medium supplemented with different concentrations of L-Glu. Error bars indicate one standard deviation of triplicates.

FIG. 12 Investigation of isoprenol consumption for P. putida Acre strain (JPUB_019978). Isoprenol consumption was performed in M9 minimal medium containing 1% glucose with or without 0.6 mM L-Glu supplement. (A) Cell growth represented by OD₆₀₀; (B) Isoprenol consumption; (C) Glucose consumption. Error bars indicate one standard deviation of triplicates.

FIG. 13 Isoprenol production with cre overexpression by P. putida ΔphaABC strain (JPUB_019964) with plasmid JPUB_019949 from 2% glucose. Error bars indicate one standard deviation of triplicates.

FIG. 14 Isoprenol consumption and production in P. putida ΔphaABC ΔPP_2675 strain (JPUB_019965). (A) Isoprenol consumption in M9 minimal medium and EZ-rich medium containing 1% glucose or no glucose, respectively. Results verified that the deletion of PP_2675 prevented isoprenol from self-consumption. (B) Isoprenol production in M9 minimal medium and EZ-rich medium containing 2% glucose using JPUB_019925 plasmid. The deletion of PP_2675 did not improve isoprenol titer. Error bars indicate one standard deviation of triplicates.

FIG. 15 Production of monoterpene in the engineered P. putida. (A) Monoterpene synthesis pathway from glucose. (B-D) Production results by the engineered P. putida strains from 1% glucose. (B) Cell growth. The cell growth of the 1,8-cincole-producing strain was slower than the limonene-producing strain (C) Monoterpene production. The highest limonene production was 49 mg/L after 24 h, and the highest 1,8-cincole production was 6 mg/L after 72 h. (D) Glucose consumption. The 1,8-cineole-producing strain remained at 8.6 g/L glucose after 24 h, which might explain the slower cell growth. Error bars indicate one standard deviation of triplicates. GPP, geranyl diphosphate; GPPS, geranyl diphosphate synthase; LS, limonene synthase from Mentha spicata. CS, 1,8-cineole synthase from Streptomyces clavuligerus. Error bars indicate one standard deviation of triplicates.

FIG. 16 Targeted proteomics of IPP-bypass MVA pathway in isoprenol production. Targeted proteomics was conducted as described previously [Mendez-Perez D, Alonso-Gutierrez J, Hu Q, Molinas M, Baidoo E E K, Wang G, et al. Production of jet fuel precursor monoterpenoids from engineered Escherichia coli. Biotechnology and Bioengineering. John Wiley & Sons, Ltd; 2017; 114:1703-12]. The results confirmed heterologous gene expression of the MVA pathway in P. putida strain JPUB_019968 during isoprenol production. Error bars indicate one standard deviation of triplicates.

FIG. 17. Genome-scale metabolic and pathway engineering for production of the sustainable aviation fuel (SAF) precursor, isoprenol, in Pseudomonas putida.

FIG. 18. Metabolic pathways for isoprenol production using IPP bypass in P. putida KT2440. The gene knockout targets identified from genome-scale metabolic modeling are shown in blue. The heterologous genes for the IPP-bypass mevalonate pathway are shown in green.

FIG. 19. Engineering gene knockout mutants for experimental validation of genome-scale metabolic modeling predictions. a The flowchart of gene knockout strain development; b Isoprenol production and cell growth of constructed gene knockout strains after 48 hours using EZ rich medium. Data was obtained from three biological replicates and error bars represent standard deviation.

FIG. 20. Pathway optimization in EZ Rich medium. a Schematic diagram of plasmids (Table 6) used in this study. b Isoprenol titer obtained at 48 hr from strains transformed with plasmids shown in a. c Cell density (OD₆₀₀) from strains shown in b measured at 48 h. Data were obtained from three biological replicates and error bars represent standard deviation. Asterisks indicate statistical significance by Student's t-test (**, 0.001<P<0.01; *** 0.0001<P<0.001; **** P<0.0001).

FIG. 21. a Isoprenol production and b growth in M9 glucose minimal medium using the optimized isoprenol production plasmid (pIY670, Table 6) after double adaptation. Data were obtained from three biological replicates and error bars represent standard deviation.

FIG. 22. Time-course profiles of the engineered P. putida KT2440 strains in M9 glucose minimal medium. a Growth, glucose consumption, and isoprenol production. Data were obtained from three biological replicates and error bars represent standard deviation. b Specific rates estimated using the first three time points (0 hr, 6 hr and 12 hr) during exponential growth. Error bars represent the estimated standard error.

FIG. 23. Flux variability analysis for the 6 different P. putida genotypes/strains with heterologous isoprenol production pathway (pIY670). a The central metabolic map is shown with reactions of interest highlighted and the heterologous isoprenol production pathway in green. b Flux span normalized to glucose uptake rate for selected reactions.

FIG. 24. Growth and isoprenol production in fed-batch mode. a optical density (OD₆₀₀) and b production of isoprenol by IY1245 (wild type), IY1262 (ΔphaABC ΔmvaB ΔhbdH ΔldhA), IY1452 (ΔphaABC ΔmvaB ΔhbdH ΔldhA ΔPP_2675), and IY1485 (ΔphaABC ΔmvaB ΔhbdH ΔldhA ΔPP_2675 ΔgacA). The fed-batch productions were performed in the 2 L biorcactors including M9 defined medium at triplicate. Error bars represent standard deviation.

FIG. 25. Isoprenol production and growth using hydrolysate. a production of isoprenol by IY1452 and b optical density (OD₆₀₀). The productions of isoprenol and growth were evaluated in the culture tubes including 5 mL modified M9 minimal medium with varying concentrations of hydrolysate at triplicate. Error bars represent standard deviation.

FIG. 26: Comparison of isoprenol production for multiple gene knockout strains. Time-course production of XW17 to XW19 strains in EZ rich media. Glucose, isoprenol, OD₆₀₀, and organic acids were measured. Data were obtained from three biological replicates and error bars represent standard deviation.

FIG. 27: Correlation between isoprenol production and cell growth of XW11 to XW19 strains in EZ rich media. Error bars represent standard deviation from three biological replicates.

FIG. 28: Fluorescence level of red fluorescent protein expressed under (A) PAllacO-1 and (B) P_{BAD} inducible promoter. Cultures were induced with different concentrations of IPTG or L-Arabinose, 2 hr after inoculation. Fluorescence level from at least 50000 cells was measured using a BD C6 Accuri flow cytometer with FL-4 detector at 24 hr. Error bars represent standard deviation of three biological replicates.

FIG. 29: Targeted proteomics of isoprenol production pathway. Data were obtained from three biological replicates and error bars represent standard deviation.

FIG. 30: Cell growth, glucose consumption, and isoprenol production/degradation from time-course experiment in M9 medium by IY1262 (ΔphaABC ΔmvaBΔhbdHΔldhA), IY1200 (ΔphaABCΔmvaBΔhbdHΔPP_2675), and IY1452 (ΔphaABCΔmvaBΔhbdHΔldhAΔPP_2675). Data were obtained from three biological replicates and error bars represent standard deviation.

FIG. 31: Flux variability analysis (FVA) for P. putida KT2440 ΔphaABC ΔmvaBΔhbdHΔldhAΔ2675 compared to WT flux span distribution normalized to glucose consumption.

FIG. 32: Subsystem-wise distribution of reactions that had a substantial fold change in flux for P. putida KT2440 ΔphaABCΔmvaBΔhbdHΔldhAΔ2675 compared to WT.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The term “host cell” is used to describe the genetically modified Pseudomonas cell.

The term “heterologous” as used herein refers to a material, or nucleotide or amino acid sequence, that is found in or is linked to another material, or nucleotide or amino acid sequence, wherein the materials, or nucleotide or amino acid sequences, are foreign to each other (i.e., not found or linked together in nature).

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “promoter,” as used herein, refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon (i.e., including the 5′ untranslated region of the mRNA, typically comprising 100-200 bp). Most often the core promoter sequences lie within 1-2 kb of the translation start site, more often within 1 kbp and often within 500 bp of the translation start site. By convention, the promoter sequence is usually provided as the sequence on the coding strand of the gene it controls. In the context of this application, a promoter is typically referred to by the name of the gene for which it naturally regulates expression. A promoter used in an expression construct of the invention is referred to by the name of the gene. Reference to a promoter by name includes a wildtype, native promoter as well as variants of the promoter that retain the ability to induce expression. Reference to a promoter by name is not restricted to a particular species, but also encompasses a promoter from a corresponding gene in other species.

A polynucleotide is “heterologous” to a host cell or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “operatively linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some embodiments, the Pseudomonas cell comprises a nucleic acid encoding the one or more enzymes operatively linked to a promoter capable of expressing the one or more enzymes in the Pseudomonas cell. In some embodiments, the encoding of the one or more enzymes to the nucleic acid is codon optimized to the Pseudomonas cell. In some embodiments, the nucleic acid is vector or replicon that can stably reside in the Pseudomonas cell. In some embodiments, the nucleic acid is stably integrated into the chromosome of the Pseudomonas cell.

In some embodiments, the providing step (a) comprises introducing a nucleic acid encoding the one or more enzymes operatively linked to a promoter capable of expressing the one or more enzymes in the Pseudomonas cell.

The present invention provides for a method for constructing a genetically modified Pseudomonas cell of the present invention, comprising (a) introducing a nucleic acid encoding the one or more enzymes operatively linked to a promoter capable of expressing the one or more enzymes in the Pseudomonas cell.

One can modify the expression of a gene encoding any of the enzymes taught herein by a variety of methods in accordance with the methods of the invention. Those skilled in the art would recognize that increasing gene copy number, ribosome binding site strength, promoter strength, and various transcriptional regulators can be employed to alter an enzyme expression level.

In some embodiments, the Pseudomonas cells are genetically modified in that heterologous nucleic acid have been introduced into the Pseudomonas cells, and as such the genetically modified Pseudomonas cells do not occur in nature. The suitable Pseudomonas cell is one capable of expressing a nucleic acid construct encoding one or more enzymes described herein. The gene(s) encoding the enzyme(s) may be heterologous to the Pseudomonas cell or the gene may be native to the Pseudomonas cell but is operatively linked to a heterologous promoter and one or more control regions which result in a higher expression of the gene in the Pseudomonas cell.

The enzyme can be native or heterologous to the Pseudomonas cell. Where the enzyme is native to the Pseudomonas cell, the Pseudomonas cell is genetically modified to modulate expression of the enzyme. This modification can involve the modification of the chromosomal gene encoding the enzyme in the Pseudomonas cell or a nucleic acid construct encoding the gene of the enzyme is introduced into the Pseudomonas cell. One of the effects of the modification is the expression of the enzyme is modulated in the Pseudomonas cell, such as the increased expression of the enzyme in the Pseudomonas cell as compared to the expression of the enzyme in an unmodified Pseudomonas cell.

In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida cell.

The biomass can be obtained from one or more feedstock, such as softwood feedstock, hardwood feedstock, grass feedstock, and/or agricultural feedstock, or a mixture thereof.

Softwood feedstocks include, but are not limited to, Araucaria (e.g. A. cunninghamii, A. angustifolia, A. araucana); softwood Cedar (e.g. Juniperus virginiana, Thuja plicata, Thuja occidentalis, Chamaecyparis thyoides Callitropsis nootkatensis); Cypress (e.g. Chamaecyparis, Cupressus Taxodium, Cupressus arizonica, Taxodium distichum, Chamaecyparis obtusa, Chamaecyparis lawsoniana, Cupressus semperviren); Rocky Mountain Douglas fir; European Yew; Fir (e.g. Abies balsamea, Abies alba, Abies procera, Abies amabilis); Hemlock (e.g. Tsuga canadensis, Tsuga mertensiana, Tsuga heterophylla); Kauri; Kaya; Larch (e.g. Larix decidua, Larix kaempferi, Larix laricina, Larix occidentalis); Pine (e.g. Pinus nigra, Pinus banksiana, Pinus contorta, Pinus radiata, Pinus ponderosa, Pinus resinosa, Pinus sylvestris, Pinus strobus, Pinus monticola, Pinus lambertiana, Pinus taeda, Pinus palustris, Pinus rigida, Pinus echinata); Redwood; Rimu; Spruce (e.g. Picea abies, Picea mariana, Picea rubens, Picea sitchensis, Picea glauca); Sugi; and combinations/hybrids thereof.

For example, softwood feedstocks which may be used herein include cedar; fir; pine; spruce; and combinations thereof. The softwood feedstocks for the present invention may be selected from loblolly pine (Pinus tacda), radiata pine, jack pine, spruce (e.g., white, interior, black), Douglas fir, Pinus silvestris, Picea abies, and combinations/hybrids thereof. The softwood feedstocks for the present invention may be selected from pine (e.g. Pinus radiata, Pinus tacda); spruce; and combinations/hybrids thereof.

Hardwood feedstocks include, but are not limited to, Acacia; Afzelia; Synsepalum duloificum; Albizia; Alder (e.g. Alnus glutinosa, Alnus rubra); Applewood; Arbutus; Ash (e.g. F. nigra, F. quadrangulata, F. excelsior, F. pennsylvanica lanceolata, F. latifolia, F. profunda, F. americana); Aspen (e.g. P. grandidentata, P. tremula, P. tremuloides); Australian Red Cedar (Toona ciliata); Ayna (Distemonanthus benthamianus); Balsa (Ochroma pyramidale); Basswood (e.g. T. americana, T. heterophylla); Beech (e.g. F. sylvatica, F. grandifolia); Birch; (e.g. Betula populifolia, B. nigra, B. papyrifera, B. lenta, B. alleghaniensis/B. lutea, B. pendula, B. pubescens); Blackbean; Blackwood; Bocote; Boxelder; Boxwood; Brazilwood; Bubing a; Buckeye (e.g. Aesculus hippocastanum, Aesculus glabra, Aesculus flava/Aesculus octandra); Butternut; Catalpa; Chemy (e.g. Prunus serotina, Prunus pennsylvanica, Prunus avium); Crabwood; Chestnut; Coachwood; Cocobolo; Corkwood; Cottonwood (e.g. Populus balsamifera, Populus deltoides, Populus sargentii, Populus heterophylla); Cucumbertree; Dogwood (e.g. Cornus florida, Cornus nuttallii); Ebony (e.g. Diospyros kurzii, Diospyros melanida, Diospyros crassiflora); Elm (e.g. Ulmus americana, Ulmus procera, Ulmus thomasii, Ulmus rubra, Ulmus glabra); Eucalyptus; Greenheart; Grenadilla; Gum (e.g. Nyssa sylvatica, Eucalyptus globulus, Liquidambar styraciflua, Nyssa aquatica); Hickory (e.g. Carya alba, Carya glabra, Carya ovata, Carya laciniosa); Hornbeam; Hophornbeam; Ipê; Iroko; Ironwood (e.g. Bangkirai, Carpinus caroliniana, Casuarina equisetifolia, Choricbangarpia subargentea, Copaifera spp., Eusideroxylon zwageri, Guajacum officinale, Guajacum sanctum, Hopea odorata, Ipe, Krugiodendronferreum, Lyonothamnus lyonii (L. floribundus), Mesua ferrea, Olea spp., Olneya tesota, Ostrya virginiana, Parrotia persica, Tabebuia serratifolia); Jacarandá; Jotoba; Lacewood; Laurel; Limba; Lignum vitae; Locust (e.g. Robinia pseudacacia, Gleditsia triacanthos); Mahogany; Maple (e.g. Acer saccharum, Acer nigrum, Acer negundo, Acer rubrum, Acer saccharinum, Acer pseudoplatanus); Meranti; Mpingo; Oak (e.g. Quercus macrocarpa, Quercus alba, Quercus stellata, Quercus bicolor, Quercus virginiana, Quercus michauxii, Quercus prinus, Quercus muhlenbergii, Quercus chrysolepis, Quercus lyrata, Quercus robur, Quercus petraea, Quercus rubra, Quercus velutina, Quercus laurifolia, Quercus falcata, Quercus nigra, Quercus phellos, Quercus texana); Obeche; Okoumé; Oregon Myrtle; California Bay Laurel; Pear; Poplar (e.g. P. balsamifera, P. nigra, Hybrid Poplar (Populusxcanadensis)); Ramin; Red cedar; Rosewood; Sal; Sandalwood; Sassafras; Satinwood; Silky Oak; Silver Wattle; Snakewood; Sourwood; Spanish cedar; American sycamore; Teak; Walnut (e.g. Juglans nigra, Juglans regia); Willow (e.g. Salix nigra, Salix alba); Yellow poplar (Liriodendron tulipifera); Bamboo; Palmwood; and combinations/hybrids thereof.

For example, hardwood feedstocks for the present invention may be selected from Acacia, Aspen, Beech, Eucalyptus, Maple, Birch, Gum, Oak, Poplar, and combinations/hybrids thereof. The hardwood feedstocks for the present invention may be selected from Populus spp. (e.g. Populus tremuloides), Eucalyptus spp. (e.g. Eucalyptus globulus), Acacia spp. (e.g. Acacia dealbata), and combinations thereof.

Grass feedstocks include, but are not limited to, C₄ or C₃ grasses, e.g. Switchgrass, Indiangrass, Big Bluestem, Little Bluestem, Canada Wildrye, Virginia Wildrye, and Goldenrod wildflowers, etc, amongst other species known in the art.

Agricultural feedstocks include, but are not limited to, agricultural byproducts such as husks, stovers, foliage, and the like. Such agricultural byproducts can be derived from crops for human consumption, animal consumption, or other non-consumption purposes. Such crops can be corps such as corn, wheat, rice, soybeans, hay, potatoes, cotton, or sugarcane.

The feedstock can arise from the harvesting of crops from the following practices: intercropping, mixed intercropping, row cropping, relay cropping, and the like.

Other objects, features, and advantages of the present invention will be apparent to one of skill in the art from the following detailed description and figures.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Engineering Isoprenoids Production in Metabolically Versatile Microbial Host Pseudomonas putida

With the increasing need for microbial bioproduction to replace petrochemicals, it is critical to develop a new industrial microbial workhorse that improves the conversion of lignocellulosic carbon to biofuels and bioproducts in an economically feasible manner. Pseudomonas putida KT2440 is a promising microbial host due to its capability to grow on a broad range of carbon sources and its high tolerance to xenobiotics. In this study, we engineered P. putida KT2440 to produce isoprenoids, a vast category of compounds that provide routes to many petrochemical replacements. A heterologous mevalonate (MVA) pathway was engineered to produce potential biofuels isoprenol (C₅) and epi-isozizaene (C₁₅) for the first time in P. putida. We compared the difference of three different isoprenoid pathways in P. putida on isoprenol production and achieved 104 mg/L of isoprenol production in a batch flask experiment through optimization of the strain. As P. putida can natively consume isoprenol, we investigated how to prevent this self-consumption. We discovered that supplementing L-glutamate in the medium can effectively prevent isoprenol consumption in P. putida and metabolomics analysis showed an insufficient energy availability and an imbalanced redox status during isoprenol degradation. We also showed that the engineered P. putida strain can produce isoprenol using aromatic substrates such as p-coumarate as the sole carbon source, and this result demonstrates that P. putida is a valuable microbial chassis for isoprenoids to achieve sustainable biofuel production from lignocellulosic biomass.

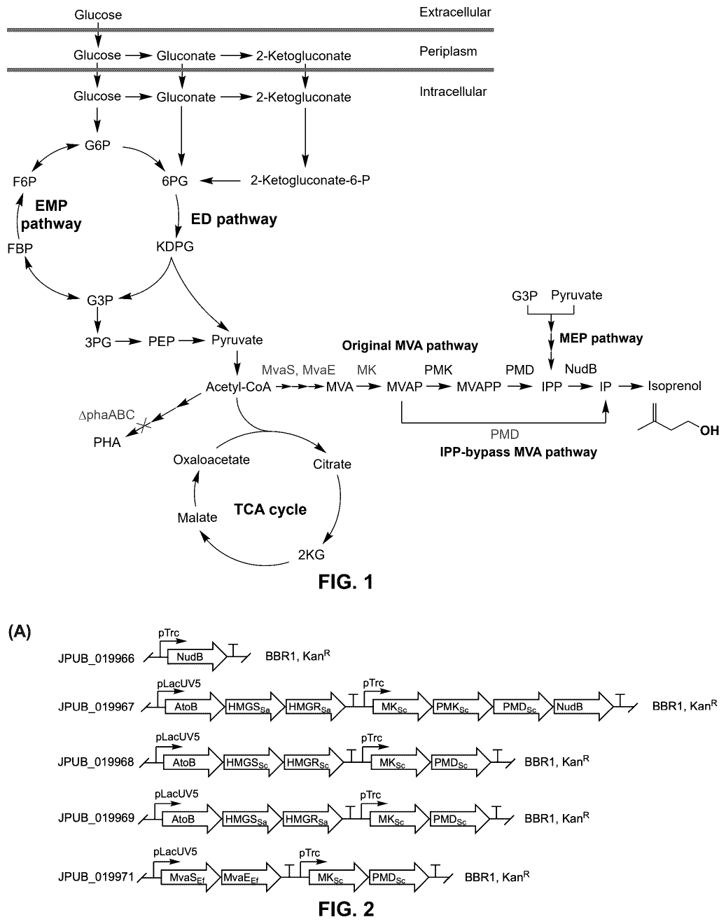

In this study, we engineered the heterologous MVA pathway in P. putida KT2440 to produce isoprenoids, including isoprenol (C₅) and epi-isozizaene (C₁₅). We compared the differences among the MEP, MVA, and IPP-bypass MVA pathways during isoprenol production (FIG. 1). Since isoprenol can be utilized as a carbon source by P. putida KT2440 [28], we investigated strategies to prevent isoprenol self-consumption. Metabolomics was performed to reveal the metabolic difference during isoprenol degradation. We also showed the engineered P. putida can produce isoprenol using p-coumarate as the sole carbon source. Our results showed that P. putida is a promising microbial chassis for isoprenoids production with the improved capability of carbon utilization from lignocellulosic biomass for biofuel production.

### 2. MATERIAL AND METHODS

### 2.1 Strains and Plasmid Construction

All strains and plasmids used in this study are listed in Table 1. Strains and plasmids along with their associated information have been deposited in the public version of the JBEI Registry (website for: public-registry.jbei.org; entries JPUB_019914 to JPUB_019988) and are available from the authors upon request. P. putida KT2440 was used for isoprenoid production, and E. coli DH5α was used for the general cloning.

TABLE 1
Strains and plasmids used in this example.

| | Description | Reference |
|---|---|---|
| Strains | | |
| JPUB_019964 | P. putida KT2440 deleted with the phaA-phaB-phaC gene | This study |
| (ΔphaABC) | cluster (PP_5003-PP_5005) | |
| JPUB_019965 | P. putida KT2440 ΔphaABC APP_2675 | This study |
| JPUB_019966 | P. putida KT2440 with pBbB1k-NudB | This study |
| JPUB_019967 | P. putida KT2440 with pBbB5k-MTSA-T1-MK_{sc}-PMK- | This study |
| | PMD_{sc}-NudB | |
| JPUB_019968 | P. putida KT2440 with pBbB5k-AtoB-HMGS_{sc}-HMGR_{sc}- | This study |
| | T1-MK_{sc}-PMD_{sc} | |
| JPUB_019969 | P. putida KT2440 with pBbB5k-AtoB-HMGSₛₐ-HMGRₛₐ- | This study |
| | T1-MK_{sc}-PMD_{sc} | |
| JPUB_019971 | P. putida KT2440 with pBbB5k-MvaS_{ef}-MvaE_{ef}-T1- | This study |
| | MK_{sc}-PMD_{sc} | |
| JPUB_019973 | P. putida ΔphaABC with pBbB5k-AtoB-HMGS_{sc}- | This study |
| | HMGR_{sc}-T1-MK_{sc}-PMD_{sc} | |
| JPUB_019974 | P. putida ΔphaABC with pBbB5k-MvaS_{ef} -MvaE_{ef}-T1- | This study |
| | MK_{sc}-PMD_{sc} | |
| JPUB_019975 | P. putida ΔphaABC with pBbB5k-MvaS_{ef}-MvaE_{ef}-T1- | This study |
| | MK_{sc}-PMD_{HKQ} | |
| JPUB_019976 | P. putida ΔphaABC with pBbB5k-MvaS_{ef}-MvaE_{ef}-T1- | This study |
| | MKₘₘ-PMD_{sc} | |
| JPUB_019977 | P. putida ΔphaABC with pBbB5k-MvaS_{ef}-MvaE_{ef}-T1- | This study |
| | MKₘₘ-PMD_{HKQ} | |
| JPUB_019978 | P. putida ΔphaABC deleted with the crc gene (PP_5292) | This study |
| JPUB_019986 | P. putida KT2440 with pBbB1k-EizS | This study |
| JPUB_019987 | P. putida KT2440 with pBbB5k-MTSA-T1-MK_{sc}-PMK- | This study |
| | PMD_{sc}-idi-ispA-T1-EizS | |
| JPUB_019988 | P. putida ΔphaABC with pBbB5k-MTSA-T1-MK_{sc}-PMK- | This study |
| | PMD_{sc}-idi-ispA-T1-EizS | |
| Plasmids | | |
| JPUB_019914 | pBbB1k-NudB | This study |
| JPUB_019916 | pBbB5k-MTSA-T1-MK_{sc}-PMK-PMD_{sc}-NudB | This study |
| JPUB_019918 | pBbB5k-AtoB-HMGS_{sc}-HMGR_{sc}-T1-MK_{sc}-PMD_{sc} | This study |
| JPUB_019970 | pBbB5k-AtoB-HMGSₛₐ-HMGRₛₐ -T1-MK_{sc}-PMD_{sc} | This study |
| JPUB_019920 | pBbB5k-MvaS_{ef}-MvaE_{ef}-T1-MK_{sc}-PMD_{sc} | This study |
| JPUB_019922 | pBbB5k-MvaS_{ef}-MvaE_{ef}-T1-MK_{sc}-PMD_{HKQ} | This study |
| JPUB_019923 | pBbB5k-MvaS_{ef}-MvaE_{ef}-T1-MKₘₘ-PMD_{sc} | This study |
| JPUB_019925 | pBbB5k-MvaS_{ef}-MvaE_{ef}-T1-MKₘₘ-PMD_{HKQ} | This study |
| JPUB_019933 | pBbB1k-EizS | This study |
| JPUB_019935 | pBbB5k-MTSA-T1-MK_{sc}-PMK-PMD_{sc}-idi-ispA-T1-EizS | This study |
| JPUB_019939 | pK18-ppc | This study |
| JPUB_019941 | pK18-pyc | This study |
| JPUB_019943 | pK18-phaABC | This study |
| JPUB_019945 | pK18-crc | This study |
| JPUB_018413 | pNQ30-PP_2675 | [28] |
| JPUB_019949 | pBbB5k-MvaS_{ef}-MvaE_{ef}-T1-MKₘₘ-PMD_{HKQ}-crc | This study |

Transformation of P. putida was performed by electroporation using a Bio-Rad (Bio-Rad Laboratories, Hercules, CA) MicroPulser preprogrammed EC3 setting (0.2 cm cuvettes with 50 μL cells, ˜5 ms pulse, 3.0 kV) [29]. LB medium and LB agar medium were used for cell outgrowth and colony selection at 30° C., respectively. Kanamycin (50 μg/mL) or gentamicin (30 μg/mL) was used as the selective antibiotics when needed. Gene knockout of P. putida was performed based on the homologous recombination followed by a suicide gene (sacB) counter-selection using modified pK18-mobSacB plasmids [30]. The genotypes of gene-knockout mutants were confirmed by colony PCR using specific primers, followed by DNA sequencing (GENEWIZ, South San Francisco, CA, USA).

2.2 Isoprenol Production in P. putida

An overview figure of typical process of isoprenol production and analysis is presented in the Supplementary information.

P. putida KT2440 strains bearing isoprenol pathway plasmids (Table 1) were used for isoprenol production. Starter cultures of all production strains were prepared by growing single colonies in LB medium containing 50 μg/mL kanamycin at 30° C. with 200-rpm shaking overnight. The starter cultures were diluted in 5 mL EZ-rich defined medium (Teknova, CA, USA) or M9 minimal medium [29], containing 10 g/L or 20 g/L glucose (1% or 2%, w/v), 25 μg/mL kanamycin in 50-mL culture tubes, and 0.5 mM IPTG was added to induce protein expression with OD₆₀₀ at 0.4-0.6. When strains were cultivated in a 24-well microtiter plate, 2 mL medium was used and the plate was sealed with a gas-permeable film (Sigma-Aldrich, St. Louis, MO). When strains were cultivated in a 250-mL shake flask, 50-mL medium was used. L-glutamate was supplemented into the minimal medium at the indicated concentration when needed. For isoprenol production using p-coumarate as the carbon source, 10 g/L or 20 g/L (1% or 2%, w/v) p-coumarate was used to replace glucose in the EZ-rich defined medium. The P. putida cultures were incubated in rotary shakers (200 rpm) at 30° C. for 48 hours.

### 2.3 Evaluation of Isoprenol Consumption

P. putida strains (Table 1) were used to investigate isoprenol consumption. Starter cultures were prepared by inoculating glycerol stocks in LB medium at 30° C. with 200-rpm shaking overnight. The starter cultures were diluted with OD₆₀₀ at 0.01 in 5 mL M9 minimal medium or EZ-rich defined medium (Teknova, CA, USA) containing 10 g/L glucose (1%, w/v) or no glucose (0%, w/v), added with 1 g/L isoprenol in 50-mL culture tubes. Amino acids (Table 2) were added individually into the M9 minimal medium at desirable concentrations when needed. The P. putida cultures were incubated in rotary shakers (200 rpm) at 30° C. for 48 hours. Blank media without strain inoculation were used in parallel to evaluate isoprenol evaporation loss.

### 2.4 Quantification of Isoprenol

The measurement and quantification of isoprenol were conducted by collecting 250 μL of cell culture and combining it with 250 μL of ethyl acetate containing 1-butanol (30 mg/L) as an internal standard. The mixture of ethyl acetate and cell culture was vigorously shaken for 15 min and subsequently centrifuged at 21,130 g for 3 min to separate the ethyl acetate phase from the aqueous phase. The ethyl acetate layer was collected and 1 μL was analyzed by gas chromatography-flame ionization detection (GC-FID, Thermo Focus GC) equipped with DB-WAX column (15 m, 0.32 mm inner diameter, 0.25 μm film thickness, Agilent, USA). The GC oven was programmed as follow: 40° C. to 100° C. at 15° C./min, 100° C. to 230° C. at 40° C./min, held at 230° C. for 2 min. The inlet temperature was 200° C.

### 2.5 Production and Quantification of Epi-Isozizaene

P. putida KT2440 bearing the pathway plasmid (Table 1) was used for epi-isozizaene production. Starter cultures of all production strains were prepared by growing single colonies in LB medium containing 50 μg/mL kanamycin at 30° C. with 200-rpm shaking overnight. The starter cultures were diluted in a 5 mL EZ-rich defined medium (Teknova, CA, USA) containing 10 g/L glucose (1%, w/v), 25 μg/mL kanamycin in 50-mL culture tubes. 0.5 mM IPTG was added to induce protein expression with OD₆₀₀ at 0.4-0.6, and 0.5 mL nonane (10%, v/v) was added as a solvent overlay. The P. putida cultures were incubated in rotary shakers (200 rpm) at 30° C. for 72 hours.

For epi-isozizaene measurement, the solvent overlay was sampled and centrifuged at 21,130 g for 3 min. The overlay layer was collected and diluted with ethyl acetate containing 5 mg/L guaiazulene as the internal standard. 1 μL was analyzed by Agilent GC-MS equipped with HP-5 column (Agilent, USA). The GC oven was programmed from 40° C. (held for 3 min) to 295° C. at 15° C./min. The concentration of epi-isozizaene was estimated using the TIC areas with alternative standard (−)-trans-caryophyllene as described in a previous study [31].

### 2.6 Quantification of Metabolites

The concentrations of glucose and organic acids from the culture were measured with an Agilent 1100 Series HPLC system, equipped with an Agilent 1200 Series refractive index detector (RID) (Agilent Technologies, CA) and Aminex HPX-87H ion-exclusion column as described in a previous study [32]. The quantification of glucose and organic acids was calibrated with authentic standards.

For metabolomics analysis, 1.5 mL cell culture was collected at 24 and 48 hours and centrifuged at 13,000 g for 1 min at room temperature. The cell pellet was quenched with 250 μL methanol, vortexed, and stored at −20° C. For sample preparation, 250 μL water was added to the methanol lysate and mix thoroughly. Centrifuge the methanol/water lysate at 13,000 g for 10 min at 4° C. The supernatant was filtered by a Millipore Amicon Ultra 3 kDa cut-off filter (Billerica, MA) at 13,000 g at −2° C. for 30-60 min until most of the sample has been filtered. The intracellular metabolite concentrations were quantified by liquid chromatography and mass spectrometry (LC-MS) methods as previously described by Baidoo et al. (with reference to note 6).

Monoterpene Production and Quantification in P. putida

P. putida KT2440 strains bearing monoterpene pathway plasmids (Table 3) were used for limonene or 1,8-cineole production. Starter cultures of all production strains were prepared by growing single colonies in LB medium containing 50 μg/mL kanamycin at 30° C. with 200-rpm shaking overnight. The starter cultures were diluted in 5 mL EZ-rich defined medium (Teknova, CA, USA), containing 10 g/L glucose (1%, w/v), 25 μg/mL kanamycin in 50-mL culture tubes, and 0.5 mM IPTG was added to induce protein expression with OD₆₀₀ at 0.4-0.6. 0.5 mL dodecane (10%, v/v) was added as a solvent overlay. The P. putida cultures were incubated in rotary shakers (200 rpm) at 30° C. for 48 hours. The measurement and quantification of limonene and 1,8-cineole were conducted as described previously [Wang X, Pereira J H, Tsutakawa S, Fang X, Adams P D, Mukhopadhyay A, et al. Efficient production of oxidized terpenoids via engineering fusion proteins of terpene synthase and cytochrome P450. Metabolic Engineering. 2021; 64:41-51.].

TABLE 2
Identified 8 amino acids from the EZ-rich medium and
their working concentrations.

| No. | Amino acid | Concentration |
|---|---|---|
| 1 | L-Arginine HCl (L-Arg) | 5.2 mM |
| 2 | L-Glutamic acid, potassium salt (L-Glu) | 0.6 mM |
| 3 | L-Glutamine (L-Gln) | 0.6 mM |
| 4 | L-Glycine (L-Gly) | 0.8 mM |
| 5 | L-Serine (L-Ser) | 10 mM |
| 6 | L-Valine (L-Val) | 0.6 mM |
| 7 | L-Leucine (L-Leu) | 0.8 mM |
| 8 | L-Alanine (L-Ala) | 0.8 mM |

TABLE 3
Strains and plasmids used in monoterpene production.

| | Description | Reference |
|---|---|---|
| Strains | | |
| JPUB_019980 | P. putida KT2440 with pBbB5k-MTSA- | This study |
| | T1-MK_{sc}-PMK-PMD_{sc}-idi-T1-trGPPS-LS | |
| JPUB_019984 | P. putida KT2440 with pBbB5k-MTSA- | This study |
| | T1-MK_{sc}-PMK-PMD_{sc}-idi-T1-trGPPS-CS | |
| Plasmids | | |
| JPUB_019930 | pBbB5k-MTSA-T1-MK_{sc}-PMK-PMD_{sc}- | This study |
| | idi-T1-trGPPS-LS | |
| JPUB_019932 | pBbB5k-MTSA-T1-MK_{sc}-PMK-PMD_{sc}- | This study |
| | idi-T1-trGPPS-CS | |

### 3. RESULTS

3.1 Engineering P. putida for Isoprenol Production

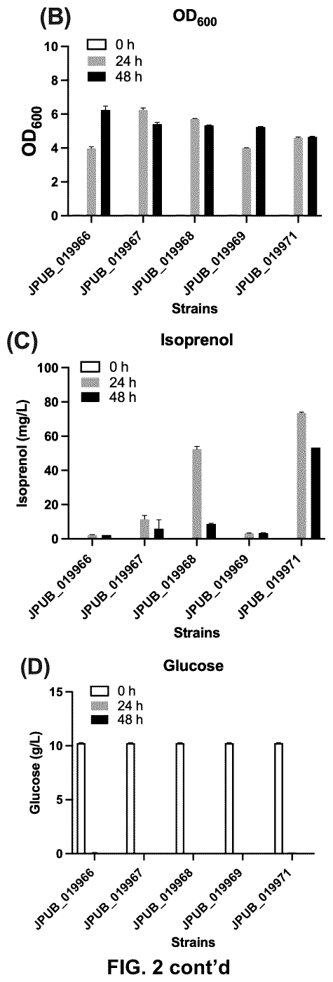

P. putida natively possesses the MEP pathway for isoprenoids biosynthesis. To produce isoprenol in P. putida, we first attempted to use the endogenous MEP pathway and overexpressed the E. coli dihydroneopterin triphosphate diphosphatase (NudB) that has a promiscuous activity to catalyze the conversion of IPP to IP which is hydrolyzed to isoprenol by endogenous phosphatases [22]. In this case, P. putida KT2440 was transformed with a high-copy plasmid pBbBlk-NudB (Table 1) using a modified broad host range replication origin BBR1 [34] and a Trc promoter which works both in E. coli and P. putida. The engineered P. putida strain (JPUB_019966, Table 1, FIG. 2, A) could produce a low level of isoprenol at 2 mg/L after 48 hours from 1% glucose (FIG. 2, C).

We then engineered a heterologous MVA pathway, which has shown high isoprenol production in E. coli [14]. To construct the MVA pathway, two operons were used to express the MVA pathway genes onto the plasmid backbone of pBbB5k. The expression of the top portion of the MVA pathway (AtoB, HMGS, HMGR) was driven by a LacUV5 promoter, and the expression of the bottom portion enzymes (MK, PMK, PMD) as well as NudB were driven by a Trc promoter. The resulting engineered P. putida strain (JPUB_019967, Table 1, FIG. 2, A) produced up to 12 mg/L isoprenol at 24 hours and the titer decreased at a later time point (FIG. 2, C).

Finally we engineered the IPP-bypass MVA pathway to compare the isoprenol production by using the promiscuous activity of PMD in P. putida. Three different MVA pathway top-portion operons (MevT, MTSA, and MvaES) were studied, which the HMGS and HMGR genes are from Saccharomyces cerevisiae, Staphylococcus aureus, and Enterococcus faecalis, respectively (JPUB_019968 to JPUB_019971, Table 1, FIG. 2, A). Results showed that the highest isoprenol production (up to 74 mg/L after 24 hours) was observed in the strain with the MvaES top portion operon (FIG. 2, C). Compared with the strain using the original MVA pathway, strains with the IPP-bypass MVA pathways (except the one using the MTSA top portion operon) showed a 4 to 6-fold increase of isoprenol production. This suggests the IPP-bypass MVA pathway can be used in P. putida for isoprenol production and it shows higher efficiency than the original MVA pathway or the endogenous MEP pathway.

3.2 Optimization of Isoprenol Production in P. putida.

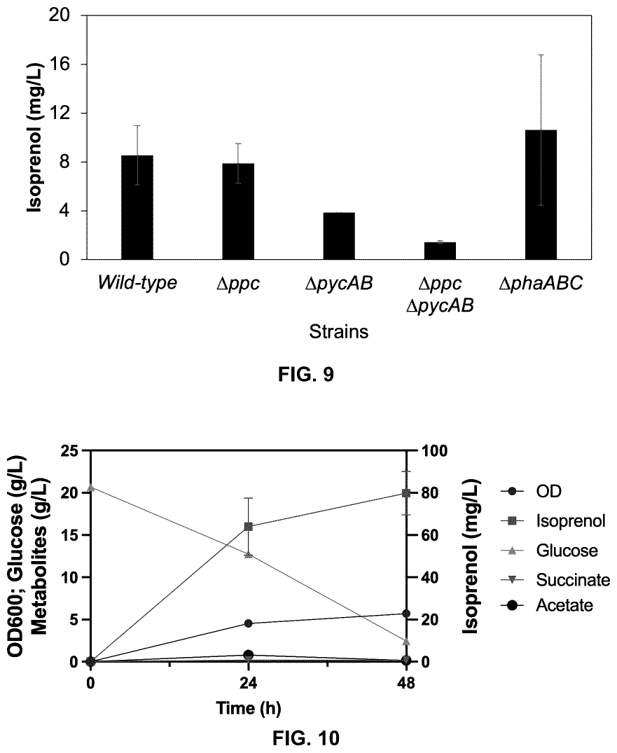

Given that E. coli has shown much higher isoprenol production than what we achieved in P. putida, we compared the metabolic difference between P. putida and E. coli to identify limiting steps and target them to optimize isoprenol production in P. putida. We used the published 13C-metabolic flux data of P. putida and E. coli for the comparison (FIG. 8). Interestingly, we found P. putida derived 3-fold more carbon flux from acetyl-CoA to TCA cycle compared with E. coli, which indicated less acetyl-CoA availability in P. putida for isoprenol production. Another difference is P. putida possesses both phosphoenolpyruvate carboxylase (ppc) and pyruvate carboxylase (pyc) that can direct 2.8-fold more flux from glycolysis to the TCA cycle, whereas the pyc gene does not exist in E. coli. In addition, P. putida can naturally synthesize polyhydroxyalkanoate (PHA) from acetyl-CoA as a carbon sink [36]. Therefore, we attempted to knockout ppc, pycAB, and phaABC (PHA synthase) genes to improve acetyl-CoA pool and isoprenol production in P. putida. As the Supplementary FIG. 9 showed, the knockout of ppc and pycAB genes did not improve isoprenol production compared with the wild-type strain. The double knockout of ppc and pycAB genes produced an even lower amount of isoprenol. However, the deletion of phaABC genes increased 24% of isoprenol production compared with the wild-type strain during the screening in a microtiter plate, which suggests they are promising targets.

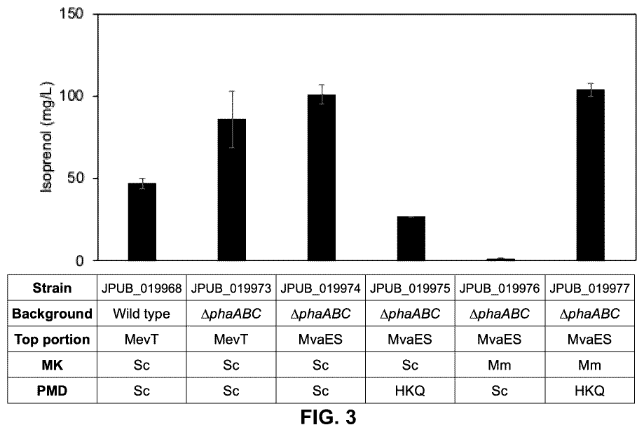

On the other hand, we noticed that the production results in the previous section showed decreased isoprenol levels and depleted glucose after 24 hours (FIG. 9, C). This indicated 1% glucose concentration might be insufficient to support a 48-hour production process. Thus, we increased the initial glucose concentration to 2%, and the IPP-bypass MVA pathway with the MevT operon improved the isoprenol production level from 9 mg/L to 47 mg/L after 48 hours (FIG. 3). When using the ΔphaABC strain, isoprenol production was further increased to 86 mg/L from 2% glucose at 48 hours (FIG. 3). By applying this new condition (ΔphaABC strain+2% glucose) to the best producing pathway (IPP-bypass_MvaES), the engineered P. putida strain (JPUB_019974, Table 1) reached 101 mg/L isoprenol production after 48 hours (FIG. 3), which was a ˜2-fold improvement of isoprenol production from the starting conditions (53 mg/L from the wild-type strain and 1% glucose).

Given that MK and PMD are key steps to converting MVA to isoprenol, we also tested different combinations of the MK-PMD gene cassettes to optimize isoprenol production. Based on previous results in E. coli [24], we selected two efficient enzyme versions, MK_{Mm} (MK from Methanosarcina mazei) and PMD_{HKQ} (a mutant of PMDse containing three mutations [37]) to construct four combinations of the MK-PMD cassette. Results showed that the strain with MK_{Mm}-PMD_{HKQ} (JPUB_019977, Table 1) produced the highest isoprenol at 104 mg/L after 48 hours from 2% glucose in a culture tube (FIG. 3). More production details were studied by culturing this best producer in a shake flask. As the Supplementary FIG. 10 shows, glucose was not fully depleted and ˜2.5 g/L of residual glucose was detected in the culture after 48 hours, which suggested the initial glucose concentration at 2% was sufficient in supporting a 48-hour production. No significant amounts of organic acids were detected as fermentative byproducts except the small amount of acetate (0.8 g/L) and succinate (0.2 g/L) observed only at 24 hours. While the isoprenol titer was lower in the shake flask (80 mg/L), it might be attributed to potentially faster isoprenol evaporation in the flask than in the culture tube. Unlike the top-portion MVA pathway, changing MK-PMD genes did not significantly improve isoprenol production. Collectively, we engineered the IPP-bypass MVA pathway in P. putida KT2440 for isoprenol production and achieved the highest production titer from glucose at up to 104 mg/L (c.f. the maximum theoretical yield from glucose is 0.319 g/g glucose [38]).

3.3 Investigation of Isoprenol Consumption in P. putida.

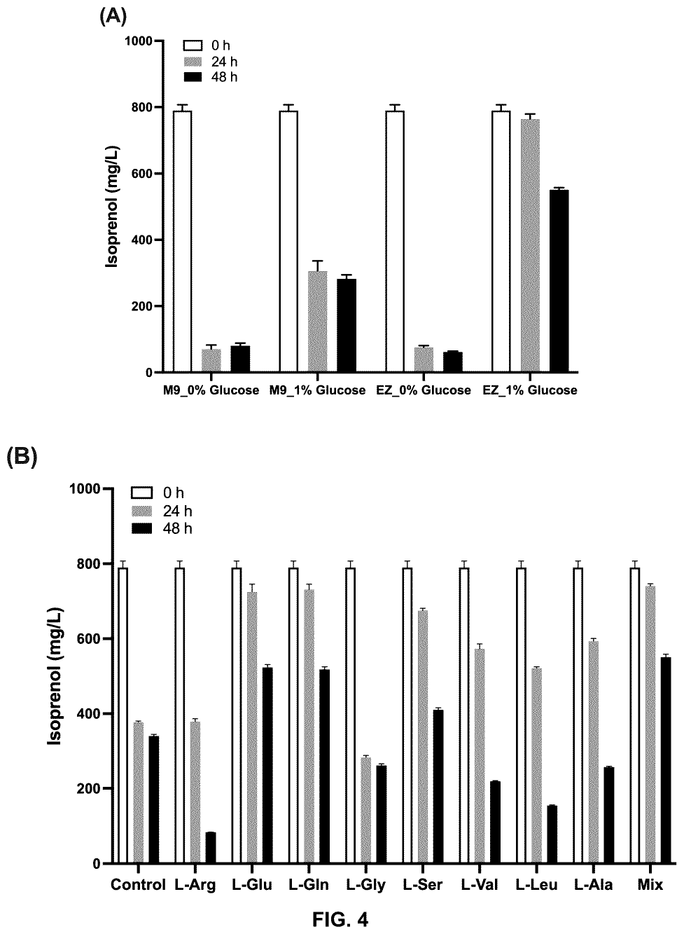

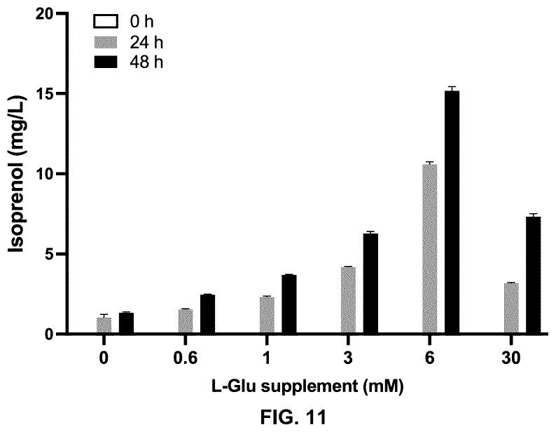

While the above isoprenol production was performed in the EZ-rich defined medium, it is also important to perform the production in the minimal medium, which is more frequently used for bioreactor fermentation and metabolic flux analysis [24]. Using the highest isoprenol producer (JPUB_019977, Table 1) from the EZ-rich defined medium, we tested isoprenol production in M9 minimal medium but observed low levels of isoprenol (˜1 mg/L) after 48 hours from 2% glucose (FIG. 11). Since P. putida has shown the capability of utilizing isoprenol as a carbon source [28], this urged us to investigate the difference between the two media that were used for isoprenol production. We first compared isoprenol consumption in the M9 minimal medium and EZ-rich medium. It was observed that the addition of glucose could help to preserve isoprenol in the medium, and the consumption was significantly slower in the EZ-rich medium when glucose is present (5 mg/L/hour) than in the M9 minimal medium (11 mg/L/hour) in 48 hours (FIG. 4, A).

To find out which other component of the EZ-rich medium contributed to slowing down the isoprenol consumption, we compared the recipes of two media and identified 8 amino acids that are present at a higher concentration in the EZ-rich medium formulation (Table 2). By supplementing these 8 amino acids individually into the M9 minimal medium at the same concentration used in the EZ-rich medium, surprisingly, we found that the addition of L-glutamate (L-Glu) or L-glutamine (L-Gln) preserved isoprenol to a similar level that was observed in the EZ-rich medium (FIG. 4, B). We chose L-Glu as the supplement to investigate isoprenol production in the minimal medium and observed that the addition of 6 mM L-Glu resulted the highest isoprenol production level to 15 mg/L after 48 hours (Supplementary FIG. 1-S4), which is nearly a 15-fold increase compared with the previous level without any supplements (˜1 mg/L).

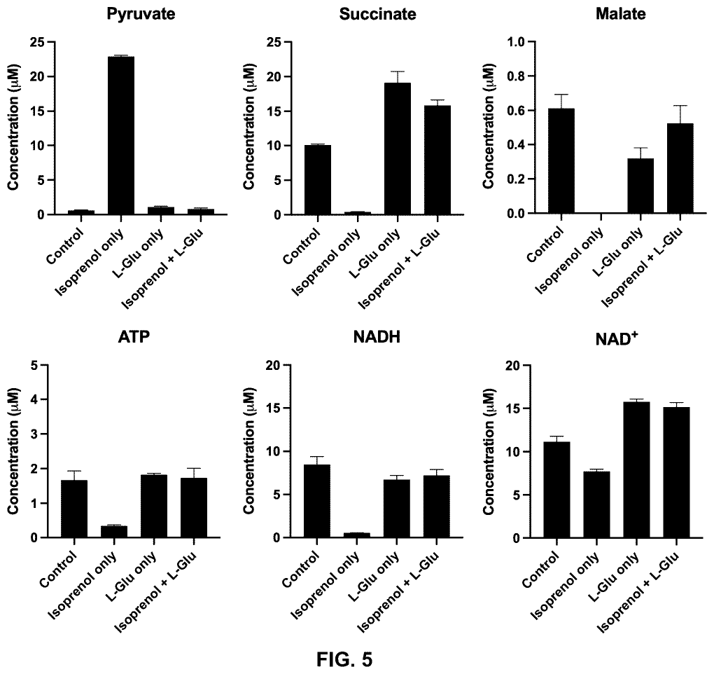

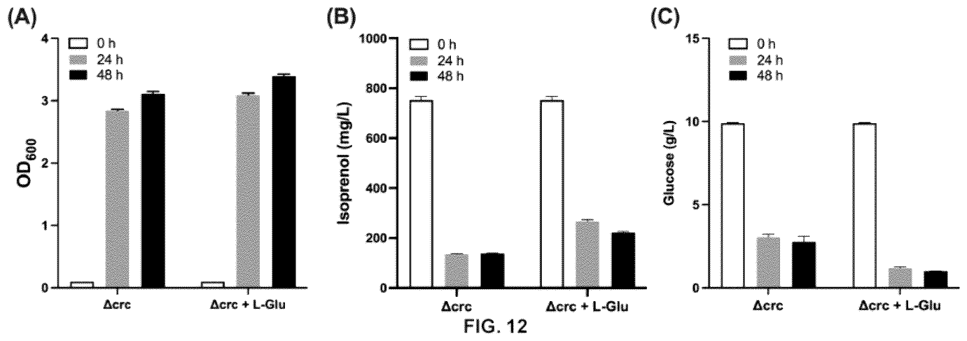

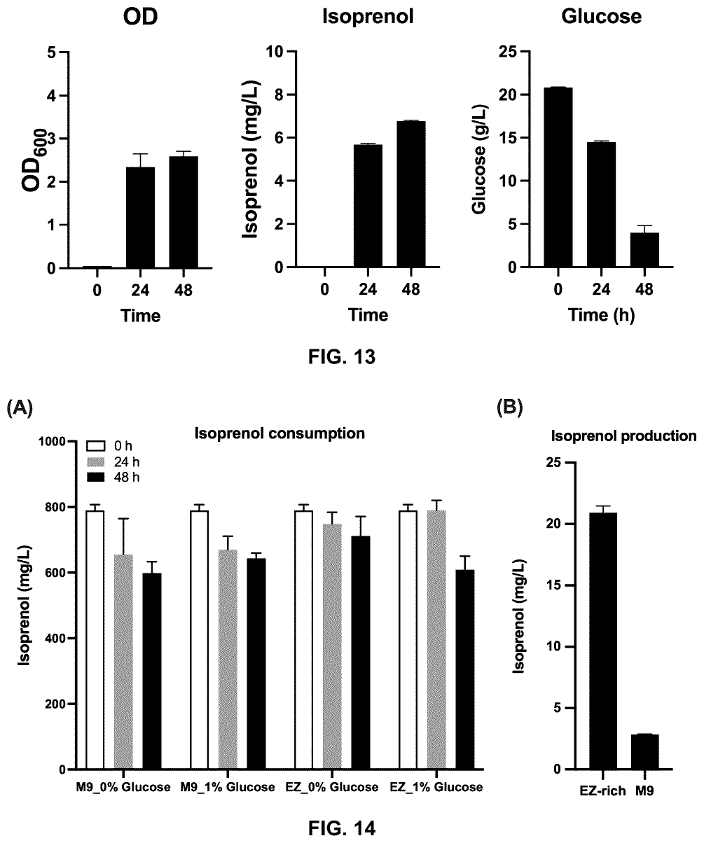

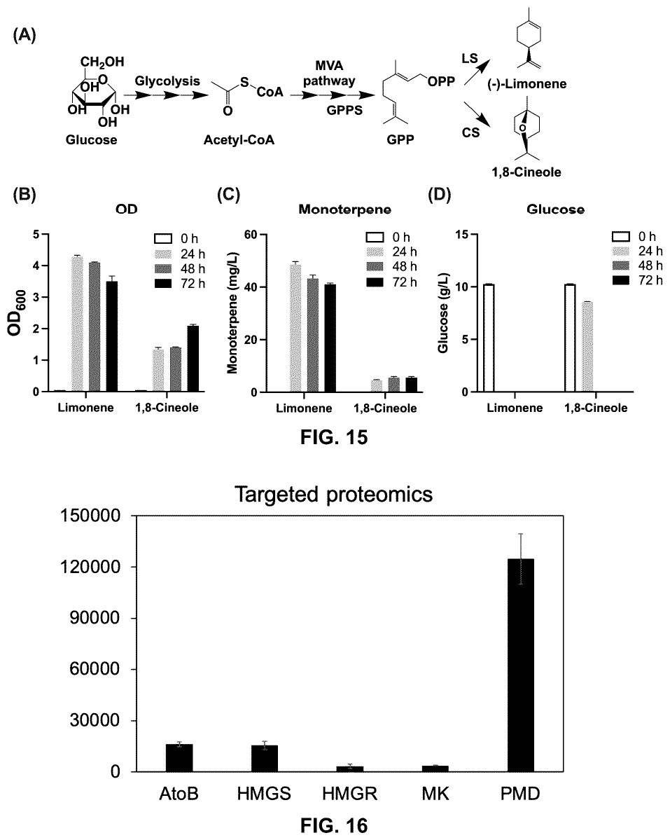

Based on the findings of the L-Glu supplementation experiment, we continued to investigate the mechanism that L-Glu involves in isoprenol preservation in P. putida. We compared the intracellular metabolites between the conditions with and without the L-Glu supplement. When isoprenol is presented in the medium without the L-Glu supplement, it showed a significant difference in metabolites of central carbon and energy metabolism after 24 hours (FIG. 5). Although isoprenol could provide an additional carbon source, the difference in pyruvate, succinate, malate, ATP, NADH, and NAD+ levels indicated an insufficient energy supply and imbalanced redox compared with the control group. In contrast, supplementing L-Glu restored those metabolites to comparable levels to the control group (FIG. 5). Since L-Glu is considered as a favored carbon source for P. putida [39], the L-Glu-mediated prevention of isoprenol self-consumption could be attributed to carbon catabolite repression (CCR). To verify this, we deleted the CCR regulator gene (crc) from the P. putida chromosome and studied the isoprenol consumption with the Acre strain (JPUB_019978, Table 1). Results showed the prevention of isoprenol from self-consumption by supplementing L-Glu was significantly reduced when crc is deleted (222 mg/L, FIG. 12, B), compared with the strain without crc deletion (523 mg/L, FIG. 4, B). This suggests L-Glu assisted isoprenol preservation in P. putida may be attributed to CCR, in which L-Glu is a preferred carbon source, rather than isoprenol, in supporting rapid cell growth [40]. However, overexpressing crc with the isoprenol pathway did not increase isoprenol production but even lower the production titer (6 mg/L, FIG. 13).

3.4 Isoprenol Production Using p-Coumarate as a Carbon Source.

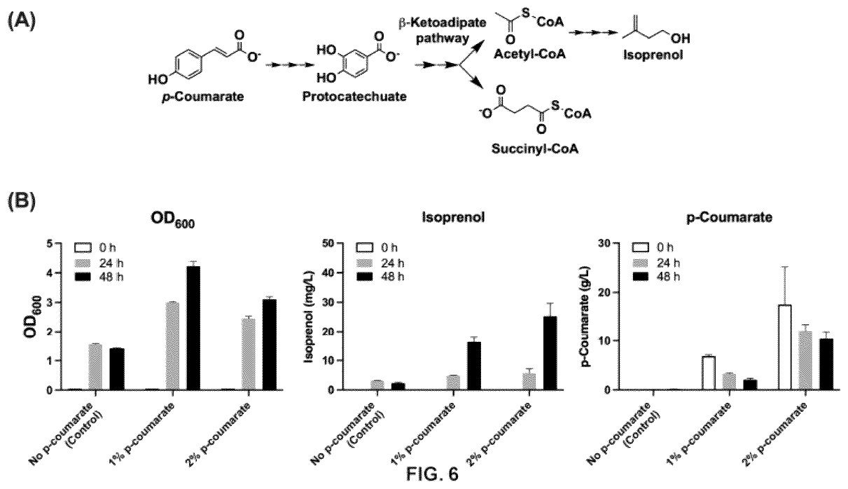

p-Coumarate is a prominent compound used as a representative lignin derived aromatics and there are efforts to increase p-coumarate content in lignocellulosic biomass (Tian et al., 2021). We attempted to use p-coumarate as the carbon source to investigate isoprenol production in the engineered P. putida strain. Results showed that the engineered P. putida strain (JPUB_019977, Table 1) can produce up to 25 mg/L isoprenol from 2% p-coumarate (c.f. the maximum theoretical yield from p-coumarate is 0.273 g/g p-coumarate) after 48 hours (FIG. 6), which is 24% of the isoprenol titer achieved from 2% glucose. We observed that the cell growth at 2% p-coumarate was 27% lower than the 1% p-coumarate condition after 48 hours. More residual p-coumarate was detected in the medium for the 2% condition (FIG. 6), suggesting a higher concentration of p-coumarate may inhibit cell growth. Though the isoprenol titer was lower from p-coumarate than from glucose, it showed the possibility of utilizing aromatics as well as sugars as the carbon source in biofuel production. This demonstrates that P. putida is a promising host for the comprehensive conversion of carbons from lignocellulosic biomass for bio-based production.

3.5 Engineering P. putida for Other Larger Terpenes Production.

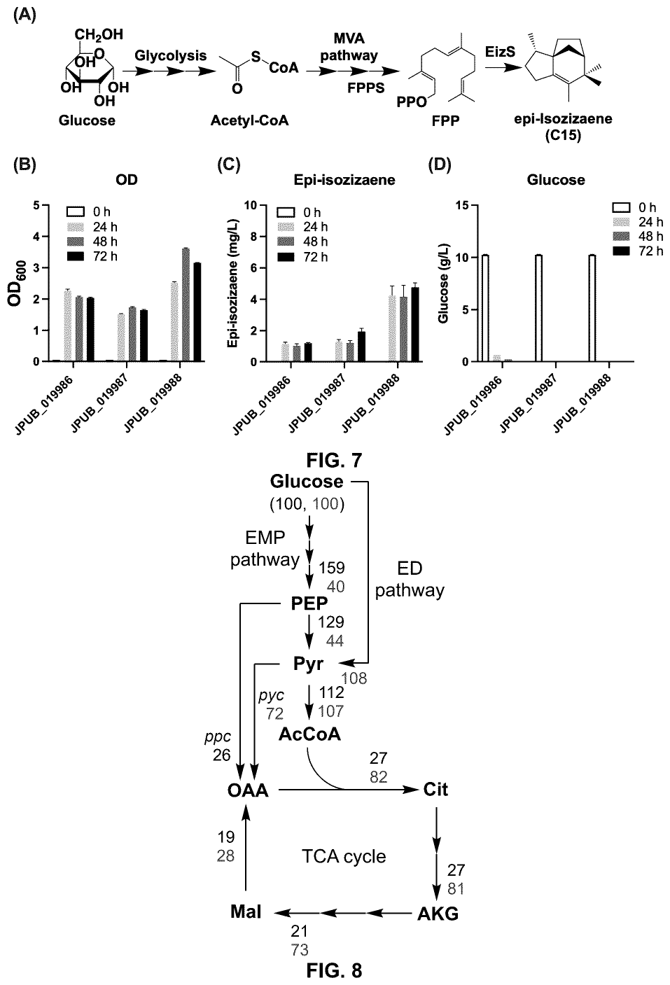

To expand the isoprenoid production profile in P. putida via the MVA pathway, we engineered the MVA pathway for monoterpenes and sesquiterpenes. We choose two monoterpenes (limonene and 1,8-cincole) and one sesquiterpene (epi-isozizaene) as targets for production. The MEP pathway was used as a control by overexpressing the epi-isozizaene synthase. As shown in FIG. 7, the MVA pathway (2 mg/L) showed a higher level of epi-isozizaene than the MEP pathway (1 mg/L). By applying the ΔphaABC strain for the MVA pathway, a higher production for epi-isozizaene (5 mg/L) was observed from 1% glucose, which is consistent with the results obtained from isoprenol production. However, compared with isoprenol, the production level of epi-isozizaene was much lower, and more efforts are needed to optimize the pathway for sesquiterpenes.

### 4. DISCUSSION

In this study, we engineered the heterologous MVA pathway in P. putida KT2440 to produce isoprenoids, including isoprenol (C₅) and epi-isozizaene (C₁₅). Unlike the E. coli system, the use of a heterologous MVA pathway showed very limited improvement of isoprenoid production (FIGS. 2 and 7). These results are consistent with a previous report and the reason might be the result of the distinct central metabolism in P. putida and its different flux distribution with acetyl-CoA (FIG. 8). For isoprenol, we also engineered the IPP-bypass MVA pathway, and it showed advantages compared with using the MEP and the original MVA pathway. The highest isoprenol titer from engineered P. putida was 104 mg/L from 2% glucose.

While the use of the IPP-bypass MVA pathway made a substantial improvement during isoprenol production, this is still much lower than the batch culture titer (˜2,500 mg/L) reported in E. coli [24]. Compared with the E. coli system, the low isoprenol titer might be attributed to two reasons. First, the isoprenol degradation pathway in P. putida competes with the synthesis pathway, leading to a reduced accumulation of isoprenol. In contrast, E. coli does not show the capability of consuming isoprenol as a carbon source. Due to isoprenol consumption being very significant in P. putida (up to 714 mg/L isoprenol was consumed in 24 hours, FIG. 4, A), this could be one of the main reasons that the engineered P. putida cannot show a comparable isoprenol titer to the similarly engineered E. coli strains. We tried deleting a gene (PP_2675) reported to be associated with P. putida's growth on isoprenol [28], but this deletion did not improve the isoprenol titer (FIG. 14). As many genes have been identified as being involved in isoprenol catabolism [28], additional gene deletion may be required to achieve reduction in isoprenol catabolism and degradation without compromising isoprenol production. Second, the balancing of isoprenol-pathway enzymes was harder to achieve in P. putida than in E. coli as the number and the variety of plasmids are limited in P. putida. In the E. coli system, two plasmids could be used for isoprenol production to achieve a well-balanced pathway proteins expression for the best isoprenol production. For example, the 1^{st} plasmid was selected to be a medium to low copy plasmid to drive the top MVA portion genes (atoB, HMGS, HMGR) to reduce accumulation of the final metabolite (mevalonate) and prevent the substrate inhibition against the next enzyme (MK) in the pathway. The 2^{nd} plasmid contained a high copy origin and drove MK and PMD genes expression under a strong promoter as higher level expression of these enzyme are required to drive the pathway toward the product [24]. Therefore to optimize the balancing of pathway enzymes in P. putida, a systematic comparison of pathway constructs such as using a 1-plasmid vs 2-plasmid system, varying plasmid copy numbers, and changing the strength of the promoter or RBS in driving different pathway genes will be required as previously shown in the E. coli isoprenoid studies [41].

As P. putida consumes isoprenol, we investigated the possibilities of preventing the consumption of isoprenol by supplementing specific medium components. Interestingly, we found supplementing L-Glu in the culture medium showed a significant preservation of isoprenol. Using metabolomics, we revealed the difference of intracellular metabolites and attempted to explain the possible scenarios during isoprenol degradation. The metabolites analysis showed an insufficient energy availability and an imbalanced redox status during isoprenol degradation. This may be associated with the alcohol degradation mechanism as P. putida utilizes pyrroloquinoline quinone (PQQ)-dependent alcohol dehydrogenases for alcohol degradation (Matthias et al., 2022), which may change the balance of cellular redox when processing isoprenol degradation. In addition, since L-Glu is a precursor of PQQ biosynthesis [42], supplementing L-Glu could increase substrate availability toward PQQ biosynthesis, which might contribute to the rebalancing of redox status as well as restoring the cellular metabolism. On the other hand, a few studies reported the development of isoprenol utilization pathways for isoprenoid synthesis, such as isopentenol utilization pathway (IUP) and isoprenoid alcohol (IPA) pathway [44]. In these pathways, the alcohol kinase (e.g. yeast choline kinase, [43]) was identified and used for isoprenol phosphorylation, which indicated alcohol phosphorylation might be alternative route beside alcohol dehydrogenation related to isoprenol degradation.

As P. putida is an emerging microbial host, there are still many challenges to engineer this host as a bioproduction workhorse. For example, even though some P. putida species can utilize xylose as a carbon source, the most widely studied P. putida microbial platform (KT2440) cannot naturally utilize xylose. Thus, engineering for the simultaneous utilization of glucose, xylose, and lignin-derived aromatic substrates may need additional efforts to achieve optimal carbon utilization without comprising the production yields [41,42]. The versatile metabolism of P. putida which allows it to survive with broad substrates also brings issues of the self-degradation of biosynthetic products. These issues could be challenging to overcome since multiple genes and regulations may be involved in the degradation process [28]. Additionally, the polyploid property nature of P. putida may increase the instability of using a high-copy plasmid for gene expression [45], and consistent with this we observed significant variations among colonies when screening for productions. Even with these issues, the unique capability of P. putida to utilize lignin-derived intermediates and aromatics as carbon sources are clear advantages over the widely used microbial hosts such as E. coli and S. cerevisiae as a next-generation industrial microbial host for converting lignocellulosic biomass to biofuels and bioproducts. In this study, we demonstrated that the engineered P. putida strains can utilize p-coumarate, as the sole carbon source to produce isoprenol. It is foreseeable that P. putida can achieve an economically feasible production of isoprenol and other bio-based products from lignocellulosic biomass via systematic strain engineering combining the efforts of computation and analytics using the Design-Build-Test-Learn research cycle [46].

### 5. CONCLUSIONS

P. putida can naturally utilize broad carbon sources and is tolerant to xenobiotics, which shows great potential to be developed as an emerging industrial microbial workhorse especially in maximally converting carbon from lignocellulosic biomass to biofuels and bioproducts. In this study, we engineered the heterologous MVA pathway in P. putida KT2440 to produce isoprenoids, including isoprenol (C₅) and epi-isozizaene (C₁₅). IPP-bypass MVA pathway showed advantages during isoprenol production. Through comparing flux distribution and identifying gene knockout target, we optimized the production strain to achieve an increase of isoprenol production to 104 mg/L in a batch flask experiment. Due to the isoprenol degradation in P. putida, we investigated the strategy to prevent self-consumption of isoprenol, and supplementation of L-Glu in the medium was found to show significant preservation for isoprenol. The engineered P. putida strain can also produce isoprenol using p-coumarate as the sole carbon source. Our results presented a good demonstration of developing P. putida as a new microbial chassis for biofuel production with improved carbon utilization from lignocellulosic biomass.

References cited in Example 1 and the “BACKGROUND OF THE INVENTION” section:
1. Keasling J, Garcia Martin H, Lee T S, Mukhopadhyay A, Singer S W, Sundstrom E. Microbial production of advanced biofuels. Nature Reviews Microbiology. 2021; 19:701-15
2. Liu Y, Cruz-Morales P, Zargar A, Belcher M S, Pang B, Englund E, et al. Biofuels for a sustainable future. Cell. Elsevier; 2021; 184:1636-47.
3. Rubin E M. Genomics of cellulosic biofuels. Nature. 2008; 454:841-5.
4. Kamimura N, Takahashi K, Mori K, Araki T, Fujita M, Higuchi Y, et al. Bacterial catabolism of lignin-derived aromatics: New findings in a recent decade: Update on bacterial lignin catabolismenvironmental Microbiology Reports. John Wiley & Sons, Ltd; 2017; 9:679-705.
5. Xu Z, Lei P, Zhai R, Wen Z, Jin M. Recent advances in lignin valorization with bacterial cultures: microorganisms, metabolic pathways, and bio-products. Biotechnology for Biofuels. 2019; 12:32.
6. Becker J, Wittmann C. A field of dreams: Lignin valorization into chemicals, materials, fuels, and health-care products. Biotechnology Advances. 2019; 37:107360.
7. Baral N R, Yang M, Harvey B G, Simmons B A, Mukhopadhyay A, Lee T S, et al. Production Cost and Carbon Footprint of Biomass-Derived Dimethylcyclooctane as a High-Performance Jet Fuel Blendstock. ACS Sustainable Chem Eng. American Chemical Society; 2021; 9:11872-82.
8. Weimer A, Kohlstedt M, Volke D C, Nikel P I, Wittmann C. Industrial biotechnology of Pseudomonas putida: advances and prospects. Applied Microbiology and Biotechnology. 2020; 104:7745-66.
9. Nikel P I, Chavarría M, Danchin A, de Lorenzo V. From dirt to industrial applications: Pseudomonas putida as a Synthetic Biology chassis for hosting harsh biochemical reactions. Current Opinion in Chemical Biology. 2016; 34:20-9.
10. Weinel C, Nelson K E, Tümmler B. Global features of the Pseudomonas putida KT2440 genome sequence. Environmental Microbiology. John Wiley & Sons, Ltd; 2002; 4:809-18.
11. Nikel P I, Chavarría M, Fuhrer T, Sauer U, de Lorenzo V. Pseudomonas putida KT2440 Strain Metabolizes Glucose through a Cycle Formed by Enzymes of the Entner-Doudoroff, Embden-Meyerhof-Parnas, and Pentose Phosphate Pathways. Journal of Biological Chemistry. 2015; 290:25920-32.
12. Peralta-Yahya P P, Zhang F, del Cardayre S B, Keasling J D. Microbial engineering for the production of advanced biofuels. Nature. 2012; 488:320-8.
13. Li M, Hou F, Wu T, Jiang X, Li F, Liu H, et al. Recent advances of metabolic engineering strategies in natural isoprenoid production using cell factories. Natural Product Reports. The Royal Society of Chemistry; 2020; 37:80-99.
14. George K W, Thompson M G, Kang A, Baidoo E, Wang G, Chan L J G, et al. Metabolic engineering for the high-yield production of isoprenoid-based C5 alcohols in E. coli. Scientific Reports. 2015; 5:11128.
15. Alonso-Gutierrez J, Chan R, Batth T S, Adams P D, Keasling J D, Petzold C J, et al. Metabolic engineering of Escherichia coli for limonene and perillyl alcohol production. Metabolic Engineering. 2013; 19:33-41.
16. Mendez-Perez D, Alonso-Gutierrez J, Hu Q, Molinas M, Baidoo E E K, Wang G, et al. Production of jet fuel precursor monoterpenoids from engineered Escherichia coli. Biotechnology and Bioengineering. John Wiley & Sons, Ltd; 2017; 114:1703-12.
17. Peralta-Yahya P P, Ouellet M, Chan R, Mukhopadhyay A, Keasling J D, Lee T S. Identification and microbial production of a terpene-based advanced biofuel. Nature Communications. 2011; 2:483.
18. Liu C-L, Tian T, Alonso-Gutierrez J, Garabedian B, Wang S, Baidoo E E K, et al. Renewable production of high density jet fuel precursor sesquiterpenes from Escherichia coli. Biotechnology for Biofuels. 2018; 11:285.
19. Gupta P, Phulara S C. Metabolic engineering for isoprenoid-based biofuel production. Journal of Applied Microbiology. John Wiley & Sons, Ltd; 2015; 119:605-19.
20. Kim J, Baidoo E E K, Amer B, Mukhopadhyay A, Adams P D, Simmons B A, et al. Engineering Saccharomyces cerevisiae for isoprenol production. Metabolic Engineering. 2021; 64:154-66.
21. Rosenkoetter K E, Kennedy C R, Chirik P J, Harvey B G. [4+4]-cycloaddition of isoprene for the production of high-performance bio-based jet fuel. Green Chemistry. The Royal Society of Chemistry; 2019; 21:5616-23.
22. Kang A, George K W, Wang G, Baidoo E, Keasling J D, Lee T S. Isopentenyl diphosphate (IPP)-bypass mevalonate pathways for isopentenol production. Metabolic Engineering. 2016; 34:25-35.
23. George K W, Thompson M G, Kim J, Baidoo E E K, Wang G, Benites V T, et al. Integrated analysis of isopentenyl pyrophosphate (IPP) toxicity in isoprenoid-producing Escherichia coli. Metabolic Engineering. 2018; 47:60-72.
24. Kang A, Mendez-Perez D, Goh E-B, Baidoo E E K, Benites V T, Beller H R, et al. Optimization of the IPP-bypass mevalonate pathway and fed-batch fermentation for the production of isoprenol in Escherichia coli. Metabolic Engineering. 2019; 56:85-96.
25. Mi J, Becher D, Lubuta P, Dany S, Tusch K, Schewe H, et al. De novo production of the monoterpenoid geranic acid by metabolically engineered Pseudomonas putida. Microbial Cell Factories. 2014; 13:170.
26. Hernandez-Arranz S, Perez-Gil J, Marshall-Sabey D, Rodriguez-Concepcion M. Engineering Pseudomonas putida for isoprenoid production by manipulating endogenous and shunt pathways supplying precursors. Microbial Cell Factories. 2019; 18:152.
27. Yang J, Son J H, Kim H, Cho S, Na J, Yeon Y J, et al. Mevalonate production from ethanol by direct conversion through acetyl-CoA using recombinant Pseudomonas putida, a novel biocatalyst for terpenoid production. Microbial Cell Factories. 2019; 18:168.
28. Thompson M, Incha M, Pearson A, Schmidt M, Sharpless W, Christopher E, et al. Fatty Acid and Alcohol Metabolism in Pseudomonas putida: Functional Analysis Using Random Barcode Transposon Sequencing. Applied and Environmental Microbiology. American Society for Microbiology; 2020; 86: c01665-20.
29. Banerjee D, Eng T, Lau A K, Sasaki Y, Wang B, Chen Y, et al. Genome-scale metabolic rewiring improves titers rates and yields of the non-native product indigoidine at scale. Nature Communications. 2020; 11:5385.
30. Petra S, Juliane W, Hermann S, Lothar E. Identification of glyA (Encoding Serine Hydroxymethyltransferase) and Its Use Together with the Exporter ThrE To Increase 1-Threonine Accumulation by Corynebacterium glutamicum. Applied and Environmental Microbiology. American Society for Microbiology; 2002; 68:3321-7.
31. Wang X, Pereira J H, Tsutakawa S, Fang X, Adams P D, Mukhopadhyay A, et al. Efficient production of oxidized terpenoids via engineering fusion proteins of terpene synthase and cytochrome P450. Metabolic Engineering. 2021; 64:41-51.
32. Wang X, Goh E-B, Beller H R. Engineering E. coli for simultaneous glucose-xylose utilization during methyl ketone production. Microbial Cell Factories. 2018; 17:12.
33. Baidoo E E K, Wang G, Joshua C J, Benites V T, Keasling J D. Liquid Chromatography and Mass Spectrometry Analysis of Isoprenoid Intermediates in Escherichia coli BT-Microbial Metabolomics: Methods and Protocols. In: Baidoo EEK, editor. New York, NY: Springer New York; 2019; 209-24.
34. Lec T S, Krupa R A, Zhang F, Hajimorad M, Holtz W J, Prasad N, et al. BglBrick vectors and datasheets: A synthetic biology platform for gene expression. Journal of Biological Engineering. 2011; 5:12.
35. Gonzalez J E, Long C P, Antoniewicz M R. Comprehensive analysis of glucose and xylose metabolism in Escherichia coli under aerobic and anaerobic conditions by 13C metabolic flux analysis. Metabolic Engineering. 2017; 39:9-18.
36. Wang Q, Nomura C T. Monitoring differences in gene expression levels and polyhydroxyalkanoate (PHA) production in Pseudomonas putida KT2440 grown on different carbon sources. Journal of Bioscience and Bioengineering. 2010; 110:653-9.
37. Kang A, Meadows C W, Canu N, Keasling J D, Lee T S. High-throughput enzyme screening platform for the IPP-bypass mevalonate pathway for isopentenol production. Metabolic Engineering. 2017; 41:125-34.
38. Dugar D, Stephanopoulos G. Relative potential of biosynthetic pathways for biofuels and bio-based products. Nature Biotechnology. 2011; 29:1074-8.
39. Rojo F. Carbon catabolite repression in Pseudomonas: optimizing metabolic versatility and interactions with the environment. FEMS Microbiology Reviews. 2010; 34:658-84.
40. Molina L, Rosa R La, Nogales J, Rojo F. Pseudomonas putida KT2440 metabolism undergoes sequential modifications during exponential growth in a complete medium as compounds are gradually consumed. Environmental Microbiology. John Wiley & Sons, Ltd; 2019; 21:2375-90.
41. Alonso-Gutierrez J, Kim E-M, Batth T S, Cho N, Hu Q, Chan L J G, et al. Principal component analysis of proteomics (PCAP) as a tool to direct metabolic engineering. Metabolic Engineering. 2015; 28:123-33.
42. Puchringer S, Metlitzky M, Schwarzenbacher R. The pyrroloquinoline quinone biosynthesis pathway revisited: A structural approach. BMC Biochemistry. 2008; 9:8.
43. Chatzivasileiou A O, Ward V, Edgar S M, Stephanopoulos G. Two-step pathway for isoprenoid synthesis. Proceedings of the National Academy of Sciences. Proceedings of the National Academy of Sciences; 2019; 116:506-11.
44. Clomburg J M, Qian S, Tan Z, Cheong S, Gonzalez R. The isoprenoid alcohol pathway, a synthetic route for isoprenoid biosynthesis. Proceedings of the National Academy of Sciences. Proceedings of the National Academy of Sciences; 2019; 116:12810-5.
45. Cook T B, Rand J M, Nurani W, Courtney D K, Liu S A, Pfleger B F. Genetic tools for reliable gene expression and recombineering in Pseudomonas putida. Journal of Industrial Microbiology and Biotechnology. 2018; 45:517-27.
46. Carbonell P, Jervis A J, Robinson C J, Yan C, Dunstan M, Swainston N, et al. An automated Design-Build-Test-Learn pipeline for enhanced microbial production of fine chemicals. Communications Biology. 2018; 1:66.

### Example 2

### Genome-Scale and Pathway Engineering for the Sustainable Aviation Fuel Precursor Isoprenol Production in Pseudomonas putida

Sustainable aviation fuel (SAF) will significantly impact global warming in the aviation sector, and important SAF targets are emerging. Isoprenol is a precursor for a promising SAF compound DMCO (1,4-dimethylcyclooctane) and has been produced in several engineered microorganisms. Recently, Pseudomonas putida has gained interest as a future host for isoprenol bioproduction as it can utilize carbon sources from inexpensive plant biomass. Here, we engineer metabolically versatile host P. putida for isoprenol production. We employ two computational modeling approaches (Bilevel optimization and Constrained Minimal Cut Sets) to predict gene knockout targets and optimize the “IPP-bypass” pathway in P. putida to maximize isoprenol production. Altogether, the highest isoprenol production titer from P. putida was achieved at 3.5 g/L under fed-batch conditions. This combination of computational modeling and strain engineering on P. putida for an advanced biofuels production has vital significance in enabling a bioproduction process that can use renewable carbon streams.

Biological production of aviation fuels and their precursors from sustainable carbon sources stands to have a realistic impact on reducing CO₂ emissions, an increasingly critical aspect of addressing climate change1,2. For this reason, several sustainable aviation fuel (SAF) targets and their precursors are being proposed, which include not only traditional ethanol-based fuels3, but also novel high-energy multicyclic compounds possible via bioproduction, such as fuelimycin A4 and epi-isozizaene5,6. One such important SAF precursor is isoprenol (a.k.a 3-methylbut-3-en-1-ol). Isoprenol is a commodity platform chemical and a vetted biogasoline7, and it is also the precursor to the jet fuel 1,4-dimethyl cyclooctane (DMCO). Catalytic conversion of isoprenol to DMCO has been shown at high efficiency8 and establishing a carbon-efficient conversion of renewable carbon sources to isoprenol would enable a highly sustainable process8 for DMCO.

While isoprenol production has been shown in model microbial hosts (Escherichia coli9, Corynebacterium glutamicum10, and Saccharomyces cerevisiae11), catabolically versatile microbes that consume a wider range of carbon compounds are essential to providing a cost-effective process1,12. In the case of plant biomass conversion, there is an urgent need to demonstrate production of isoprenol in microbial systems that can catabolize both sugars and aromatics derived from lignocellulosic biomass. Pseudomonas putida KT2440 is an ideal conversion host with a versatile conversion profile13,14 and efficient genetic tools. While prior works in model organisms15,16 have achieved robust isoprenol titers, a microbial host such as P. putida KT2440 is a more likely candidate for the final deployment for isoprenol production as a SAF precursor. A less-commonly used laboratory host such as P. putida, however, is a far more challenging system to develop as a conversion platform. For instance, the most efficient route to isoprenol is through the heterologous mevalonate (MVA) pathway using an IPP-bypass that utilizes hydroxymethylglutaryl CoA (HMG-COA) as the precursor 15. However, efforts in P. putida17 have shown that the mere MVA pathway overexpression did not provide any improvements over the native 2-C-methyl-D-erythritol 4-phosphate (MEP) pathway overexpression and both resulted in very low titers. A similar observation was also reported in cyanobacteria 18.

In a recent study, we were able to establish the MVA pathway in P. putida KT244019 and define the necessary cultivation conditions to produce isoprenol in this host via the heterologous pathway. While this provides an excellent foundation for isoprenol production in P. putida KT2440, this microbes' unusual metabolic profile presents several challenges that need to be overcome. One issue is the catabolism of isoprenol itself, and its intermediates, by P. putida KT2440. Extensive functional genomics data have recently been accumulated for P. putida KT2440 and have revealed genes associated with degradation or catabolism of non-canonical carbon sources (e.g., levulinic acid20, lysine21, and isoprenol22), and also provided the hypotheses for host engineering targets to optimize the desired catabolism and minimize the undesired ones.

Computationally driven metabolic engineering methods have gained interest during the last decade23. Such methods can predict strategies that may involve large numbers of genetic interventions (e.g. deletion, overexpression, or repression) to reach the predicted yields. Implementation of such strategies is sometimes challenging even with recent advances in synthetic biology and metabolic engineering techniques. To address this challenge and cover a larger solution space we used multiple computational strain design methods based on elementary mode analysis or bilevel optimization. The latest highly curated genome-scale metabolic model (GSMM) for P. putida24 enabled the use of these approaches and also highlighted the differences in metabolism from model microbes such as E. coli.

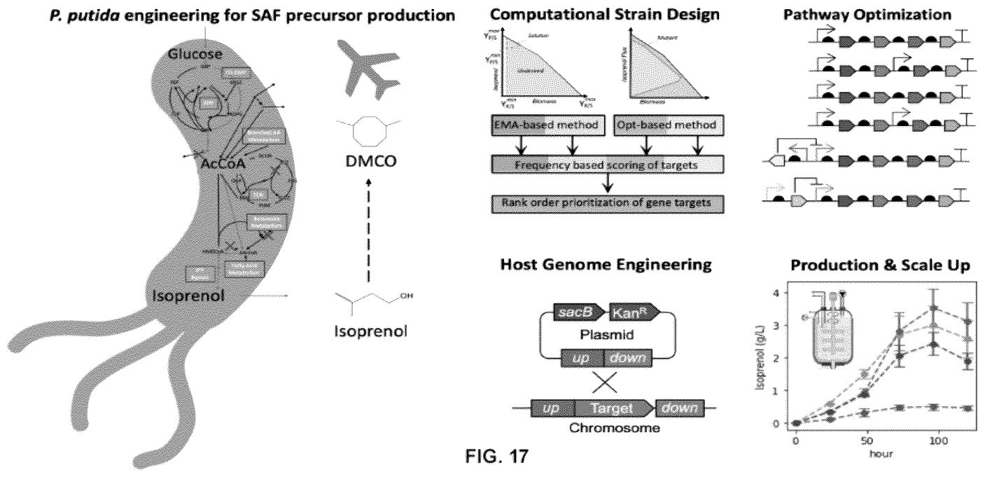

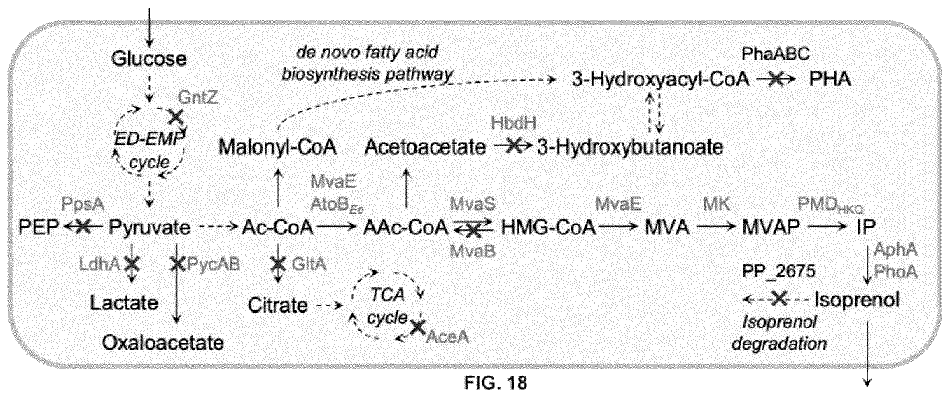

In this work, we employ two GSMM-guided approaches in combination with targeted edits and pathway improvements to enhance the production of the DMCO precursor, isoprenol, in P. putida KT2440 (FIG. 17). We first add the knowledge from functional genomics data sets (e.g., genes involved in isoprenol degradation) and the heterologous MVA pathway to update the GSMM (FIG. 18). We then use both Elementary mode analysis (EMA)-based methods25,26 and bilevel optimization (Opt)-based methods27,28 to prioritize a subset of host genome targets that, when deleted, are predicted to enhance flux to isoprenol via the MVA route. We also use known edits such as deletion of the pha operon and other literature-based targets to further enhance isoprenol titers. Finally, we use proteomics to optimize the pathway configuration. Overall, our GSMM-guided approach allowed us to select and prioritize the intervention targets, and lead to over 3.5 g/L isoprenol from glucose in a minimal defined medium under fed-batch conditions. This titer is the highest reported for P. putida KT2440 and has vital significance in enabling a bioproduction process that can use renewable carbon streams as the starting material.

### RESULTS AND DISCUSSION

### Computational Strain Design for Isoprenol Production

For model-guided improvement of isoprenol production, we employed EMA-based approaches including Elementary Flux Modes (EFMs) 25 and Constrained Minimal Cut Sets (cMCS) 26 as well as Opt-based approaches including OptKnock27,29 and OptForce28 using the latest GSMM for P. putida iJN146224 augmented with the heterologous MVA pathway (Supplementary File 1) and a central metabolic model for P. putida with a lumped reaction for the heterologous MVA pathway (Supplementary File 2). Preliminary computational strain design results showed that growth-coupled production of isoprenol requires the deletion of 9 or more metabolic reactions in P. putida. Construction of such mutants would require significant experimental efforts and testing of intermediate mutants to monitor the progress. However, it is not clear from computational predictions which genes are more important for increasing isoprenol production and therefore should be knocked out with high priority since the growth-coupled production does not happen in silico with the deletion of a subset of identified reactions. To this end, we generated a large number of computational designs using EMA-based and Opt-based methods and calculated the frequency of knockout targets appearing in the designs by each method. The frequency was used to calculate the rank order of targets for each method, and the rank order from different methods was combined to calculate the final score. Our assumption was that certain targets can be more important for improving isoprenol production (e.g., due to higher fluxes or key branch points) than others and thus will appear more frequently in a diverse set of computational designs. By generating a large number of designs using multiple computational methods and combining them using a rank-based ensemble approach, we aimed to identify such crucial targets and prioritize them for the experimental construction of knockout strains. Although the computational model requires the deletion of all targets from a design to see improved isoprenol production, we hypothesized that the deletion of a subset consisting of these crucial targets will still lead to improved isoprenol production.

For the EMA-based methods, we first calculated EFMs using the central metabolic model. Each EFM is a minimal set of reactions carrying flux under the defined glucose minimal medium condition for growth as well as isoprenol production. A total of 360,475 EFMs were computed of which only 276 EFMs were selected that carried a flux through the biomass, ATP maintenance, glucose uptake, and isoprenol production reactions. A frequency-based scoring was used to prioritize targets, from 276 different computed EFMs (Supplementary File 3). Further we used cMCS to compute growth-coupled strategies for isoprenol production using the GSMM. From a total of 60 cMCS runs, we enumerated 4,950 feasible cMCS cut set designs. We used a frequency-based scoring to prioritize targets from the feasible cMCS designs that were computed for isoprenol and its precursors HMG-COA, DMAPP or IPP (Supplementary File 3).

For the Opt-based methods, OptKnock was first used to find knockouts to couple isoprenol production to growth using the GSMM. A total of 157 OptKnock solutions were initially collected and pre-processed to 120 solutions by removing redundant solutions. In addition, we constrained the model by blocking the secretion of byproducts except for experimentally observed ones (e.g., gluconate, 2-ketogluconate, and acetate) to find another set of designs. Using the constrained model, 377 OptKnock solutions were obtained and pre-processed to 263 solutions. OptForce was next used to identify strategies to improve isoprenol production using the GSMM. A total of 50 OptForce solutions were obtained, but we found that they consisted mostly of routes that increase or decrease flux and included only 9 knockout targets with low frequencies. Therefore, we decided to use the OptKnock solutions from two simulations to calculate the frequency for scoring gene targets (Supplementary File 4).

Finally, we combined scores from EMA-based and Opt-based predictions to arrive at the top 8 priority gene targets for experimental implementation (Table 4 and FIG. 18). The first two priority gene targets (mvaB and hbdH) were involved in the degradation of endogenous metabolites (HMG-COA and acetoacetyl-CoA) that also participate in the heterologous MVA pathway. The other priority gene targets were involved in central carbon metabolism including the pentose phosphate pathway (gntZ), pyruvate metabolism (ldhA, ppsA, and pycAB), and TCA cycle (gltA and aceA).

TABLE 4
Strains and plasmids used in this study

| | Description | Reference |
|---|---|---|
| Strains | | |
| JPUB_019964 | P. putida KT2440 deleted with the phaA-phaB-phaC gene | This study |
| (ΔphaABC) | cluster (PP_5003-PP_5005) | |
| JPUB_019965 | P. putida KT2440 ΔphaABC ΔPP_2675 | This study |
| JPUB_019966 | P. putida KT2440 with pBbB1k-NudB | This study |
| JPUB_019967 | P. putida KT2440 with pBbB5k-MTSA-T1-MK_{sc}-PMK- | This study |
| | PMD_{sc}-NudB | |
| JPUB_019968 | P. putida KT2440 with pBbB5k-AtoB-HMGS_{sc}-HMGR_{sc}- | This study |
| | T1-MK_{sc}-PMD_{sc} | |
| JPUB_019969 | P. putida KT2440 with pBbB5k-AtoB-HMGSₛₐ-HMGRₛₐ- | This study |
| | T1-MK_{sc}-PMD_{sc} | |
| JPUB_019971 | P. putida KT2440 with pBbB5k-MvaS_{ef}-MvaE_{ef}-T1-MK_{sc}- | This study |
| | PMD_{sc} | |
| JPUB_019973 | P. putida ΔphaABC with pBbB5k-AtoB-HMGS_{sc}-HMGR_{sc}- | This study |
| | T1-MK_{sc}-PMD_{sc} | |
| JPUB_019974 | P. putida ΔphaABC with pBbB5k-MvaS_{ef}-MvaE_{ef}-T1- | This study |
| | MK_{sc}-PMD_{sc} | |
| JPUB_019975 | P. putida ΔphaABC with pBbB5k-MvaS_{ef}-MvaE_{ef}-T1- | This study |
| | MK_{sc}-PMD_{HKQ} | |
| JPUB_019976 | P. putida ΔphaABC with pBbB5k-MvaS_{ef}-MvaE_{ef}-T1- | This study |
| | MKₘₘ-PMD_{sc} | |
| JPUB_019977 | P. putida ΔphaABC with pBbB5k-MvaS_{ef}-MvaE_{ef}-T1- | This study |
| | MKₘₘ-PMD_{HKQ} | |
| JPUB_019978 | P. putida ΔphaABC deleted with the crc gene (PP_5292) | This study |
| JPUB_019986 | P. putida KT2440 with pBbB1k-EizS | This study |
| JPUB_019987 | P. putida KT2440 with pBbB5k-MTSA-T1-MK_{sc}-PMK- | This study |
| | PMD_{sc}-idi-ispA-T1-EizS | |
| JPUB_019988 | P. putida ΔphaABC with pBbB5k-MTSA-T1-MK_{sc}-PMK- | This study |
| | PMD_{sc}-idi-ispA-T1-EizS | |
| Plasmids | | |
| JPUB_019914 | pBbB1k-NudB | This study |
| JPUB_019916 | pBbB5k-MTSA-T1-MK_{sc}-PMK-PMD_{sc}-NudB | This study |
| JPUB_019918 | pBbB5k-AtoB-HMGS_{sc}-HMGR_{sc}-T1-MK_{sc}-PMD_{sc} | This study |
| JPUB_019970 | pBbB5k-AtoB-HMGSₛₐ-HMGRₛₐ -T1-MK_{sc}-PMD_{sc} | This study |
| JPUB_019920 | pBbB5k-MvaS_{ef}-MvaE_{ef}-T1-MK_{sc}-PMD_{sc} | This study |
| JPUB_019922 | pBbB5k-MvaS_{ef-}MvaE_{ef} -T1-MK_{sc}-PMD_{HKQ} | This study |
| JPUB_019923 | pBbB5k-MvaS_{ef-}MvaE_{ef}-T1-MKₘₘ-PMD_{sc} | This study |
| JPUB_019925 | pBbB5k-MvaS_{ef-}MvaE_{ef}-T1-MKₘₘ-PMD_{HKQ} | This study |
| JPUB_019933 | pBbB1k-EizS | This study |
| JPUB_019935 | pBbB5k-MTSA-T1-MK_{sc}-PMK-PMD_{sc}-idi-ispA-T1-EizS | This study |
| JPUB_019939 | pK18-ppc | This study |
| JPUB_019941 | pK18-pyc | This study |
| JPUB_019943 | pK18-phaABC | This study |
| JPUB_019945 | pK18-crc | This study |
| JPUB_018413 | pNQ30-PP_2675 | [28] |
| JPUB_019949 | pBbB5k-MvaS_{ef-}MvaE_{ef}-T1-MKₘₘ-PMD_{HKQ}-crc | This study |

### Experimental Implementation of Metabolic Rewiring for Isoprenol Production

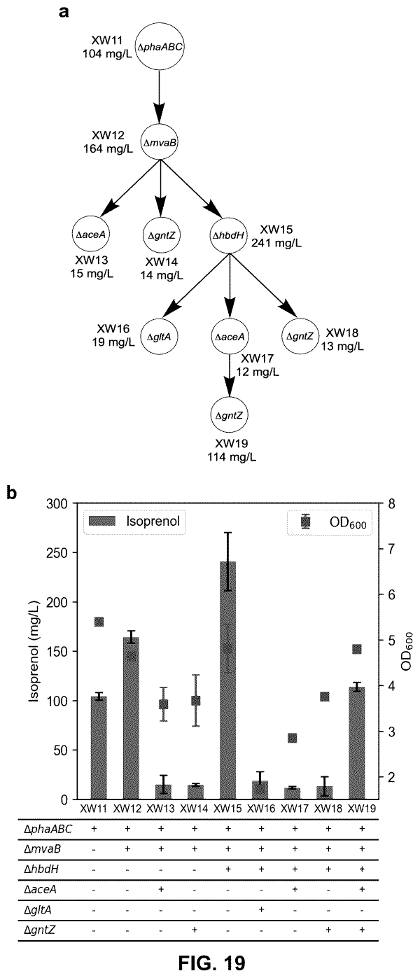

To experimentally verify the model-predicted targets. P. putida ΔphaABC strain (XW01, see Table 5 for the list of strains) was used as a background strain to perform gene knockouts. This strain has shown the highest isoprenol level in P. putida (104 mg/L, XW11 strain) when using the IPP-bypass MVA pathway via a plasmid (pXW1, see Supplementary Table 6 for the list of plasmids) in our previous work19. According to the model-predicted targets (Table 4), we constructed single and multiple gene knockout strains (FIG. 19, a). We started with the PP_3540/mvaB gene as it encodes for HMG-CoA lyase which catalyzes a reaction transforming HMG-COA into acetoacetate and acetyl-CoA, hence competing for HMG-CoA, a key precursor in mevalonate synthesis. The knockout of mvaB improved isoprenol production up to 164 mg/L, which increased 1.6-fold compared with the starter strain XW11 (FIG. 19, b).

TABLE 5
Strains used in this study

| Strains | JBEI Registry | Description | Reference |
|---|---|---|---|
| XW01 | JPUB_019964 | P. putida KT2440 ΔphaABC | Wang et al. 2022 |
| XW02 | JPUB_019990 | P. putida KT2440 ΔphaABC ΔmvaB | This study |
| XW03 | JPUB_019992 | P. putida KT2440 ΔphaABC ΔmvaB ΔaceA | This study |
| XW04 | JPUB_019994 | P. putida KT2440 ΔphaABC ΔmvaB ΔgntZ | This study |
| XW05 | JPUB_019996 | P. putida KT2440 ΔphaABC ΔmvaB ΔhbdH | This study |
| XW06 | JPUB_019998 | P. putida KT2440 ΔphaABC ΔmvaB ΔhbdH ΔgltA | This study |
| XW07 | JPUB_020000 | P. putida KT2440 ΔphaABC ΔmvaB ΔhbdH ΔaceA | This study |
| XW08 | JPUB_020002 | P. putida KT2440 ΔphaABC ΔmvaB ΔhbdH ΔgntZ | This study |
| XW09 | JPUB_020004 | P. putida KT2440 ΔphaABC ΔmvaB ΔhbdH ΔaceA ΔgntZ | This study |
| XW11 | JPUB_019977 | P. putida KT2440 ΔphaABC with plasmid pXW1 | Wang et al. 2022 |
| XW12 | JPUB_019991 | P. putida KT2440 ΔphaABC ΔmvaB with plasmid pXW1 | This study |
| XW13 | JPUB_019993 | P. putida KT2440 ΔphaABC ΔmvaB ΔaceA with plasmid pXW1 | This study |
| XW14 | JPUB_019995 | P. putida KT2440 ΔphaABC ΔmvaB ΔgntZ with plasmid pXW1 | This study |
| XW15 | JPUB_019997 | P. putida KT2440 ΔphaABC ΔmvaB ΔhbdH with plasmid pXW1 | This study |
| XW16 | JPUB_019999 | P. putida KT2440 ΔphaABC ΔmvaB ΔhbdH ΔgltA with plasmid | This study |
| | | pXW1 | |
| XW17 | JPUB_020001 | P. putida KT2440 ΔphaABC ΔmvaB ΔhbdH ΔaceA with plasmid | This study |
| | | pXW1 | |
| XW18 | JPUB_020003 | P. putida KT2440 ΔphaABC ΔmvaB ΔhbdH ΔgntZ with plasmid | This study |
| | | pXW1 | |
| XW19 | JPUB_020005 | P. putida KT2440 ΔphaABC ΔmvaB ΔhbdH ΔaceA ΔgntZ | This study |
| | | with plasmid pXW1 | |
| IY721 | JBEI-233609 | P. putida KT2440 ΔphaABC with plasmid pIY554 | This study |
| IY781 | JBEI-233601 | P. putida KT2440 ΔphaABC with plasmid pIY602 | This study |
| IY782 | JBEI-233603 | P. putida KT2440 ΔphaABC with plasmid pIY603 | This study |
| IY783 | JBEI-233605 | P. putida KT2440 ΔphaABC with plasmid pIY604 | This study |
| IY784 | JBEI-233607 | P. putida KT2440 ΔphaABC with plasmid pIY605 | This study |
| IY846 | JBEI-233611 | P. putida KT2440 ΔphaABC ΔmvaB ΔhbdH with plasmid | This study |
| | | pIY554 | |
| IY939 | JBEI-233615 | P. putida KT2440 ΔphaABC ΔmvaB ΔhbdH with plasmid | This study |
| | | pIY670 | |
| IY940 | JBEI-233617 | P. putida KT2440 ΔphaABC ΔmvaB ΔhbdH with plasmid | This study |
| | | pIY671 | |
| IY941 | JBEI-233619 | P. putida KT2440 ΔphaABC ΔmvaB ΔhbdH with plasmid | This study |
| | | pIY672 | |
| IY954 | JBEI-233613 | P. putida KT2440 ΔphaABC ΔmvaB ΔhbdH with plasmid | This study |
| | | pIY697 | |
| IY1049 | JBEI-233621 | P. putida KT2440 ΔphaABC ΔmvaB ΔhbdH with plasmid | This study |
| | | pIY761 | |
| IY1050 | JBEI-233623 | P. putida KT2440 ΔphaABC ΔmvaB ΔhbdH with plasmid | This study |
| | | pIY762 | |
| IY1054 | JBEI-233625 | P. putida KT2440 ΔphaABC ΔmvaB ΔhbdH with plasmid | This study |
| | | pIY765 | |
| IY1056 | JBEI-233627 | P. putida KT2440 ΔphaABC ΔmvaB ΔhbdH with plasmid | This study |
| | | pIY763 | |
| IY1101 | JBEI-233629 | P. putida KT2440 ΔphaABC ΔmvaB ΔhbdH ΔldhA | This study |
| | | with plasmid pIY672 | |
| IY1102 | JBEI-233631 | P. putida KT2440 ΔphaABC ΔmvaB ΔhbdH ΔppsA with plasmid | This study |
| | | pIY672 | |
| IY1200 | JBEI-233633 | P. putida KT2440 ΔphaABC ΔmvaB ΔhbdH ΔPP_2675 with | This study |
| | | plasmid pIY672 | |
| IY1245 | JBEI-233635 | P. putida KT2440 WT with plasmid pIY670 | This study |
| IY1246 | JBEI-233637 | P. putida KT2440 ΔPP_2675 with plasmid pIY670 | This study |
| IY1249 | JBEI-233639 | P. putida KT2440 ΔphaABC ΔmvaB with plasmid pIY670 | This study |
| IY1251 | JBEI-233641 | P. putida KT2440 ΔphaABC ΔmvaB ΔgntZ with plasmid pIY670 | This study |
| IY1252 | JBEI-233643 | P. putida KT2440 ΔphaABC ΔmvaB ΔhbdH ΔaceA | This study |
| | | with plasmid pIY670 | |
| IY1254 | JBEI-233645 | P. putida KT2440 ΔphaABC ΔmvaB ΔhbdH ΔaceA | This study |
| | | ΔgntZ with plasmid pIY670 | |
| IY1261 | JBEI-233647 | P. putida KT2440 ΔphaABC with plasmid pIY670 | This study |
| IY1262 | JBEI-233649 | P. putida KT2440 ΔphaABC ΔmvaB ΔhbdH ΔldhA | This study |
| | | with plasmid pIY670 | |
| IY1263 | JBEI-233651 | P. putida KT2440 ΔphaABC ΔmvaB ΔhbdH ΔppsA | This study |
| | | with plasmid pIY670 | |
| IY1319 | JBEI-233653 | P. putida KT2440 ΔmvaB with plasmid pIY670 | This study |
| IY1320 | JBEI-233655 | P. putida KT2440 AhbdH with plasmid pIY670 | This study |
| IY1452 | JBEI-233661 | P. putida KT2440 ΔphaABC ΔmvaB ΔhbdH ΔldhA | This study |
| | | ΔPP_2675 with plasmid pIY670 | |
| IY1884 | JBEI-233657 | P. putida KT2440 ΔphaABC ΔPP_2675 with | This study |
| | | plasmid pIY670 | |
| IY1885 | JBEI-233659 | P. putida KT2440 ΔphaABC ΔPP_2675 ΔldhA | This study |
| | | with plasmid pIY670 | |

TABLE 6
Plasmids used in this study.

| Plasmids | Description | Reference |
|---|---|---|
| pXW1 | pBbB5k-MvaS_{ef}-MvaE_{ef}-T1-MKₘₘ-PMD_{HKQ} | Wang et al. 2022 |
| pK18-mvaB | Plasmid to knockout mvaB (PP_3540) | This study |
| pK18-aceA | Plasmid to knockout aceA (PP_4116) | This study |
| pK18-gntZ | Plasmid to knockout gntZ (PP_4043) | This study |
| pK18-hbdH | Plasmid to knockout hbdH (PP_3073) | This study |
| pK18-gltA | Plasmid to knockout gltA (PP_4194) | This study |
| pK18-ldhA | Plasmid to knockout ldhA (PP_1649) | This study |
| pK18-ppsA | Plasmid to knockout ppsA (PP_2082) | This study |
| pIY554 | pRK2-Kan-araC-P_{BAD}-MvaS_{ef}-MvaE_{ef}-T_{rpoH}-P_{trc1-O}-MKₘₘ-PMD_{HKQ} | This study |
| pIY602 | pBBR1-B5-Kan-lacI-P_{lacUV5}-MvaS_{ef}-MvaE_{ef}-T_{rpoH}-P_{trc1-O}-MKₘₘ-PMD_{HKQ} | This study |
| pIY603 | pRK2-Kan-lacl-P_{lacUV5}-MvaS_{ef}-MvaE_{ef}-T_{rpoH}-P_{trc1-O}-MKₘₘ-PMD_{HKQ} | This study |
| pIY604 | pRSF1010-Kan-lacI-P_{lacUV5}-MvaS_{ef}-MvaE_{ef}-T_{rpoH}-P_{trc1-O}-MKₘₘ-PMD_{HKQ} | This study |
| pIY605 | pBBR1-Kan-lacI-P_{lacUV5}-MvaS_{ef}-MvaE_{ef}-T_{rpoH}-P_{trc1-O}-MKₘₘ-PMD_{HKQ} | This study |
| pIY670 | pRK2-Kan-araC-P_{BAD}-MvaS_{ef}-MvaE_{ef}-T_{rpoH}-P_{trc1-O}-MKₘₘ-PMD_{HKQ}-AphA | This study |
| pIY671 | pRK2-Kan-araC-P_{BAD}-MvaS_{ef}-MvaE_{ef}-T_{rpoH}-P_{trc1-O}-MKₘₘ-PMD_{HKQ}-NudB | This study |
| pIY672 | pRK2-Kan-araC-P_{BAD}-MvaS_{ef}-MvaE_{ef}-T_{rpoH}-P_{trc1-O}-MKₘₘ-PMD_{HKQ}-AphA- | This study |
| | NudB | |
| pIY697 | pRK2-Kan-araC-P_{BAD}-MvaS_{ef}-MvaE_{ef}-T_{rpoH}-P_{trc1-O}-MKₘₘ-PMD_{HKQ}-PhoA | This study |
| pIY761 | pRK2-Kan-araC-P_{BAD}-MvaS_{ef}-MvaE_{ef}-AtoB-T_{rpoH}-P_{trc1-O}-MKₘₘ-PMD_{HKQ}- | This study |
| | AphA-NudB | |
| pIY762 | pRK2-Kan-araC-P_{BAD}-MvaS_{ef}-MvaE_{ef}-NphT7-T_{rpoH}-P_{trc1-O}-MKₘₘ-PMD_{HKQ}- | This study |
| | AphA-NudB | |
| pIY763 | pRK2-Kan-araC-P_{BAD}-MvaS_{ef}-MvaE_{ef}-PMK-T_{rpoH}-P_{trc1-O}-MKₘₘ-PMD_{HKQ}- | This study |
| | AphA-NudB | |
| pIY765 | pRK2-Kan-araC-P_{BAD}-MvaS_{ef}-MvaE_{ef}-PMK-AtoB-T_{rpoH}-P_{trc1-O}-MKₘₘ- | This study |
| | PMD_{HKQ}-AphA-NudB | |
| pIY853 | pK18-PP_2675 | This study |

Using this double-knockout strain (ΔphaABC ΔmvaB) as a base, we performed a second round of gene knockouts with PP_4116/aceA (isocitrate lyasc), PP_4043/gntZ (6-phosphogluconate dehydrogenase), and PP_3073/hbdH (3-hydroxybutyrate dehydrogenase) (FIG. 19, a). While the knockout of aceA (XW13) or gntZ (XW14) significantly decreased isoprenol production to 14-15 mg/L, the hbdH knockout (XW15) improved isoprenol production up to 241 mg/L, a 2.3-fold increase from the XW11 strain and 1.5-fold increase from the XW12 strain, respectively (FIG. 19, b). As 3-hydroxybutyrate dehydrogenase (hbdH) catalyzes the conversion between 3-hydroxybutyrate and acetoacetate, the success of hbdH knockout in increasing isoprenol production may be attributed to the removal of a competing pathway of acetoacetyl-CoA, the first metabolite in the MVA pathway. Isocitrate lyase (aceA) converts isocitrate to glyoxylate, and 6-phosphogluconate dehydrogenase (gntZ) is a key enzyme at the shunt of the pentose phosphate pathway and the ED pathway. They are both involved in central carbon metabolism and the failure of these two knockouts in isoprenol improvement indicates that central carbon metabolism may require a significant rewiring by combinations of gene knockouts rather than a single gene deletion. Thus, engineering multiple gene knockouts in one strain may still be required to further improve isoprenol production.

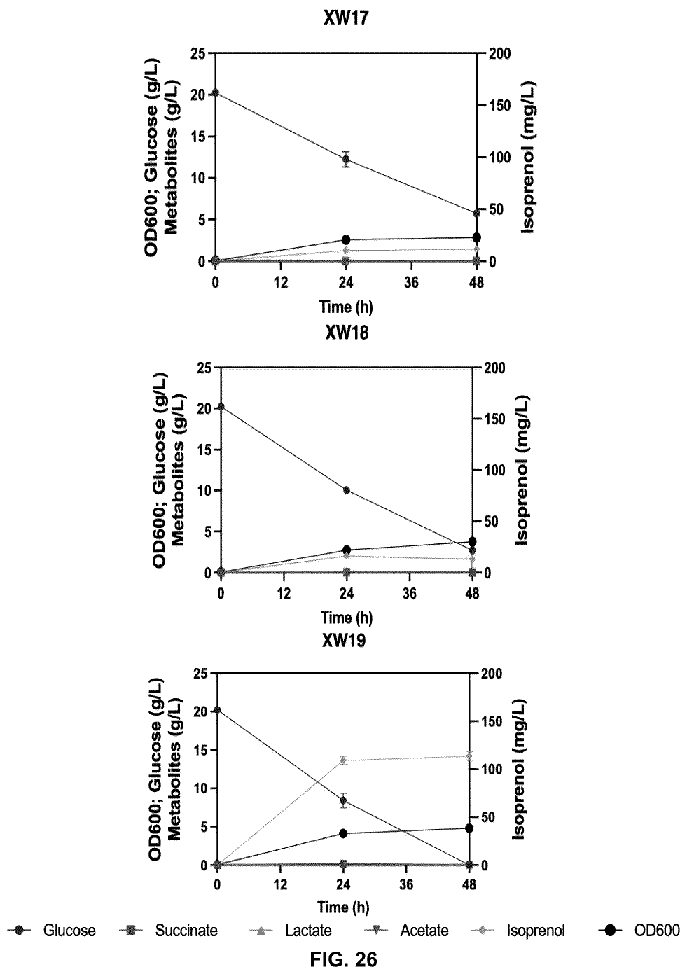

Therefore, for the third round of knockouts, we picked the highest producer with the triple knockouts (ΔphaABC ΔmvaB ΔhbdH, XW15) as a base, and performed the knockout of the genes involved in central carbon metabolism, gltA (citrate synthase), aceA (isocitrate lyasc), and gntZ (6-phosphogluconate dehydrogenase) (FIG. 19, a). Since citrate synthase is the first enzyme connecting glycolysis and the TCA cycle, it plays an important role in central carbon and energy metabolism. In P. putida KT2440, citrate synthase derives 3-fold more carbon flux from acetyl-CoA to TCA cycle compared with E. coli19. Thus, the knockout of gltA may limit the flux out of acetyl-CoA, which is desirable to support the MVA pathway flux and isoprenol production. However, experimental results (FIG. 19, b) showed that the deletion of gltA (XW16) significantly compromised cell growth and eventually lowered isoprenol production (19 mg/L). Given that the clear detriment of cell growth, gltA was not continued with a further round of gene knockout. Adaptive laboratory evolution to increase growth or a less severe knockdown approach using CRISPRi30 are good candidate approaches to be attempted in the future. The knockout of aceA or gntZ on the XW15 strain, however, was still producing low levels of isoprenol: 12 mg/L for XW17 strain (ΔphaABC ΔmvaB ΔhbdH ΔaceA) and 13 mg/L for XW18 strain (ΔphaABC ΔmvaB ΔhbdH ΔgntZ), respectively (FIG. 19, b). In the final round of gene knockouts, the XW17 strain was used to integrate the gntZ deletion to create the XW19 strain (ΔphaABC ΔmvaB ΔhbdH ΔaceA ΔgntZ) that contains all gene knockouts that do not affect growth. Interestingly, the inclusion of gntZ knockout significantly restored isoprenol production to 114 mg/L (FIG. 19, b). Although this isoprenol level was still lower than the two previous strains (XW12 and XW15), it reflects the synergy among multiple gene knockout targets. To understand the different effects caused by ΔaceA and/or ΔgntZ, we compared the production profiles for XW17 to XW19 strains (FIG. 26). It was observed that the XW19 strain depleted glucose after 48 hours while the XW17 strain showed 6 g/L residual glucose in the medium. Correspondingly, the cell growth showed the opposite trends in these three strains, such that the XW19 strain showed the highest OD, which is 1.7-fold higher than that of the XW17 strain.

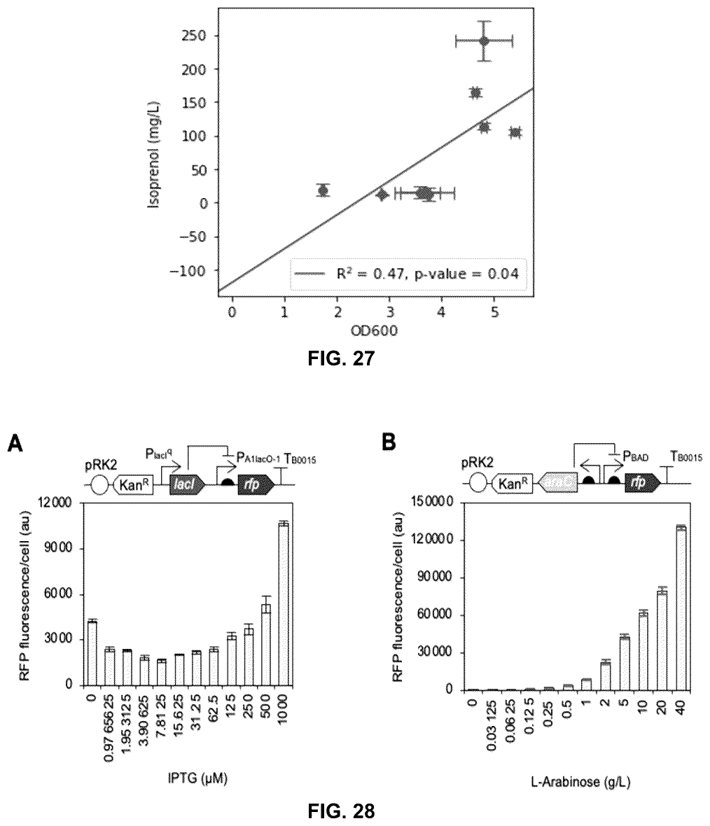

While the model requires knockout of all targets provided, we instead selected a subset of these targets as ranked by the frequency provided by several methods. The deletion of selected targets still showed significant improvement for isoprenol production even though the model does not predict growth-coupled isoprenol production with a subset. While a correlation analysis between isoprenol production and cell growth (OD₆₀₀) only showed a weak positive correlation (P<0.05, R²=0.47), it was observed that the higher producers usually showed better cell growth (FIG. 27). This suggested that the increased isoprenol production is not at the expense of cell growth at the current isoprenol levels. However, not all strains showed improved isoprenol production and it might be necessary to delete additional genes to achieve a higher yield. We also note that the model predictions were made using a minimal medium, whereas experiments were performed using EZ rich defined medium due to better isoprenol production19. Thus, optimizing the production performance in a minimal medium could be a prerequisite to further demonstrate the effectiveness of our frequency-based prioritization of gene targets. Nonetheless, these results show that the deletion of prioritized gene targets can lead to significant improvement in isoprenol production and thus provide support for our computational approach.

In summary, following the genome-scale metabolic modeling recommendations, we constructed single and multiple knockout P. putida mutant strains. Among the engineered knockout strains, a triple knockout mutant strain (XW15, ΔphaABC ΔmvaB ΔhbdH) showed the highest isoprenol production (241 mg/L) from EZ rich medium supplemented with 2% glucose. This demonstrated the utility of computational approaches for host strain optimization to achieve high titer, rate, and yield.

### Pathway Optimization for Improved Isoprenol Production

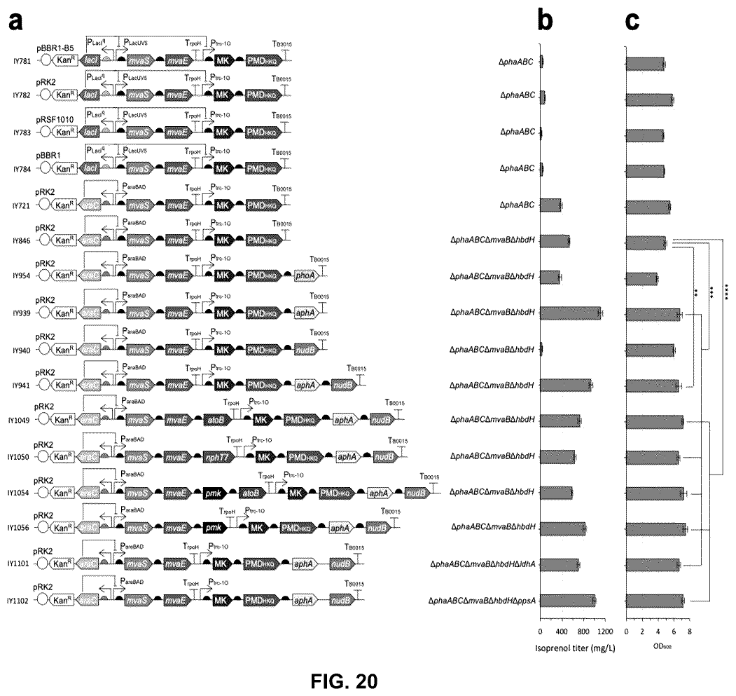

In parallel with the GSMM-guided metabolic rewiring efforts above, we continued to optimize isoprenol pathway gene expression to improve production in P. putida KT2440. We first expressed the IPP-bypass isoprenol biosynthetic pathway comprising mvaE, mvaS, mk, and pmduke in different plasmid backbones (FIG. 20, a; strains IY781-IY784, Table 5) under the control of a LacI repressor in the P. putida ΔphaABC strain (XW01). The highest titer of 80 mg/L isoprenol was achieved from strain IY782, carrying the isoprenol pathway in the plasmid with the RK2 replication origin (FIG. 20, b). Therefore, in the subsequent experiments, plasmid RK2 was used as the expression vector. Since we identified that the arabinose-inducible promoter (P_{BAD}) is a stronger promoter than PAllacO-1, (FIG. 28) we expressed the isoprenol biosynthetic pathway under the P_{BAD} promoter and replaced the LacI repressor with AraC, in an effort to improve isoprenol production. The absence of the LacI repressor resulted in the constitutive expression of MK and PMD_{HKQ} under a strong P_{tre-IO} promoter31. The resulting strain (IY721) produced up to 395 mg/L isoprenol in 48 hr (FIG. 20, b). When combined with the GSMM approaches by deleting two genes, mvaB and hbdH, the resulting strain (IY846) improved the isoprenol production by 1.3-fold vs. strain IY721, yielding approximately 536 mg/L isoprenol in 48 hr (FIG. 20, b).

TABLE 7
Minimal M9 medium recipe.
10 × M9 Salts

| Compound | Final concentrations |
|---|---|
| Na₂HPO₄ | 68 g |
| KH₂PO₄ | 30 g |
| NaCl | 5 g |

1 × minimal M9 medium solution

| Compound/Stock | Per 1 L | | Comments |
|---|---|---|---|
| 10 × M9 Salts | 100 | mL | Make 10 × stock/filter separately |
| 1M MgSO₄ | 2 | mL | Make 1M solution/filter separately |
| 1M CaCl₂ | 100 | μL | Make 1M solution/filter separately |
| MQ H₂O | 787.4 | mL | Autoclaved |
| 20% Glucose | 100 | ml | Autoclaved |
| Trace elements solution | 500 | μL | Teknova |
| (NH₄)₂SO₄ | 10 | ml | 1M stock |

Previous studies in E. coli demonstrated that phosphatase over-expression can boost isoprenol production. In our previous paper, we showed that NudB, a native phosphatase of E. coli, hydrolyzed IPP and DMAPP into their monophosphate forms, IP and DMAP, respectively, which are subsequently hydrolyzed to isoprenol by other phosphatases such as AphA, Agp, and YqaB15. Among these three phosphatases, AphA was found to best improve the isoprenol titer in E. coli15. A recent study for isoprenol production in S. cerevisiae, however, reported that co-expressing an E. coli alkaline phosphatase, PhoA, produced the highest isoprenol titer11. Therefore, we co-expressed NudB, PhoA, and AphA along with the isoprenol biosynthetic pathway and found that co-expressing AphA alone in the ΔphaABC ΔmvaB ΔhbdH background (strain IY939 with plasmid pIY670) produced the highest isoprenol titer of 1,111 mg/L in 48 hr (FIG. 20, b).

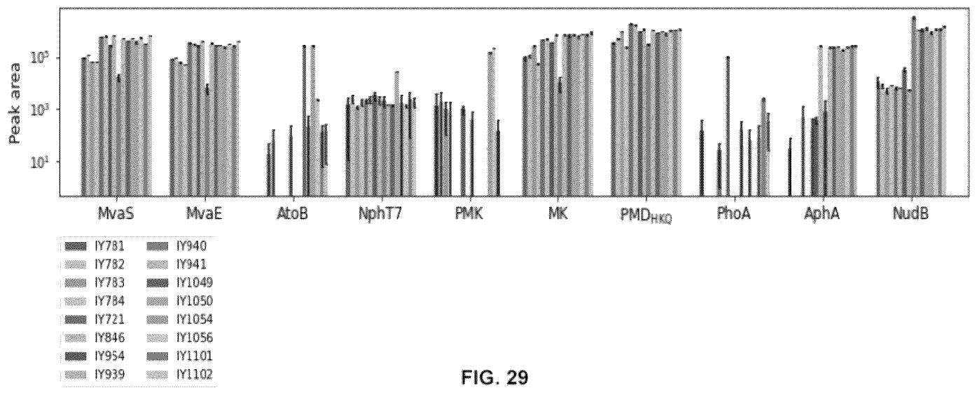

In an attempt to increase the acetyl-CoA pool, we co-expressed AtoB, NphT7, and/or PMK, and knocked out IdhA and ppsA. Targeted proteomics confirmed the expression of all proteins (FIG. 29), but none of the strains generated higher isoprenol titers compared to strain IY939 (FIG. 20, b). Strains co-expressing AphA also accumulated higher biomass compared to those not expressing AphA (FIG. 20, c).

### Isoprenol Production Using Optimized Isoprenol Production Pathway and Predicted Metabolic Rewiring in Glucose Minimal Medium

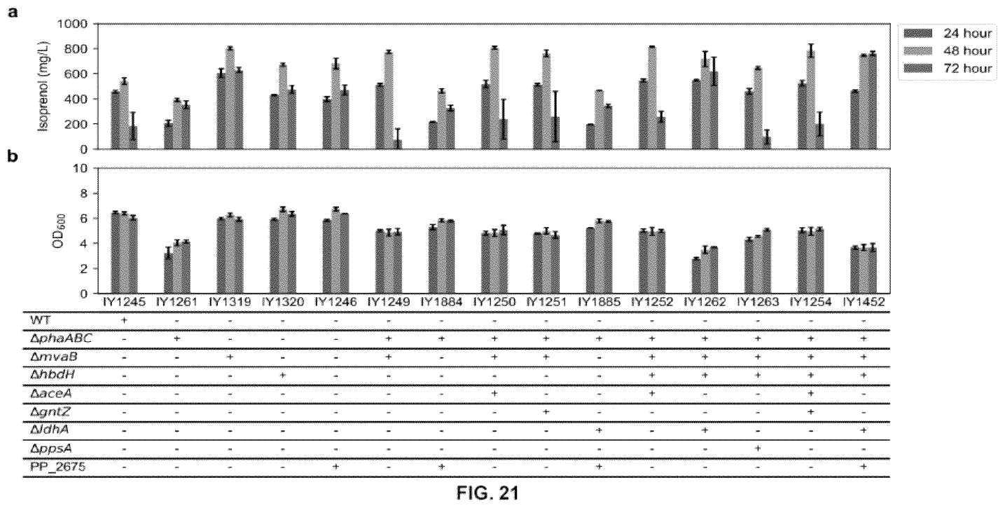

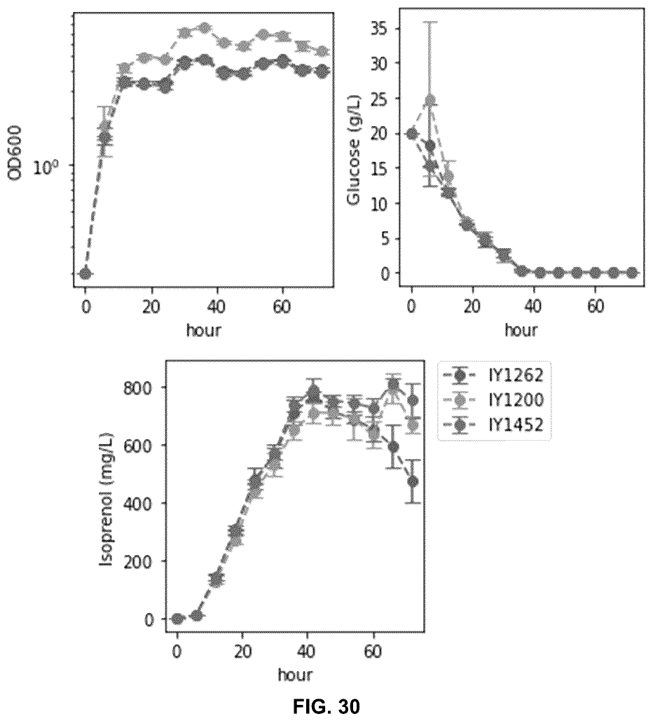

Using the optimized isoprenol pathway, we continued to characterize the engineered strains carrying GSMM-predicted gene knockouts. Defined rich medium such as EZ rich medium contains additional carbon and nitrogen sources (e.g. amino acids) that could trigger complex regulatory mechanisms, such as carbon catabolite repression32-34. The improved isoprenol production by the optimized pathway now enabled us to characterize the engineered strains in the minimal defined medium used for the GSMM-predicted gene targets. We tested wild-type and fourteen different knockout strains in the pIY670 background for growth and isoprenol production in M9 glucose minimal media plus 20 g/L glucose (FIG. 21). Since we switched from EZ rich medium (several carbon sources) to minimal medium with glucose as the sole carbon source, we deleted an additional gene, PP_2675, to avoid the catabolism of isoprenol after glucose consumption as this gene was reported to be involved in isoprenol degradation22. We observed that the IY1452 strain (ΔphaABC ΔmvaB ΔhbdH ΔldhA ΔPP_2675 with plasmid pIY670) had the best isoprenol titer at 762 mg/L, FIG. 21); over 4-fold higher than WT (IY1245). The highest isoprenol titer observed was 816 mg/L for the IY1252 strain at 48 h but that reduced by about 70% to 259 mg/L at 72 hr. We observed that there was negligible reduction in isoprenol titers in the IY1452 strain at 72 hr, unlike the other strains in M9 glucose minimal medium. Interestingly, the deletion of both IdhA and PP_2675 was needed to improve the maximum isoprenol production titer while reducing isoprenol degradation (FIG. 30).

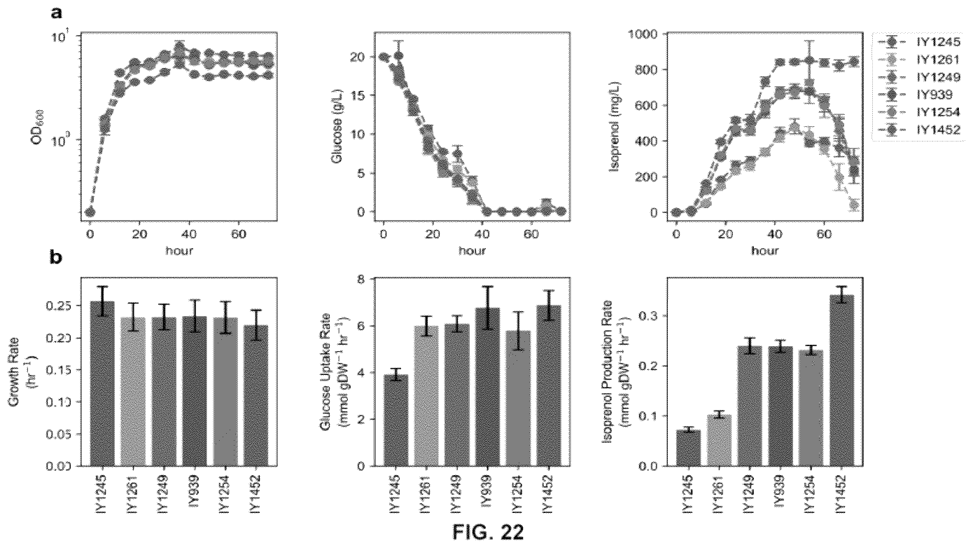

Next, we investigated the growth dynamics, carbon utilization, and isoprenol production profiles of the engineered strains via 72 hr time-course profiles of engineered strains in M9 glucose minimal medium. There was no statistical difference in growth rate, and glucose consumption only varied slightly (FIG. 22, a). IY1245 (base strain) and IY1261 had similar isoprenol production rates during the glucose consumption phase (up to 42 hrs) but strains IY1249, IY939, IY1254, and IY1452 had higher rates. IY1452 strain produced isoprenol at 0.34 mmol/gCDW/hr; a 4.69-fold improvement compared to the base strain. Importantly, IY1452 did not degrade isoprenol after glucose was depleted (FIG. 22, a).

### Improved Isoprenol Producing Phenotype Observed for the IY1452 Strain

Context-specific GSMMs were used to investigate the metabolic changes in the engineered strains using the constraints of the gene deletions and phenotypic data (glucose consumption, biomass formation, and isoprenol production rates, FIG. 22, b). To compare the metabolic changes between the different engineered strains and WT, through flux redistribution, we performed flux variability analysis for each context-specific GSMM. The metabolic flux span was calculated for each of the reactions during optimal growth under the defined constraints and normalized by the glucose uptake rate to compare the variability across different GSMMs. The flux span corresponds to the flexibility/rigidity of each reaction during maximal growth and isoprenol production based on experimental constraints. A narrow flux span means a rigid flux due to either a causal or correlational effect of the strain engineering for improved isoprenol production indicating very constrained metabolism at the given isoprenol production level and thus could lead to potential overexpression targets. A narrow flux may also be an effect of the engineering aimed at reducing the carbon flux away from the isoprenol pathway. On the contrary, a wider flux span reflects that these reactions have numerous permissible carbon flux values at the observed isoprenol production, and some of these reactions are potential deletion targets to further improve isoprenol production.

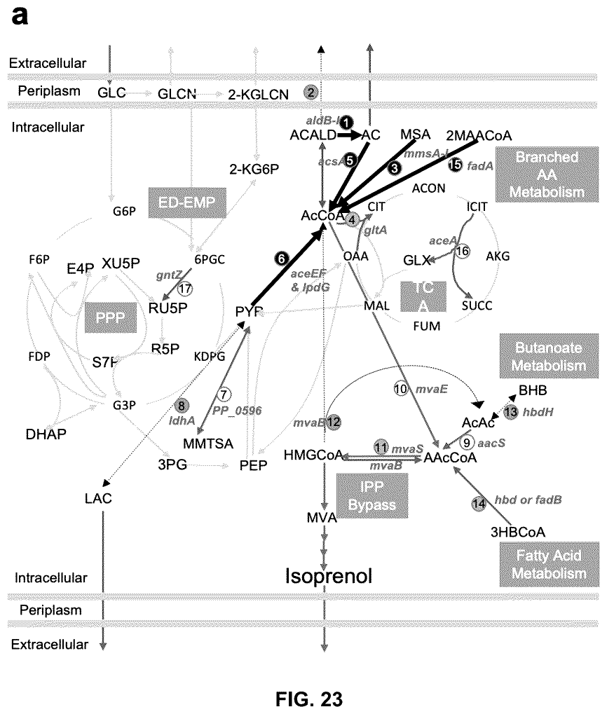

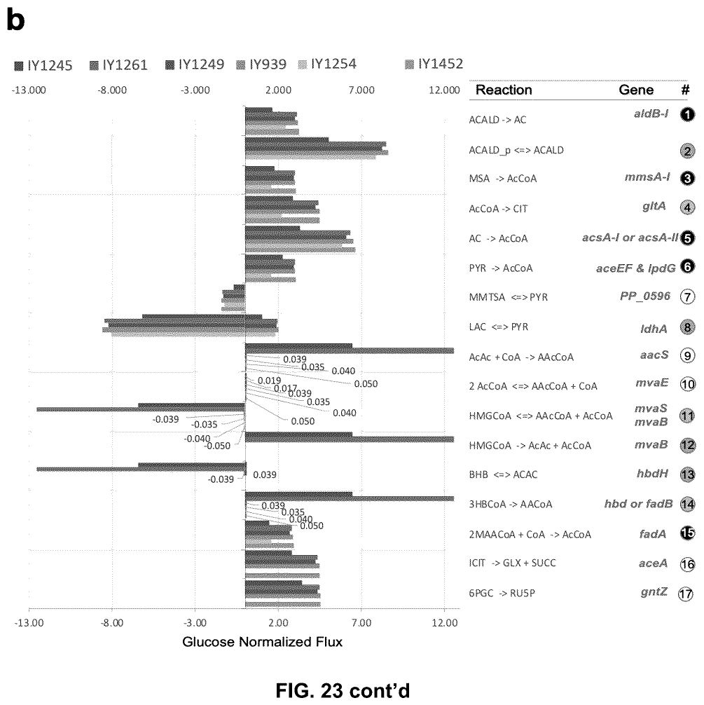

Selected reactions involving the precursor metabolites with respect to central metabolism and their flux spans are shown in FIG. 23. The flux variability analysis (FVA) of the context-specific GSMM shows that the second reaction in the heterologous IPP-bypass isoprenol production pathway, HMGCOAS (mvaS, Reaction 11 in FIG. 23), has a narrow flux span in the strains with additional gene deletions on top of ΔphaABC, i.e. strains that have the PP_3540/mvaB gene deletion. Given the constraints and assumptions in the GSMM, we can imagine two possible reasons for this narrow flux span in the ΔmvaB strains compared to the wider flux span in WT and ΔphaABC strains. It could be a result of high flux in competing pathways that redirect carbon flux away from the metabolic pathways of interest to maintain similar growth rates as WT and ΔphaABC strains (FIG. 22, b, 0.22 hr-1 versus 0.26 hr-1) given the limited resources available in M9 glucose minimal medium. Alternatively, the reduction in flux span in strains with mvaB deletion is perhaps due to the elimination of a critical degree of freedom, truly constraining the flux profile. Firstly using FVA, we observed a high flux in the HMG-COA consuming reaction, in case of WT and ΔphaABC strains (Reaction 12 in FIG. 23), a competing reaction that redirects carbon flux away from the heterologous IPP-bypass pathway. This high allowable flux was reduced by the PP_3540/mvaB deletion. Secondly, based on FVA, we observed there was a high flux span in BDH (hbdH, Reaction 13 in FIG. 23), in the direction diverting flux towards butanoate metabolism, away from the heterologous IPP-bypass pathway. This was resolved by the PP_3073/hbdH deletion on top of the mvaB deletion. In the GSMM, ACACTlr (Reaction 10 in FIG. 23) was assumed to have flux only in the direction of IPP-bypass due to additional pIY670 plasmid-borne mvaE activity. Zero flux through ACALDtpp (Reaction 2 in FIG. 23) represents reduction in secretion of byproducts such as acetaldehyde.

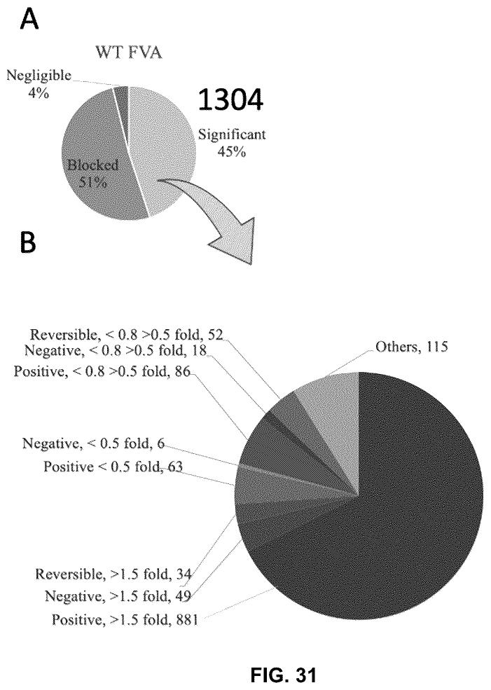

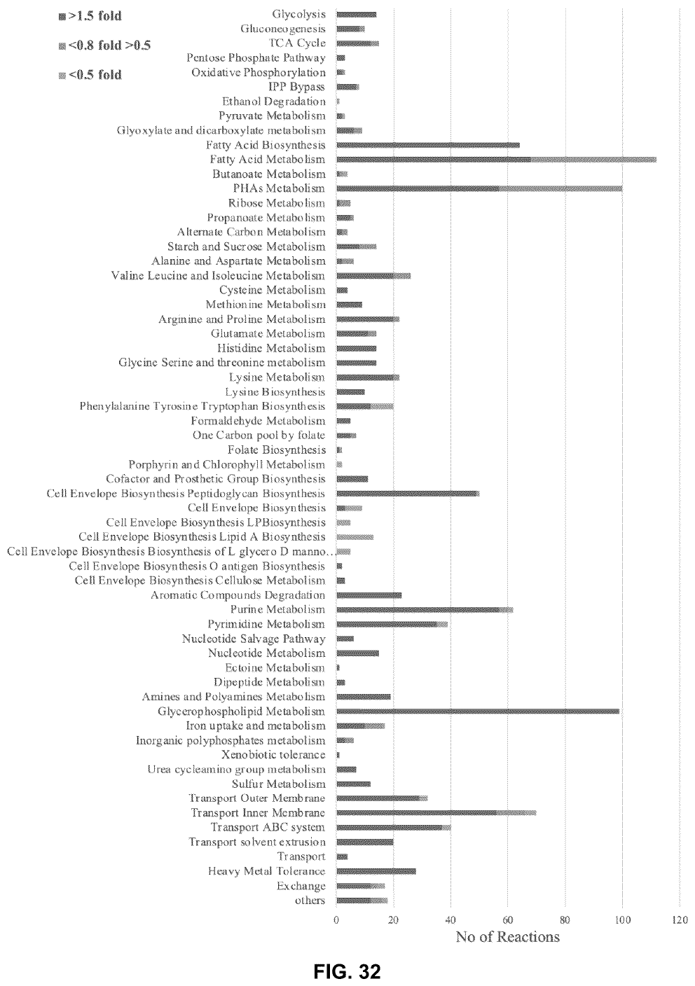

Although more than 50% of the reactions carried zero flux under glucose minimal medium conditions, 1,304 reactions (45%) carried a substantial flux (FIG. 31). After constraining the model using our experimental data, the best performing strain IY1452 showed substantial fold changes in fluxes although the directionality of the reactions was similar to WT (FIG. 32). Of these 1,304 reactions, 964 reactions had an increased flux span by more than 1.5-fold compared to WT and were spread across central carbon metabolism and biosynthesis pathways. There were 225 reactions with a reduced flux span ranging from 1% to 80% of WT flux magnitudes. 69 of these reactions had 0.5-fold or lower flux and mainly belong to alcohol degradation, butanoate metabolism, and also the HMG-COA synthase reaction of the IPP-bypass pathway. Additionally, 156 reactions had a flux span reduction between 0.5-fold to 0.8-fold and were shared across non-unique subsystems including alanine and aspartate metabolism, branched amino acid metabolism, fatty acid metabolism, and PHA metabolism.

In summary, when compared to WT, the best performing strain IY1452 showed increased flux through desirable reactions for an increased acetyl-CoA pool (FIG. 23, b, reactions 1, 3, 5, 6, and 15) and reduced to negligible flux through competing reactions that redirect carbon flux away from the isoprenol production pathway (FIG. 23, b, reactions 2, 8, 12 and 13). A restricted flux span through reactions 11 and 14 points toward future strain engineering to redirect flux from fatty acid metabolism towards HMG-COA for further improving isoprenol production. A high flux span through citrate synthase (Reaction 4 in FIG. 23) in most of the engineered strains suggests revisiting gltA as a target for down-regulation. Although the gltA deletion showed a significant growth defect, it was one of the top targets predicted by our computational approach (Table 4) and also previously identified by kinetic modeling as a down-regulation target to increase acetyl-CoA availability in P. putida30.

### Isoprenol Production in Fed-Batch Cultivation

Four strains (IY1245 (control), IY1262, IY1452, and IY1485) were cultured in fed-batch mode to increase isoprenol titer by supplying additional carbon and nitrogen. After the batch phase with the modified M9 minimal medium containing 20 g/L of glucose and 1.06 g/L (or 20 mM) ammonium chloride, the feeding solution was continuously added to make a total of 100 g/L glucose and 2.12 g/L ammonium chloride. As isoprenol evaporates rapidly due to airflow in the bioreactor9, the exhaust line was vented through a bottle containing 1 L oleyl alcohol as a capture solvent to extract isoprenol from the off-gas.

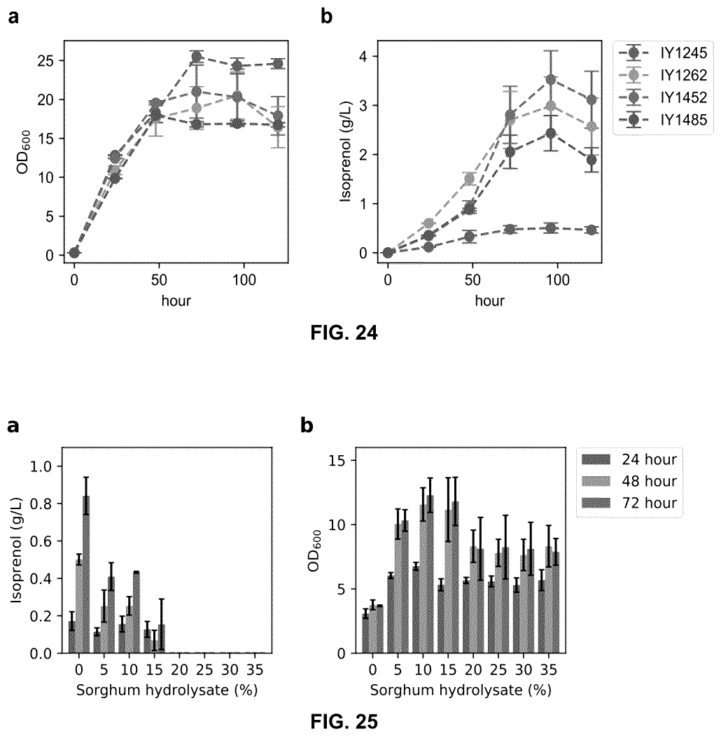

In the IY1245 control strain, the maximum cell growth and the isoprenol production were obtained at 72 hr, reaching an OD₆₀₀ of 25.5±0.7 and isoprenol titer of 0.5±0.1 g/L, respectively (FIG. 24). The initial 20 g/L glucose was depleted by 14 hr and the isoprenol production was revealed from the off-gas after 24 hr, but interestingly, no isoprenol was detected from the culture extract by then (data not shown). This suggests that most isoprenol produced was evaporated by airflow as previously reported in the E. coli study9. The IY1262 strain produced 2.3±0.3 g/L of isoprenol and the OD₆₀₀ reached 20.5±3.0 at 96 hr (FIG. 24). The growth rate of the engineered strain was slower than the wild-type strain, but the titer was significantly increased. The maximum growth and isoprenol production on the IY1452 strain were obtained, reaching an OD₆₀₀ of 21.0±3.4 at 72 hr and a titer of 3.5±0.3 g/L at 96 hr (FIG. 24).

Aeration is required in P. putida cultivation, but it resulted in a strong foaming, which was difficult to handle even with conventional antifoams. Furthermore, the excessive foam hinders the use of a standard cultivation protocol35,36. To reduce foaming during the fed-batch cultivation, the gacA gene was deleted on the 5 genes knockout strain (IY1452) as previously reported37. The resulting IY1485 strain reached the OD₆₀₀ of 17.9±0.2 at 48 hr and produced 2.4±0.3 g/L of isoprenol at 96 hr in fed-batch mode. Even though the gacA gene knockout resulted in a significant reduction of foaming, it also resulted in slower growth and lower isoprenol production than the other mutants.

### Isoprenol Production Using Biomass Hydrolysate

The use of lignocellulosic biomass for the production of biofuels and bioproducts is of increasing interest38 and P. putida is widely recognized for this purpose39,40. Therefore, we evaluated the production of isoprenol by strain IY1452 using a modified M9 minimal medium supplemented with glucose or sorghum hydrolysate as the carbon source. The highest isoprenol titer from this strain was 841 mg/L at 72 hr in a modified M9 minimal medium supplemented with 20 g/L of glucose as a sole carbon source (FIG. 25, a). Isoprenol production was lower with sorghum hydrolysate: 409 mg/L and 432 mg/L at 72 hr with 5% and 10% sorghum hydrolysate, respectively (FIG. 25, a). However, it is noteworthy that the growth rates of cultures with sorghum hydrolysate were higher than the growth rate with pure glucose, suggesting additional nutrients in the hydrolysate promoted cell growth (FIG. 25, b). Despite the lower isoprenol titers, the culture with sorghum hydrolysate showed promise as an alternative production medium with a higher growth rate than the culture with glucose as the sole carbon source.

Our GSMM-based computational strain design predictions were based on glucose as the sole carbon source under minimal medium cultivation conditions. Sorghum-based hydrolysates are composed of a variety of carbon sources that are further dependent on the pretreatment method10,41,42. It is reported that Sorghum-based ionic liquid ([Ch][Lys]) pretreated hydrolysate consists of glucose, xylose, acetate, and several aromatic compounds 10. P. putida KT2440 lacks the capability to utilize xylose natively but has been reportedly engineered for xylose utilization43,44. We observed improvement in growth across all tested fractions of hydrolysate but the isoprenol titers decreased with increasing fraction of hydrolysate in the medium when compared to glucose as the sole carbon source. This can be attributed to the presence of multiple carbon re-routing metabolic pathways towards growth versus limited bioconversion routes towards isoprenol production via the IPP-bypass pathway.

### CONCLUSIONS

Anthropogenic release of carbon into the atmosphere has resulted in climate change, and sustainable aviation fuels (SAFs) offer an effective near-term means of mitigating this continued deleterious carbon release. In this study, we have reported our efforts to engineer strains of Pseudomonas putida that can produce the SAF precursor isoprenol from plant-derived carbon sources. We simultaneously pursued rational and GSMM-based target selection approaches followed by engineering and testing in various culture configurations, including fed-batch bioreactors.

Two GSMM approaches were applied and each predicted a significant number of gene knockout targets in order to realize the computationally predicted improvement in isoprenol yield. Through an ensemble ranking of the myriad gene targets from the two approaches, we were able to prioritize and reduce the total number of targets. This approach proved fruitful in decreasing the number of engineered strains needed to realize a significant improvement in titer and rate. However, we also observed that some of the predictions did not result in titer improvements, and some combinations of knockouts were detrimental to P. putida growth and/or isoprenol titers. Rational pathway optimization had a significant impact on titer improvement. The synergistic application of GSMM-guided gene knockouts and rational pathway optimization led to the highest titer of isoprenol in P. putida at 1.1 g/L; a 10-fold improvement vs. the starting strain. Fed-batch cultivation further improved the titer to 3.5 g/L.

Since knocking-out multiple genes in P. putida is not a trivial amount of effort, and the knock-outs frequently result in growth retardation, gene knock-downs could be an alternative to gene knock-out to screen multiple combinations of target genes. Application of CRISPR interference and building an automated process may accelerate rapid strain engineering to improve isoprenol TRY. Further, adaptive laboratory evolution (ALE)-based tolerization41 and other state-of-the-art strain engineering techniques44,45 can be applied to further improve isoprenol titers, rates, and yields in future research. For ultimate industrial applications, additional improvements must be made, including further genetic engineering strain improvements, bioprocess optimization, and separations process engineering to include downstream recovery of the volatile product.

### Methods

Computation of Constrained Minimal Cut Sets (cMCS) and Elementary Modes

Pseudomonas putida KT2440 genome scale metabolic model (GSMM) iJN146224 was used. Aerobic conditions with glucose as the sole carbon source were used to model growth parameters. The ATP maintenance demand and glucose uptake were 0.97 mmol ATP/gDW/h and 6.3 mmol glucose/gDW/h, respectively. Constrained minimal cut sets (cMCS) were calculated using the MCS algorithm26 available as part of CellNetAnalyzer (version 2020.2) 46. Excretion of byproducts was initially set to zero, except for the reported overflow metabolites for secreted products specific to P. putida (gluconate, 2-ketogluconate, 3-oxoadipate, catechol, lactate, methanol, CO2, and acetate). We calculated the maximum theoretical yields (MTY) for isoprenol using glucose as the carbon source and the heterologous IPP bypass pathway (0.72 mol/mol of glucose). Additional inputs including minimum demanded product yield (10% to 85% of MTY) and maximum demanded biomass yield at 10 to 25% of maximum biomass yield were also specified in order to constrain the desired design space. The maximum size of MCS was kept at the default (i.e. 50 metabolic reactions). Knockouts of export reactions and spontaneous reactions were not allowed. With the specifications used herein, each calculated knockout strategy (cMCS) demands production of isoprenol even when cells do not grow. All cMCS calculations were done using API functions of CellNetAnalyzer46 on MATLAB 2017b platform using CPLEX 12.8 as the MILP solver. The different runs, respective number of cut sets and number of targeted reactions to satisfy coupling constraints are included in Supplementary File 3.

For elementary modes computation, a small model representing the central carbon metabolism of Pseudomonas putida KT2440 and the heterologous IPP bypass isoprenol production pathway was used to calculate elementary modes by efintool25.

### Prediction of Gene Targets Using Opt-Based Methods

The iJN1462 metabolic model was also used for OptKnock and OptForce. The model was first modified to fix mass or charge unbalanced reaction, remove duplicate reactions involving lipoamide dehydrogenase, remove the reactions catalyzed by genes on the TOL plasmid pWWO, remove the PPCK reaction by a pseudogene phosphoenolpyruvate carboxykinase PP_0253, and update the gene-protein-reaction association for the OAADC reaction from 2-dehydro-3-deoxy-phosphogluconate aldolase PP_1024 to oxaloacetate decarboxylase PP_1389. The modified model was augmented with the IPP-bypass pathway for isoprenol production (Supplementary File 1).

For OptKnock, the model was preprocessed to remove blocked reactions and metabolites and identify the reactions predicted to be essential for growth on glucose as a sole carbon source. The predicted essential reactions, spontaneous reactions, boundary reactions, and periplasmic transport reactions without associated genes were excluded from knockout targets. Several additional reactions were manually excluded from knockout targets to avoid undesired predictions (ATPM, CAT, CYO1_KT, CYTBO3_4pp, CYTCAA3pp, NADH16pp, NAt3_1p5pp, PItex, and PPK). The OptKnock problem was constructed using cobrapy47 and solved using CPLEX 12.8. Several iterations of OptKnock were run to identify a large number of knockout strategies using the solution pool and integer cuts. Another set of OptKnock solutions were obtained using a further constrained model where the secretion of other byproducts was blocked except for gluconate, 2-ketogluconate, and acetate assuming no significant byproduct formation (Supplementary File 4).

For OptForce, the model was also preprocessed to remove blocked reactions in glucose minimal media condition. The flux ranges for wild type were obtained by running flux variability analysis with constraints on glucose uptake, gluconate secretion, glucose dehydrogenase, gluconokinase, phosphogluconate dehydratase, pyruvate dehydrogenase, and citrate synthase taken from a previous study48. For isoprenol overproduction, we used 50% of the theoretical maximum production as a pre-specified target to identify designs. All possible first and second-order necessary flux changes for overproduction were first identified and then used to identify the minimum set of interventions including flux increase, decrease, or knockouts. Several iterations of OptForce were run to identify a large number of designs by adding integer cuts using CPLEX 12.8 (Supplementary File 4).

### Context Specific Models and Flux Variability Analysis

For flux variability analysis, first context-specific models were generated using constraints derived from experimental data to create six different P. putida GSMMs to represent the 6 different P. putida strains engineered for isoprenol production in this study. Constraints for glucose uptake rate, isoprenol production rate as well as growth rate were used. Next we performed flux variability analysis using fluxvariabilityanalysis( ) function in the COBRA Toolbox49 on the MATLAB 2017b platform.

### Strains and Plasmid Construction

All strains and plasmids used in this study are listed in Table 6. Strains and plasmids along with their associated information have been deposited in the public domain of the JBEI Registry (website for: public-registry.jbei.org) and are available from the authors upon request. Gene knockout of P. putida was performed based on the homologous recombination followed by a suicide gene (sacB) counter-selection as described50. The genotypes of gene-knockout mutants were confirmed by colony PCR using specific primers, followed by DNA sequencing (GENEWIZ, South San Francisco, CA, USA).

Isoprenol production in P. putida

P. putida KT2440 strains bearing isoprenol pathway plasmids (Supplementary Table 6) were used for isoprenol production. Starter cultures of all production strains were prepared by growing single colonies in LB medium containing 50 μg/mL kanamycin at 30° C. with 200 rpm shaking overnight. The starter cultures were diluted in 5 mL EZ rich defined medium (Teknova, CA, USA) containing 20 g/L glucose (2%, w/v), 25 μg/mL kanamycin in 50-mL test tubes, and 0.5 mM IPTG or Arabinose (2%) was added to induce protein expression with OD₆₀₀ at 0.4-0.6. The P. putida cultures were incubated in rotary shakers (200 rpm) at 30° C. for 48 hr.

For isoprenol production runs on M9 minimal medium (Table 7) with 2% D-glucose, cryostocks were streaked to singles on LB agar plate with 50 μg/mL kanamycin at 30° C. Single colonies were inoculated and grown overnight with shaking in 5 mL liquid LB medium supplemented with 50 μg/mL kanamycin at 30° C. and 200 rpm. Unless otherwise mentioned, all further cultivations were performed in the same format and conditions. 100 μL of these overnight LB grown cultures were back diluted into the minimal medium and grown for 24 hr. A second back dilution enabled complete adaptation in the minimal medium. For the production runs, the cells were inoculated at an initial OD₆₀₀ of 0.2 and the isoprenol production pathway was induced with 2% Arabinose immediately after inoculation. Samples were collected every 6 hr until 72 hr in triplicates and analyzed for growth (OD₆₀₀), isoprenol, residual glucose and organic acids.

The quantification of isoprenol was conducted as described in Kim et al., 2021 11. Briefly, 100 μL of ethyl acetate containing 1-butanol (30 mg/L) as the internal standard was added to 100 μL of liquid cultures. The mixture was vortexed at 3000 rpm for 15 min and subsequently centrifuged at 21,130×g for 3 min to separate the ethyl acetate phase from the aqueous phase. 1 μL of the ethyl acetate layer was analyzed by gas chromatography-flame ionization detection (GC-FID, Thermo Focus GC) equipped with a DB-WAX column (15 m, 0.32 mm inner diameter, 0.25 μm film thickness, Agilent, USA). The GC oven was programmed as follows: 40° C. to 100° C. at 15° C./min, 100° C. to 230° C. at 40° C./min finally, held at 230° C. for 2 min. The inlet temperature was 200° C. Serial dilutions of isoprenol were prepared to determine the quantification of isoprenol in the samples.

The residual glucose and organic acids were analyzed using high performance liquid chromatography (HPLC, Agilent, USA) equipped with a refractive index detector (RID) and an Aminex HPX-87X column (Bio-Rad, USA) with 4 mM sulfuric acid as the mobile phase in the isocratic mode. The following conditions were used: Mobile phase flow rate: 0.6 mL/min, column at 60° C., RID at 35° C. Serial dilutions of glucose and organic acids were used to determine the concentration of glucose and organic acids in the samples. Data analysis was carried out on the ChemStation software (Agilent Technologies).

### Targeted Proteomics Analysis of the Isoprenol Biosynthesis Pathway Proteins

Cell pellets of the engineered P. putida strains for isoprenol production were prepared for targeted proteomic analysis according to Chen et al51. Briefly, cells were resuspended in a solution with 80 μL of methanol and 20 μL of chloroform and thoroughly mixed by pipetting. Sixty microliters of water were subsequently added to the samples and mixed. Phase separation was induced with 5 minutes of centrifugation at 1000×g. The methanol and water layers were removed, and then methanol (80 μL) was added to each well. The plate was centrifuged for 1 minute at 100×g, and then the supernatant layers were decanted. The protein pellets were resuspended in a 100 mM ammonium bicarbonate buffer supplemented with 20% methanol, and the protein concentration was determined by the DC assay (Bio-Rad). proteins from each sample were reduced by addition of tris 2-(carboxyethyl) phosphine to 5 mM for 30 min at room temperature and followed by alkylation with iodoacetamide at 10 mM for 30 min at room temperature in the dark. Protein digestion with trypsin at 1 g/L concentration was accomplished with a 1:50 (w/w) trypsin/total protein ratio overnight. The multiple-reaction monitoring (MRM) assay was developed for relative quantification of isoprenol biosynthesis pathway proteins through a rapid method development workflow established previously52. Targeted proteomic analysis was performed on an Agilent 1290 UHPLC system coupled to an Agilent 6460 QqQ mass spectrometer according to an established protocol (webpage for: dx.doi.org/10.17504/protocols.io.bf9xjr7n). Briefly, 20 g Peptides of each sample were separated on an Ascentis Express Peptide C18 column [2.7-mm particle size, 160-Å pore size, 5-cm length×2.1-mm inside diameter (ID), coupled to a 5-mm×2.1-mm ID guard column with the same particle and pore size, operating at 60° C.; Sigma-Aldrich] operating at a flow rate of 0.4 ml/min via the following gradient: initial conditions were 98% solvent A (0.1% formic acid), 2% solvent B (99.9% acetonitrile, 0.1% formic acid). Solvent B was increased to 5% over 1 min, and was then increased to 40% over 3.5 min. It was increased to 80% over 0.5 min and held for 2.5 min at a flow rate of 0.6 mL/min, followed by a linear ramp back down to 2% B at a flow rate of 0.4 mL/min over 0.5 min where it was held for 1 min to re-equilibrate the column to original conditions. The eluted peptides were ionized via an Agilent Jet Stream ESI source operating in positive ion mode. The MS raw data were acquired using Agilent MassHunter version B.08.02, and were analyzed by Skyline software version 21.20 (MacCoss Lab Software). The MRM method and data are available at Panoramaweb53 (website for: panoramaweb.org/genome-scale-eng-SAF-p-putida.url) and at ProteomeXchange via identifier PXD039868.

### Isoprenol Production in Fed-Batch Mode

For the isoprenol production in fed-batch mode, the strains were cultured in 5 mL LB medium with 50 μg/mL kanamycin at 30° C. For the adaptation, the cell culture was diluted 50-fold in the fresh 5 mL modified M9 minimal medium two times. Then the seed culture was inoculated in the 1 L baffled flask including 100 mL modified M9 minimal medium at 30° C. and 200 rpm for 8 hr. The cell culture was inoculated at OD₆₀₀ 0.3 in the 2 L bioreactor (Biostat B, Sartorius, Germany) including the 1 L modified M9 minimal medium, which contained 6.8 g/L Na₂HPO₄, 3.0 g/L KH₂PO₄, 0.5 g/L NaCl, 20 mM NH₄Cl, 2 mM MgSO₄, 0.1 mM CaCl₂) and trace metal solution. The 1000× trace metal solution (TEKNOVA, USA) consisted of 50 mM FcCl₃, 20 mM CaCl₂), 10 mM MnCl₂, 10 mM ZnSO₄, 2 mM CoCl₂, 2 mM CuCl₂, 2 mM NiCl₂, 2 mM Na₂MoO₄, 2 mM Na₂O₃Se, and 2 mM BH₃O₃. To produce isoprenol in the fed-batch fermentation, the dissolved oxygen (DO) and airflow were set to 20% and 1 VVM (volume of air per volume of liquid per minute), respectively. The temperature was maintained 25° C. and pH was maintained at 6.5 by supplementation with 25% ammonia water. The isoprenol biosynthesis pathway was induced at OD₆₀₀ 0.6-0.8 by 2% arabinose. The antifoam B was added to the bioreactor when required. To feed the additional carbon and nitrogen sources, a total of 80 g glucose and 15 g ammonium chloride in solution was continuously supplied using a Watson-Marlow DU520 peristaltic pump. The feeding flow rate was set to closely match the glucose consumption rate at the end of the batch phase. After the lag phase, the feeding flow rate was calculated following Korz's equation and increased every hour for a total of 6 hr 9,54.

$$μ ( t ) = {μ}_{set} + \frac{1}{t - {t}_{F}} ln \frac{V ( t )}{{V}_{F}}$$

For the exponential feeding, the glucose in the media was measured consistently using the glucose meter (CVSHealth, USA) and high-performance liquid chromatography (HPLC), and feeding rate was set constant in order that the concentration of glucose was dropped below than 1 g/L in the medium. To extract the isoprenol from the off gas, the exhaust line was connected directly to a bottle including the 1 L oleyl alcohol as extraction solvent. For quantification of isoprenol, 10 μL of oleyl alcohol layer was added to 990 mL of ethyl acetate containing 1-butanol (30 mg/L) as internal standard.

### Isoprenol Production Using Biomass Hydrolysate

For the isoprenol production using biomass hydrolysate, the cholinium lysinate ionic liquid-pretreated sorghum hydrolysate was obtained from Joint BioEnergy Institute (JBEI) Deconstruction Division55 and was used as a carbon source for the engineered P. putida strain. The sorghum hydrolysate was adjusted at pH 6.5 by sodium hydroxide and supplemented with 10× modified M9 salts at varying concentrations (0%, 5%, 10%, 15%, 20%, 25%, 30%, and 35% (v/v)). The glucose was added either as a sole carbon source or as co-substrate with sorghum hydrolysate and its concentration was adjusted to 20 g/L. Subsequently, we added 10 μL of 1 M MgSO₄, 5 μL of 100 mM CaCl₂), 2.5 μL of trace metal solution, and 50 μL of 1M NH₄Cl to the modified M9 minimal medium. And the modified M9 minimal medium volume was adjusted to 5 mL. The strains were cultured in 5 mL LB medium with 50 μg/mL kanamycin at 30° C. overnight. To adapt the strain, the cell culture was diluted 50-fold in the fresh 5 mL modified M9 minimal medium two times. Then the seed culture was inoculated in the culture tubes including 5 mL modified M9 minimal medium supplemented sorghum hydrolysate at 30° C. and 200 rpm. The isoprenol biosynthesis pathway was induced at OD₆₀₀ 0.6-0.8 by 2% arabinose. The isoprenol extraction was carried out using the same protocols as described above.

### REFERENCES CITED IN EXAMPLE 2

1. Baral, N. R. et al. Techno-economic analysis and life-cycle greenhouse gas mitigation cost of five routes to bio-jet fuel blendstocks. Energy Environ. Sci. 12, 807-824 (2019).
2. Keasling, J. et al. Microbial production of advanced biofuels. Nat. Rev. Microbiol. 19, 701-715 (2021).
3. Liew, F. E. et al. Carbon-negative production of acetone and isopropanol by gas fermentation at industrial pilot scale. Nat. Biotechnol. 40, 335-344 (2022).
4. Cruz-Morales, P. et al. Biosynthesis of polycyclopropanated high energy biofuels. Joule (2022) doi: 10.1016/j.joule.2022.05.011.
5. Geiselman, G. M. et al. Conversion of poplar biomass into high-energy density tricyclic sesquiterpene jet fuel blendstocks. Microb. Cell Fact. 19, 208 (2020).
6. Liu, C.-L. et al. Renewable production of high density jet fuel precursor sesquiterpenes from Escherichia coli. Biotechnol. Biofuels 11, 285 (2018).
7. Department of Energy, U.S. Co-Optimization of Fuels & Engines Year in Review (FY20). (2021) doi: 10.2172/1782424.
8. Baral, N. R. et al. Production Cost and Carbon Footprint of Biomass-Derived Dimethylcyclooctane as a High-Performance Jet Fuel Blendstock. ACS Sustain. Chem. Eng. 9, 11872-11882 (2021).
9. Kang, A. et al. Optimization of the IPP-bypass mevalonate pathway and fed-batch fermentation for the production of isoprenol in Escherichia coli. Metab. Eng. 56, 85-96 (2019).
10. Sasaki, Y. et al. Engineering Corynebacterium glutamicum to produce the biogasoline isopentenol from plant biomass hydrolysates. Biotechnol. Biofuels 12, 41 (2019).
11. Kim, J. et al. Engineering Saccharomyces cerevisiae for isoprenol production. Metab. Eng. 64, 154-166 (2021).
12. Baral, N. R. et al. Approaches for more efficient biological conversion of lignocellulosic feedstocks to biofuels and bioproducts. ACS Sustain. Chem. Eng. 7, 9062-9079 (2019).
13. Weimer, A., Kohlstedt, M., Volke, D. C., Nikel, P. I. & Wittmann, C. Industrial biotechnology of Pseudomonas putida: advances and prospects. Appl. Microbiol. Biotechnol. 104, 7745-7766 (2020).
14. Martínez-García, E. & de Lorenzo, V. Pseudomonas putida in the quest of programmable chemistry. Curr. Opin. Biotechnol. 59, 111-121 (2019).
15. Kang, A. et al. Isopentenyl diphosphate (IPP)-bypass mevalonate pathways for isopentenol production. Metab. Eng. 34, 25-35 (2016).
16. Tian, T., Kang, J. W., Kang, A. & Lee, T. S. Redirecting Metabolic Flux via Combinatorial Multiplex CRISPRi-Mediated Repression for Isopentenol Production in Escherichia coli. ACS Synth. Biol. 8, 391-402 (2019).
17. Hernandez-Arranz, S., Perez-Gil, J., Marshall-Sabey, D. & Rodriguez-Concepcion, M. Engineering Pseudomonas putida for isoprenoid production by manipulating endogenous and shunt pathways supplying precursors. Microb. Cell Fact. 18, 152 (2019).
18. Gao, X. et al. Engineering the methylerythritol phosphate pathway in cyanobacteria for photosynthetic isoprene production from CO2. Energy Environ. Sci. 9, 1400-1411 (2016).
19. Wang, X. et al. Engineering isoprenoids production in metabolically versatile microbial host Pseudomonas putida. Biotechnol. Biofuels Bioprod. 15, 137 (2022).
20. Rand, J. M. et al. A metabolic pathway for catabolizing levulinic acid in bacteria. Nat. Microbiol. 2, 1624-1634 (2017).
21. Thompson, M. G. et al. Massively parallel fitness profiling reveals multiple novel enzymes in Pseudomonas putida lysine metabolism. MBio10, (2019).
22. Thompson, M. G. et al. Fatty acid and alcohol metabolism in Pseudomonas putida: functional analysis using random barcode transposon sequencing. Appl. Environ. Microbiol. 86, (2020).
23. Maia, P., Rocha, M. & Rocha, I. In Silico Constraint-Based Strain Optimization Methods: the Quest for Optimal Cell Factories. Microbiol. Mol. Biol. Rev. 80, 45-67 (2016).
24. Nogales, J. et al. High-quality genome-scale metabolic modelling of Pseudomonas putida highlights its broad metabolic capabilities. Environ. Microbiol. 22, 255-269 (2020).
25. Terzer, M. & Stelling, J. Large-scale computation of elementary flux modes with bit pattern trees. Bioinformatics 24, 2229-2235 (2008).
26. von Kamp, A. & Klamt, S. Growth-coupled overproduction is feasible for almost all metabolites in five major production organisms. Nat. Commun. 8, 15956 (2017).
27. Burgard, A. P., Pharkya, P. & Maranas, C. D. Optknock: a bilevel programming framework for identifying gene knockout strategies for microbial strain optimization. Biotechnol. Bioeng. 84, 647-657 (2003).
28. Ranganathan, S., Suthers, P. F. & Maranas, C. D. OptForce: an optimization procedure for identifying all genetic manipulations leading to targeted overproductions. PLOS Comput. Biol. 6, e1000744 (2010).
29. Segrè, D., Vitkup, D. & Church, G. M. Analysis of optimality in natural and perturbed metabolic networks. Proc Natl Acad Sci USA 99, 15112-15117 (2002).
30. Kozaeva, E. et al. Model-guided dynamic control of essential metabolic nodes boosts acetyl-coenzyme A-dependent bioproduction in rewired Pseudomonas putida. Metab. Eng. 67, 373-386 (2021).
31. Cook, T. B. et al. Genetic tools for reliable gene expression and recombineering in Pseudomonas putida. J. Ind. Microbiol. Biotechnol. 45, 517-527 (2018).
32. Browne, P., Barret, M., O'Gara, F. & Morrissey, J. P. Computational prediction of the Crc regulon identifies genus-wide and species-specific targets of catabolite repression control in Pseudomonas bacteria. BMC Microbiol. 10, 300 (2010).
33. Moreno, R., Martínez-Gomariz, M., Yuste, L., Gil, C. & Rojo, F. The Pseudomonas putida Crc global regulator controls the hierarchical assimilation of amino acids in a complete medium: evidence from proteomic and genomic analyses. Proteomics 9, 2910-2928 (2009).
34. Molina, L., La Rosa, R., Nogales, J. & Rojo, F. Influence of the Crc global regulator on substrate uptake rates and the distribution of metabolic fluxes in Pseudomonas putida KT2440 growing in a complete medium. Environ. Microbiol. 21, 4446-4459 (2019).
35. Blesken, C. C. et al. Genetic Cell-Surface Modification for Optimized Foam Fractionation. Front. Bioeng. Biotechnol. 8, 572892 (2020).
36. Vo, M. T., Ko, K. & Ramsay, B. Carbon-limited fed-batch production of medium-chain-length polyhydroxyalkanoates by a phaZ-knockout strain of Pseudomonas putida KT2440.J. Ind. Microbiol. Biotechnol. 42, 637-646 (2015).
37. Eng, T. T. et al. High Throughput Fitness Profiling Reveals Loss Of GacS-GacA Regulation Improves Indigoidine Production In Pseudomonas putida. BioRxiv (2021) doi: 10.1101/2021.02.02.429437.
38. Mohammad, S. H. & Bhukya, B. Biotransformation of toxic lignin and aromatic compounds of lignocellulosic feedstock into eco-friendly biopolymers by Pseudomonas putida KT2440. Bioresour. Technol. 363, 128001 (2022).
39. Sodré, V., Vilela, N., Tramontina, R. & Squina, F. M. Microorganisms as bioabatement agents in biomass to bioproducts applications. Biomass and Bioenergy 151, 106161 (2021).
40. Linger, J. G. et al. Lignin valorization through integrated biological funneling and chemical catalysis. Proc Natl Acad Sci USA 111, 12013-12018 (2014).
41. Lim, H. G. et al. Generation of ionic liquid tolerant Pseudomonas putida KT2440 strains via adaptive laboratory evolution. Green Chem. 22, 5677-5690 (2020).
42. Park, M. et al. Response of Pseudomonas putida to Complex, Aromatic-Rich Fractions from Biomass. ChemSusChem 13, 1-14 (2020).
43. Bator, I., Wittgens, A., Rosenau, F., Tiso, T. & Blank, L. M. Comparison of three xylose pathways in Pseudomonas putida KT2440 for the synthesis of valuable products. Front. Bioeng. Biotechnol. 7, 480 (2019).
44. Lim, H. G. et al. Generation of Pseudomonas putida KT2440 Strains with Efficient Utilization of Xylose and Galactose via Adaptive Laboratory Evolution. ACS Sustain. Chem. Eng. 9, 11512-11523 (2021).
45. Elmore, J. R. et al. Engineered Pseudomonas putida simultaneously catabolizes five major components of corn stover lignocellulose: Glucose, xylose, arabinose, p-coumaric acid, and acetic acid. Metab. Eng. 62, 62-71 (2020).
46. Klamt, S., Saez-Rodriguez, J. & Gilles, E. D. Structural and functional analysis of cellular networks with CellNetAnalyzer. BMC Syst. Biol. 1, 2 (2007).
47. Ebrahim, A., Lerman, J. A., Palsson, B. O. & Hyduke, D. R. COBRApy: COnstraints-Based Reconstruction and Analysis for Python. BMC Syst. Biol. 7, 74 (2013).
48. Kukurugya, M. A. et al. Multi-omics analysis unravels a segregated metabolic flux network that tunes co-utilization of sugar and aromatic carbons in Pseudomonas putida. J. Biol. Chem. 294, 8464-8479 (2019).
49. Heirendt, L. et al. Creation and analysis of biochemical constraint-based models using the COBRA Toolbox v.3.0. Nat. Protoc. 14, 639-702 (2019).
50. Marx, C. J. Development of a broad-host-range sacB-based vector for unmarked allelic exchange. BMC Res. Notes1, 1 (2008).
51. Chen, Y. et al. Automated “Cells-To-Peptides” Sample Preparation Workflow for High-Throughput, Quantitative Proteomic Assays of Microbes. J. Proteome Res. 18, 3752-3761 (2019).
52. Chen, Y. et al. A rapid methods development workflow for high-throughput quantitative proteomic applications. PLOS ONE14, e0211582 (2019).
53. Sharma, V. et al. Panorama: a targeted proteomics knowledge base. J. Proteome Res. 13, 4205-4210 (2014).
54. Korz, D. J., Rinas, U., Hellmuth, K., Sanders, E. A. & Deckwer, W. D. Simple fed-batch technique for high cell density cultivation of Escherichia coli. J. Biotechnol. 39, 59-65 (1995).
55. Magurudeniya, H. D. et al. Use of ensiled biomass sorghum increases ionic liquid pretreatment efficiency and reduces biofuel production cost and carbon footprint. Green Chem. 23, 3127-3140 (2021).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified Pseudomonas cell is capable of producing isoprenol comprising (a) one or more, or all, of heterologous genes encoding: MvaE, AtoB, MvaS, MK, PMD_{HKQ}, AphA, and PhoA; and (b) a deletion, knock out, or reduced in expression of one or more of the following endogenous genes: a gene at PP_2675 locus, or a deletion of the PP_2675 locus, phaABC, mvaB, hbdH, IdhA, gntZ, ppsA, pycAB, gltA, and aceA.

2. The genetically modified Pseudomonas cell of claim 1, further capable of producing epi-isozizaene (C₁₅).

3. A medium comprising (a) the genetically modified Pseudomonas cell of claim 1, and (b) one or more amino acids that reduce the catabolism of isoprenol.

4. The medium of claim 3, wherein the one or more amino acids that reduce the catabolism of isoprenol is glutamate, glutamine, arginine, glycine, serine, valine leucine, and/or alanine, or a mixture thereof.

5. The medium of claim 4, wherein the one or more amino acids that reduce the catabolism of isoprenol is glutamate and/or glutamine.

6. A method to increase production of isoprenol by a genetically modified Pseudomonas cell, the method comprising: (a) providing a genetically modified Pseudomonas cell comprising one or more, or all, of heterologous genes encoding: MvaE, AtoB, MvaS, MK, PMD_{HKQ}, AphA, and PhoA; and (b) culturing or growing the genetically modified Pseudomonas cell in a medium to produce isoprenol; wherein (i) the genetically modified Pseudomonas cell is deleted, knocked out, or reduced in expression of one or more of the following endogenous genes: a gene at PP_2675 locus (or a deletion of the PP_2675 locus), phaABC, mvaB, hbdH, IdhA, gntZ, ppsA, pycAB, gltA, and aceA, and/or (ii) the medium comprises one or more amino acids that reduce the catabolism of isoprenol.

7. The method of claim 6, wherein the genetically modified Pseudomonas cell is capable of producing epi-isozizaene (C₁₅), and the culturing or growing step (b) comprises the genetically modified Pseudomonas cell produces epi-isozizaene (C₁₅).
